(19) United States
(12) Patent Application Publication
(10) Pub. No.: US 2026/0234688 A1
(43) Pub. Date: Aug. 13, 2026

(54) NISIN-PERMEABILIZED MICROBIAL CELL CATALYSTS

(71) Applicant: DANMARKS TEKNISKE UNIVERSITET, Kongens Lyngby (DK)

(72) Inventors: Peter Ruhdal Jensen, Kongens Lyngby (DK); Christian Solem, Kongens Lyngby (DK); Jianming Liu, Kongens Lyngby (DK); Robin Dorau, Kongens Lyngby (DK); Qi Wang, Kongens Lyngby (DK); Ge Zhao, Kongens Lyngby (DK)

(21) Appl. No.: 19/530,826

(22) Filed: Feb. 5, 2026

Related U.S. Application Data
(62) Division of application No. 17/795,983, filed on Feb. 12, 2021, now Pat. No. 12,571,019.

(30) Foreign Application Priority Data
Feb. 13, 2020 (EP) 20157131.2

(51) Int. Cl. C12P 21/02; A23C 9/12; C12N 9/38

(52) U.S. Cl. CPC C12P 21/02; A23C 9/1206; C12N 9/2471; C12Y 302/01023; A23V 2400/231; A23V 2400/249

(57) ABSTRACT

A method and use of nisin-permeabilized microbial cells as whole-cell catalysts for reducing the amount of a target substrate in a sample to one of more product are provided. Specifically, a method of reducing the amount of lactose in a dairy sample using nisin-permeabilized lactic acid bacterial cell catalysts, which have been permeabilized by incubating with a nisin producing microbial cell and/or culture medium derived thereof. Further provided is a nisin producing microbial cell, derived from parent strain Lactococcus lactis subsp. lactis bv. diacetylactis SD96 (NCBI accession No. SRX6686433).

### CROSS REFERENCE TO RELATED APPLICATIONS

This application is a divisional application of U.S. patent application Ser. No. 17/795,983, filed Jul. 28, 2022, now allowed, which is a national stage application of PCT/EP2021/053505, filed Feb. 12, 2021, which claims priority to European Application No. 20157131.2, filed Feb. 13, 2020, each of which are hereby incorporated by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 178,071 Byte XML file named “GUARD_41149_402_SequenceListing.xml,” created on Feb. 5, 2026.

### FIELD OF THE INVENTION

The present invention relates to use of nisin-permeabilized microbial cells as whole-cell catalysts for conversion (partial or full) of a target substrate(s) to one or more products. More specifically, the invention relates to a method for reducing the amount of lactose in a dairy sample, wherein a microbial cell catalyst comprising beta-galactosidase is first prepared by nisin-permeabilization by incubating with a nisin producing microbial cell or a culture medium thereof, prior to incubating with said dairy sample for hydrolyzing said lactose. The invention further relates to a nisin producing microbial cell for producing a microbial cell catalyst.

### BACKGROUND OF THE INVENTION

Many microbial enzymes with a potential for industrial application are retained within the microbial cells in which they are produced, which makes recovery and subsequent purification difficult.

Further, enzymes, while inside cells, are in a protected environment and are often more stable than when isolated. But for use of such enzyme(s) inside the cells, the substrate(s) must be able to cross the cell envelope to reach the enzyme(s), which may decrease the reaction rate obtained with such cells when compared to the corresponding isolated enzyme. One way to circumvent substrate transfer limitations involves the permeabilization of the cell wall and membranes by a chemical (e.g. by adding detergents or solvents) or physical (e.g. temperature shock) treatment. However, these procedures may interfere with the manufacturing and downstream processes, besides damaging the cells.

These problems are illustrated by the enzyme beta-galactosidase of Streptococcus thermophilus which, because of its food-grade classification, has attractive possibilities for application in the production of low-lactose foods. Since beta-galactosidase in S. thermophilus is a cytoplasmic enzyme, its isolation and use as a catalyst may only be achieved through the perturbation of cell integrity, either by sonic disruption or by chemical treatment such as using solvent mixtures. Sonication of the cells results in the release of beta-galactosidase, whereas permeabilization with organic solvents allows the passage of lactose to the cell interior while the beta-galactosidase is retained within the cell. Permeabilized cells may be used in place of purified beta-galactosidase in the production of low-lactose foods. Somkuti et al., 1996 found that several detergents and the commercial bile salt preparation Oxgall were effective in disrupting membrane structures in S. thermophilus to allow lactose influx but without causing enzyme leakage or denaturation. However, naturally a concern must be addressed in relation to the use of such chemicals in relation to food products, as residual permeabilizing agents may remain associated with the concentrated cell preparations intended for food applications (even after washing the cells).

The main concern when using chemical permeabilization of microbial cultures in the preparation of foods or beverages is solvent or detergent residues remaining associated with treated cells and ending up in finished food or beverage products. E.g. organic solvents such as toluene or acetone-toluene mixtures are excellent permeabilizing agents, but their residues would surely find objection when used in foods. In this regard, ethanol may be a better solvent choice, since it is already present in trace amounts in many fermented dairy foods consumed by humans. Somkuti et al., 1998 found that ethanol is efficient as a permeabilizing agent for increasing the level of measurable beta-galactosidase activity.

However, traces of any solvent, even ethanol, in a food or beverage, due to their treatment with permeabilized cells during manufacture, is considered undesirable and hence there remains a need to provide food grade microbial cell catalysts that avoid these problems.

### SUMMARY OF THE INVENTION

In a first aspect, the present invention provides a method for reducing the amount of lactose in a first dairy sample, said method comprising the steps of:
a) providing one or more non-GMO microbial cells comprising an intracellular beta-galactosidase enzyme for catalyzing conversion of lactose into glucose and galactose,
b) incubating said non-GMO microbial cells with a nisin producing microbial cell culture and/or a culture medium derived thereof,
c) optionally harvesting permeabilized non-GMO microbial cell catalysts obtained in step (b),
d) incubating permeabilized non-GMO microbial cell catalysts obtained in step (b) or harvested non-GMO microbial cell catalysts harvested in step (c) with said first dairy sample comprising lactose;
wherein said non-GMO microbial cell is a lactic acid bacterium

In a second aspect, the invention provides a nisin producing microbial cell derived from parent strain Lactococcus lactis subsp. lactis bv. diacetylactis SD96 (NCBI accession No. SRX6686433) by virtue of inserting transposon Tn5307 (SEQ ID NO. 4) comprising a nisin biosynthesis gene cluster and genes needed for metabolizing sucrose into the genome of the parent strain.

In a third aspect, the invention provides nisin producing microbial cell Ge001 for producing a microbial cell catalyst.

In a fourth aspect, the invention provides the use of a nisin producing microbial cell of the invention for producing a microbial cell catalyst, preferably a non-GMO microbial cell comprising an intracellular beta-galactosidase enzyme.

### DESCRIPTION OF THE INVENTION

### Brief Description of the Figures

FIG. 1: Illustration of lactose hydrolysis by nisin-permeabilized cell.

FIGS. 2A and 2B: Lactose hydrolysis by S. thermophilus CS1980. Cells (100 mg/l) were permeabilized at 28° C. (FIG. 2A) or 37° C. (FIG. 2B) for 10 min using a nisin concentration of 2.5 μg/ml. Hydrolysis was subsequently performed using 100 mg/l dry weight of permeabilized cells in a POM buffer solution comprising 5% lactose, incubated at temperatures ranging 20-60° C.

FIG. 3: Lactose hydrolysis by different S. thermophilus strains. Cells (100 mg/l) were permeabilized at 28° C. for 10 min using a nisin concentration of 2.5 μg/ml. Hydrolysis was subsequently performed at 50° C., using 100 mg/l dry weight of permeabilized cells in POM buffer solution comprising 5% lactose.

FIG. 4: Hydrolysis of lactose in milk by S. thermophilus ST057-1 cells using different cell concentrations: either 100 mg/l or 1 g/l. Permeabilization was performed at 37° C. for 10 min using a nisin concentration of 2.5 μg/ml. Hydrolysis of milk-lactose was subsequently performed at 50° C.

FIGS. 5A and 5B: Hydrolysis of lactose by S. thermophilus CS1980 using different permeabilization temperatures: 28, 50, and 55° C. Nisin-permeabilization was performed using 2.5 μg/ml nisin in POM buffer for 10 minutes. Hydrolysis of lactose in POM was performed at 50° C., using nisin-treated cells (FIG. 5A) or non-nisin-treated cells (FIG. 5B) at a cell concentration of approximately 100 mg/l (dry cell weight).

FIG. 6: Hydrolysis of lactose in milk by S. thermophilus ST057-1 using different permeabilization temperature: 28° C. or 37° C. Nisin-permeabilization was performed using 2.5 μg/ml nisin in POM buffer for 10 minutes at 28° C. or 37° C. Hydrolysis of lactose in milk was performed at 50° C., using nisin-treated cells at a cell concentration of approximately 1 g/l.

FIG. 7: Hydrolysis of lactose by S. thermophilus CS1980 where the cells have been permeabilized for 10 minutes at 28° C. using (i) 2.5 μg/ml nisin in POM buffer or (ii) 45% (v/v) aqueous ethanol, or (iii) have not been permeabilized (control). Hydrolysis of lactose in POM buffer (50 g/I lactose) at 50° C. was carried out at a cell concentration of approximately 100 mg/l.

FIG. 8: Alpha-acetolactate synthase activity of nisin-permeabilized L. lactis strain MG1363 ΔldhB ΔldhX Δpta ΔadhE ΔbutBA ΔaldB, harboring an expression vector for overexpressing native alpha-acetolactate synthase; compared to activity of non-permeabilized cells.

FIG. 9: Hydrolysis of lactose by S. thermophilus CS1980 where the cells have been permeabilized in POM buffer pH 7.4 for 10 minutes at 28° C. using (i) 0.25 μg/ml nisin, (ii) 0.25 μg/ml nisin+10 μg/ml monolaurin, or (iii) not permeabilized (control—no addition of nisin or monolaurin).

FIGS. 10A and 10B: Performance and nisin production kinetics for Ge001 cultivated in 10% ML 1% YE. (A) Optical density at 600 nm (OD600); (B) pH change; (C) nisin production; (D) lactate and acetoin production. All fermentations were carried out in 25 mL medium using 300 mL shake flasks, two times independently. Error bars indicate standard deviations.

FIG. 11: Effect of Ca2+ concentration (0-300 mM) on the performance of Ge001 using 20% ML, 2% YE containing 1 μg/mL hemin and 1 mM Mn2+. The initial OD600 was 0.05. All fermentations were carried out in 25 mL medium using 300 mL shake flasks and samples were extracted at 12 hours cultivation. (A) Optical density at 600 nm (OD600). (B) Nisin titers. The values are averages of two independent experiments and error bars indicate standard deviations.

FIG. 12: Nisin production and lactose consumption by Ge001 grown in UHT milk. The fermentations were carried out in 25 mL UHT milk (1.5% fat content) using 300 mL shake flasks. The values are averages of two independent experiments.

FIG. 13: Hydrolysis of lactose in UHT milk (1.5% fat content) by permeabilized Streptococcus thermophilus cells, wherein the cells have been permeabilized by incubating for 30 min at 30° C. with (1) POM buffer with 2.5 μg/ml nisin (commercial), (2) milk cultured with Ge001 (100%), (3) two times diluted milk cultured with Ge001 (50%), (4) ten times diluted milk cultured with Ge001 (10%). All dilutions of nisin culture were done using POM buffer. Harvested permeabilized cells were resuspended in 1 ml fresh UHT milk (1.5% fat), and incubated at 50° C. Samples were withdrawn regularly (0 h, 0.5 h, 1 h, and 2 h) and analyzed using HPLC. The experiment was carried out using three independent replicates.

### Abbreviations, Terms, and Definitions

Nisin-permeabilized cells are cells which have been treated with nisin whereby pores have formed in the membrane through which a target substrate can transit while enzymes that catalyze the conversion of the substrate are retained within the cells.

Susceptible to nisin-permeabilization defines a property of a cell that on interaction with nisin, allows nisin to form a pore in its cell membrane through which target substrates can transit while enzymes which catalyze the conversion of the target substrates are retained within the cell; such cell being defined as susceptible to nisin-permeabilization.

Class A lantibiotics are bacteriocins that disrupt bacterial cell walls by pore formation. Class A lantibiotics include nisin, bisin, subtilin, epidermin, gallidermin, and mutacin.

### DETAILED DESCRIPTION OF THE INVENTION

### I. Nisin-Permeabilized Microbial Cell Catalysts

The microbial cell according to the invention is a cell which is susceptible to nisin-permeabilization—in other words, a cell with which nisin interacts to form a pore in the cell's cell membrane.

The microbial cell of the invention is preferably a bacterium, such as a Gram-positive or Gram-negative bacterium. A non-exhaustive list of suitable bacteria is given as follows: a species belonging to the genus selected from among Escherichia, Streptococcus, Lactobacillus, Lactococcus, Lactovum, Pediococcus, Leuconostoc, Fructobacillus, Weissella, Oenococcus, Corynebacterium, Brevibacterium, Bacillus, Sporolactobacillus, Geobacillus, Halobacillus, Halolactibacillus, Tetragenococcus, Acetobacter, Acinetobacter, Proprionibacterium, and Bifidobacterium.

In one embodiment, the microbial cell of the invention is selected from lactic acid bacteria, such as from the group consisting of species of the genera Streptococcus, Lactobacillus, Lactococcus Abiotrophia, Aerococcus, Carnobacterium, Enterococcus, Leuconostoc, Oenococcus, Pediococcus, Tetragenococcus, Vagococcus, and Weissella. Even more preferably, in one embodiment, the microbial cell of the invention is selected from Streptococcus thermophilus, Lactobacillus casei, Lactobacillus plantarum, Lactobacillus helveticus, Lactobacillus delbrueckii, Lactobacillus acidophilus, and Lactococcus lactis.

While not wishing to be bound to theory, the action mechanism of nisin consists in the adsorption of nisin on the target cell surface and destabilization of the cytoplasmic membrane structure through the insertion of nisin in the lipid cell membrane leading to its permeabilization. The cells become “leaky” as a result of the nisin treatment, and release of e.g. essential cytoplasm components, and/or cell lysis, eventually results in the bacterium death.

The present invention is based on the novel idea of exploiting the ability of nisin to puncture holes (pores off approx. 2-2.5 nm in diameter) in the cytoplasmic cell membrane of microbial cells, in order to create non-growing cells (non-viable) that can find a new use as whole cell catalysts.

The nisin-permeabilized microbial cell is used as a permeabilized whole-cell catalyst, where substrate can enter the nisin-permeabilized cells though the nisin pores and undergo enzymatic catalysis by a suitable enzyme within the cell, and products of the enzymatic reaction can then optionally also exit the cell through the nisin pores. Small molecules thereby transit though the nisin pores, while larger molecules (such as intracellular enzymes) are retained. The enzyme is thus kept within a stabilizing cell bag, which can easily be recovered from a solution by simple filtration.

### II. Methods for Preparing Nisin-Permeabilized Microbial Whole-Cell Catalysts

In preparing nisin-permeabilized cells, microbial cells of the invention are simply brought in contact with nisin, such as nisin being added to a suspension of the microbial cells. Nisin will insert into the lipid cell membrane and lead to its permeabilization by formation of a pore in the membrane.

### II.i Providing Nisin for Permeabilizing Cells

In one embodiment of the invention, nisin for permeabilizing cells of the invention is provided in purified form, such as commercially available nisin: e.g. Nisaplin® from Dupont Nutrition & Biosciences, Delvo®Nis from DSM, Niseen® from Siveele B. V., Galacin® from Galactic, Nisin from Shandong Freda Biotechnology Co., Ltd., Nisin from Zhejiang Silver-Elephant Bio-Engineering Co., Ltd., and NisinPro from Chihon Biotechnology Co., Ltd.

In another embodiment, nisin is provided in the form of a nisin producing microbial strain or a culture medium derived from a nisin producing strain. The strain may be genetically modified to produce nisin or be a natural producer of nisin. Nisin is for example naturally produced by certain Lactococcus species, which may be used in the present invention for providing nisin. In one embodiment, nisin-producing Lactococcus species or culture media derived therefrom are used as a source of nisin. In a preferred embodiment, a nisin-producing Lactococcus lactis strain or culture media derived therefrom is used as a source of nisin.

In a preferred embodiment of the invention, the nisin producing microbial strain is a strain modified to produce nisin by classical mutagenic methods, hence a non-GMO strain. Such classical mutagenic methods may involve adaptive laboratory evolution, chemical mutagenesis and/or conjugation. Such strains are considered “natural” and thus can be applied in e.g. dairy fermentation without any restrictions. However, the strain can also be obtained by genetic engineering using gene recombination methods to introduce the desired genetic modifications using known in the art, including CRISPR.

In one embodiment, the nisin producing strain comprises a nisin gene encoding a nisin polypeptide having at least 80, 82, 84, 86, 88, 90, 92, 94, 96, or 98% amino acid sequence identity to SEQ ID No. 2.

A nisin gene cluster encoding nisin as well as genes for sucrose metabolism are located on a conjugative transposon in L. lactis (Broadbent et al 1995); this transposon can ‘jump’ from one strain and ‘insert’ itself into another strain by conjugal transfer. In one embodiment, the nisin producing microbial strain is prepared by transposon conjugation. In a preferred embodiment, the nisin producing microbial strain is obtained by transferring the nisin gene cluster of L. lactis ATCC 11454 (SEQ ID No. 3), by conjugation, into a lactate dehydrogenase (LDH) deficient strain as specified above.

In a preferred embodiment, the nisin producing microbial strain comprises a nucleic acid sequence having at least 80, 82, 84, 86, 88, 90, 92, 94, 96, or 98% nucleic acid sequence identity to transposon Tn5307 (SEQ ID No. 4).

In one embodiment, the nisin producing strain is additionally characterized in having reduced lactate dehydrogenase (LDH) activity when compared to the parent strain from which it was derived. Reduced LDH activity can block the main metabolic flux from pyruvate to lactate and thus reduce acid production. Such strain having reduced lactate dehydrogenase activity can be grown to high cell densities without pH control.

In a preferred embodiment, the Idh gene encoding lactate dehydrogenase enzyme having at least 80, 82, 84, 86, 88, 90, 92, 94, 96, or 98% amino acid sequence identity with SEQ ID NO. 6 is disrupted in the nisin producing strain, such as by one or more mutations in the Idh gene, by deletion of the Idh gene or parts thereof, by insertion of one or more nucleic acids into the Idh gene leading to a translated peptide which does not have LDH activity, or by other means of gene disruption as recognized by a person skilled in the art.

In one embodiment, the Idh gene is disrupted by insertion of one or more nucleotides in the Idh gene. In a specific embodiment, the nisin producing strain is a lactic acid bacterium comprising the nucleotides CCGTCAAG inserted between nucleotide T464 and C465 in the CDS region of the parent Idh gene (SEQ ID No. 5), hence resulting in a frameshift change.

In a one embodiment, the nisin producing strain is a strain derivable from the parent strain lactic acid bacterium Lactococcus lactis subsp. lactis bv. diacetylactis SD96 (NCBI accession No SRX6686433), characterized by the ability to produces nisin and being devoid of lactate dehydrogenase activity, by virtue of the following genetic modification in the genome when compared to the genome of the parent strain:
I. a transposon Tn5307 (SEQ ID NO. 4) comprising nisin biosynthesis gene cluster and genes needed for metabolizing sucrose is inserted into the parent genome,
II. nucleic acid sequence CCGTCAAG is inserted between nucleotide T464 and C465 in the CDS region of the parent Idh gene (SEQ ID No. 5) encoding lactate dehydrogenase,

In a further embodiment, the nisin producing microbial strain is capable of growing in milk and/or stream derived from milk processing. Milk may be any kind of milk, such as skimmed milk, regular milk, whole milk, including ultrahigh temperature (UHT) treated milk and pasteurized milk. Milk derived streams may be any stream derived from milk production, such as a waste stream, for example a stream comprising whey, e.g. whey mother liquor. In a preferred embodiment, the nisin producing strain is a lactic acid bacterium adapted to grow in milk and/or a stream derived from milk.

The dairy industry generates significant volumes of low-value side-streams, i.e. dairy waste. One of these is whey mother liquor (ML), which is a remaining product of whey processing and which mainly contains lactose along with citrate and different salts. Worldwide whey production is estimated to be around 1.8 to 1.9×10⁸ ton/year, and quite large amounts of ML are available worldwide. These whey side-streams are often challenging to dispose of, due to their high organic load, and the Chemical Oxygen Demand (COD) can be as high as 100,000 mg O₂ L⁻¹. There is a great potential in transforming these waste materials into high value-added products.

In one embodiment, the nisin producing microbial strain is capable of growing in milk and/or a stream derived from milk at elevated temperatures, such as temperatures at least up to 40° C. In one embodiment, the nisin producing microbial strain is capable of growing at temperatures up to 30, 31, 32, 33, 34, 35, 36, 37, 38, 39, or even 40° C.

In one embodiment, the nisin producing strain is a lactic acid bacterium that is derivable from Lactococcus lactis subsp. lactis bv. diacetylactis SD96, and is characterized by the ability to produce nisin as well as the ability to grow in milk, preferably at elevated temperatures. The acquisition of these characteristics may be achieved by targeted mutation (CRISPR editing) or for example by adaptive evolution.

Accordingly, in one embodiment, the nisin producing strain is a strain derivable from the parent strain lactic acid bacterium Lactococcus lactis subsp. lactis bv. diacetylactis SD96 (NCBI accession No SRX6686433), that is characterized by the ability to produce nisin and grow in milk at elevated temperatures (such as at least up to 40° C.), by virtue of the following genetic modifications in the genome when compared to the genome of the parent strain:
I. a transposon Tn5307 (SEQ ID NO. 4) comprising the nisin biosynthesis gene cluster and genes needed for metabolizing sucrose is inserted into the parent genome,
II. a parent gene encoding UDP-N-acetylmuramate-L-alanine ligase of SEQ ID No. 8 is modified to encode said amino acid sequence having substitution F68L,
III. a parent gene encoding GTP pyrophosphokinase (ReIA) of SEQ ID No. 10 is modified to encode said amino acid having substitution V469L,
IV. base pairs 1,823,878-1,897,135 (SEQ ID No. 11) in the parent genome are deleted, and
V. a tandem repeat ((A)6 to (A)5) upstream of the CodY transcription regulator (SEQ ID No. 12) in the parent genome is deleted.

In a preferred embodiment, the nisin producing strain is a strain derivable from the parent strain lactic acid bacterium Lactococcus lactis subsp. lactis bv. diacetylactis SD96 (NCBI accession No SRX6686433), characterized by:
(i) the ability to produces nisin by virtue of the following genetic modification in the genome when compared to the genome of the parent strain:
   I. a transposon Tn5307 (SEQ ID NO. 4) comprising nisin biosynthesis gene cluster and genes needed for metabolizing sucrose is inserted into the patent genome,
(ii) the ability to grow in milk at elevated temperatures (such as at least up to 40° C.), by virtue of the following genetic modifications in the genome when compared to the genome of the parent strain:
   II. a parent gene encoding UDP-N-acetylmuramate-L-alanine ligase of SEQ ID No. 8 is modified to encode said amino acid sequence having substitution F68L,
   III. a parent gene encoding GTP pyrophosphokinase (ReIA) of SEQ ID No. 10 is modified to encode said amino acid having substitution V469L,
   IV. base pairs 1,823,878-1,897,135 (SEQ ID No. 11) in the parent genome are deleted, and
   V. a tandem repeat ((A)6 to (A)5) upstream of the CodY transcription regulator (SEQ ID No. 12) in the parent genome is deleted,
and (iii) being devoid of lactate dehydrogenase activity by virtue of the following genetic modification in the genome when compared to the genome of the parent strain:
   VI. nucleic acid sequence CCGTCAAG is inserted into the CDS region of the parent Idh gene (SEQ ID No. 5) encoding lactate dehydrogenase between nucleotides T464 and C465,

As mentioned above, the ability of the nisin producing strain to produce nisin is preferably obtained by classical mutagenic methods, hence a non-GMO strain.

In a much preferred embodiment, the nisin-producing microbial strain is Ge001, deposited with depository institution DSMZ German collection of microorganism and cell cultures, Inhoffenstraße 7B, 38124 Braunschweig, GERMANY, under the Budapest Treaty having Deposit Number XXX. Ge001 produces nisin and is deficient in lactate dehydrogenase activity, and can therefore be grown to high cell densities without pH control. Ge001 is further especially preferred due to its ability to grow in milk and milk derived streams, such as illustrated in examples 8-10.

### II.ii Permeabilization Conditions

Permeabilization may be performed within a wide temperature range—such as demonstrated in Example 4. It is preferred to avoid temperatures that are sufficiently high that they compromise cell and enzyme stability, rather than using a temperature that is optimal for nisin permeabilization as such. Permeabilization temperature for mesophilic organisms is therefore preferably lower than 60° C., such as lower than 55° C., preferably even lower than 54, 53, 52, 51, of 50° C. Meanwhile for thermophilic organisms the permeabilization temperatures may be relatively higher, depending on the specific microorganism.

Without being bound by theory, the nisin permeabilization reaction happens as soon as the cells are brought in contact with the nisin. In other words, in a well-mixed nisin saturated solution, the cells will quickly become permeabilized, and extended permeabilization reaction time is not needed. This is, for example, demonstrated in Example 4, where 30 minutes permeabilization time showed no significant improvement compared to 10 minutes. In one embodiment, the permeabilization time may be 5, 10, 15, 20, 25, 30, 35, 40, 45, 50, 55, or 60 seconds, In one embodiment, permeabilization time may be 1-60 minutes, such as 1-45 minutes, 1-30 minutes, 1-20 minutes; or even 1-10 minutes permeabilization time may suffice.

The amount/concentration of nisin applied for sufficient permeabilization of cells may depend on permeabilization conditions, such as the type of microbial cell to be permeabilized, concentration of cells, permeabilization time, and other medium conditions.

As an illustrative example, when 100 mg/l S. thermophilus cells are to be permeabilized: the examples provided herein show that 2.5 μg/ml nisin in POM buffer efficiently permeabilizes the cells within merely 10 minutes. Increasing the cell concentration may require increased nisin concentration to obtain efficient permeabilization within the same time period. Meanwhile, if the cell concentration is increased, but nisin concentration is maintained, then the permeabilization time can be increased to obtain sufficient permeabilization. A person skilled in the art would know how to adjust/optimize these different parameters to obtain optimal permeabilization for a given organism at a desired cell concentration.

In one embodiment, a nisin concentration of 2-250 μg nisin per mg cells is used in permeabilization of the cells, such as a nisin concentration of 10-150 μg nisin per mg cells, such as a nisin concentration of 10-100 μg nisin per mg cells, such as a nisin concentration of 20-50 μg nisin per mg cells, such as preferably around 25 μg nisin per mg cells.

In a preferred embodiment, 0.1 g/l cells are treated with around 2.5 μg/ml nisin for around 10 minutes.

After nisin-treatment, the cells may be used directly as permeabilized whole-cell catalysts, or they may preferably first be purified prior to use. A person skilled in the art would know how to perform such cell purification. Purification may simply be performed by pelleting the cells by centrifugation and removing the top liquid portion; or such as by simple filtration. The cells may further be washed, if needed, prior to their intended application. Any excess nisin left in the permeabilization medium may after harvesting the permeabilized cells be used in the subsequent permeabilization treatments of other cells.

In one embodiment, the nisin-permeabilized cells may be prepared as described above and further treated by a method ensuring preservation and possible storage of the cells prior to use. Methods of preservation, such as drying, freezing, or preparing liquid stocks of the microbial cells are known by a person skilled in the art and include, for example, glycerol stocks, or freezing concentrated cell slurries in liquid nitrogen, freeze-drying, spray drying, vacuum drying, etc.

### II.iii Nisin-Permeabilized Microbial Whole-Cell Catalysts

One aspect of the present invention relates to a whole-cell catalyst comprising nisin-permeabilized microbial cells; wherein the nisin-permeabilized cells comprise at least one intracellular enzyme for catalyzing conversion of a target substrate; and wherein the nisin-permeabilized cells are in a frozen or dried state.

Such whole-cell catalysts may be provided in bags, ampoules, tubes, vial, or the like depending on consumer preference.

The whole-cell catalyst may comprise any microbial cell susceptible to nisin-permeabilization comprising an enzyme capable of catalyzing the conversion of a substrate, provided that (i) the size/shape/conformation of the enzyme ensures it being retained within the cell (not transiting though the nisin pores), and (ii) the size/shape/conformation of the substrate allows for the substrate to transit through nisin pores of the cell membrane.

In one embodiment, the whole-cell catalyst comprises frozen or dried preparations of nisin-permeabilized lactic acid bacteria comprising beta-galactosidase EC 3.2.1.23 for catalyzing conversion of lactose to glucose and galactose.

In one embodiment, the whole-cell catalyst comprises frozen or dried preparations of nisin-permeabilized bacteria comprising arabinose isomerase EC. 5.3.1.4 for isomerization of galactose to tagatose.

In one embodiment, the whole-cell catalyst comprises frozen or dried preparations of nisin-permeabilized bacteria comprising xylose (glucose) isomerase EC 5.3.1.5 for isomerization of glucose to fructose.

In one embodiment, the whole-cell catalyst comprises frozen or dried preparations of nisin-permeabilized bacteria comprising alpha-acetolactate decarboxylase EC 4.1.1.5 for conversion of alpha-acetolactate into acetoin.

In one embodiment, the whole-cell catalyst comprises frozen or dried preparations of nisin-permeabilized bacteria comprising alpha-acetolactate synthase EC 2.2.1.6 for conversion of pyruvate into alpha-acetolactate.

### III. Method for Reducing the Content of a Substrate in a Sample Using Permeabilized Microbial Cell Catalysts

One aspect of the present invention relates to a method for reducing the content of a substrate in a sample, said method comprising the steps of:
a. providing microbial cells comprising an intracellular enzyme for catalyzing conversion of said substrate into one or more products,
b. incubating said microbial cells with nisin,
c. optionally harvesting permeabilized cells obtained in step (b),
d. incubating permeabilized cells obtained in step (b) or harvested cells obtained in step (c) with said sample comprising said substrate;
   wherein said microbial cells are susceptible to nisin-permeabilization, and wherein the substrate can transit through nisin pores of the permeabilized cells.

Nisin-permeabilized microbial cells are hereby used as a whole-cell catalyst. Whereas prior art has only used nisin as a means for killing cells—such as to avoid food spoilage by contaminating microbes, the present invention surprisingly reveals that nisin-permeabilized cells are excellent whole-cell catalysts as the cells function as a porous “bag” retaining and protecting the enzymes while substrates can freely transit across the cell membrane via the nisin pores to be hydrolyzed within the cell. The products of the enzymes reaction may transit out through the nisin pores into the medium or be consumed by the cell such as used by the cells metabolic machinery.

Depending on the application, it may be desirable to harvest the nisin-permeabilized cells prior to use to either be able to re-use excess nisin or to simply avoid having nisin carry-over in the catalytic reaction by the nisin-treated cell catalysts. In other embodiments, the presence of nisin in the catalytic reaction by the nisin-treated cell catalysts is of no concern, and nisin treatement (step b) and incubation with the substrate (step d) may therefore occur simultaneously, rather than as separate steps.

Numerous different applications exist for the method of the present invention. Any microbial cell susceptible to nisin-permeabilization may be used in the present method for conversion (reducing the amount) of a target substrate in a sample, provided that the cell comprises an enzyme capable of catalyzing the conversion of the substrate and that the size/shape/conformation of the substrate allows for the substrate to transit through nisin pores in the cell membrane, while the size/shape/conformation of the enzyme ensures it being retained within the cell (not transiting though the nisin pores). The nisin pores are expected to have an approximate size of 2-2.5 nm.

In one embodiment, suitable applications of the present invention involves an enzyme catalyst having its smallest dimension being at least 1, 1.5, 2, 2.5, 3, 3.5, 4, 4.5, or 5 nm, for the enzyme to be retained within the cell, while the largest dimension of the substrate of the catalyst correspondingly does not exceed 1, 1.5, 2, 2.5, 3, 3.5, 4, 4.5, or 5 nm, for the substrate to be able to transit through the nisin pores into the cell.

Embodiments of such applications of the invention may comprise hydrolysis of lactose by bacteria comprising beta-galactosidase; isomerization of galactose to tagatose using a bacterium comprising arabinose isomerase; isomerization of glucose to fructose using a bacterium comprising xylose (glucose) isomerase; conversion of alpha-acetolactate into acetoin using a bacterium comprising alpha-acetolactate decarboxylase; conversion of pyruvate into alpha-acetolactate using a bacterium comprising alpha-acetolactate synthase; etc.

The above applications are merely illustrative—as mentioned previously, the method has very broad application since the use of whole-cell catalysts of the invention for catalysis avoids the problems associated with enzyme secretion or purification. Furthermore, the enzymes in the whole cell catalysts of the invention are better protected and thus more active than secreted or isolated intracellular enzymes. The microorganisms applied in the method may naturally comprise the needed enzyme(s) for conversion of a target substrate, or they may be genetically modified by standard methods known in the art to express the required enzyme(s).

In a preferred embodiment, the application of the method of the present invention is within food industries, such as where the substrate of the invention is a food or beverage. Natural, non-GMO microbes are preferred for such application.

In one embodiment, the present method is suitable for reducing the lactose content of a dairy product—such as for producing lactose-reduced milk, yoghurt (and yoghurt like products, e.g. Gaio®, Cultura®), Skyr, Quark, Greek yoghurt, butter milk, cream, butter, whey, and other dairy products.

Specifically, such method of the present invention for reducing the lactose content of a dairy product comprises the steps of:
a. providing lactic acid bacteria comprising beta-galactosidase EC 3.2.1.23. for catalyzing conversion of lactose to galactose and glucose,
b. incubating said bacteria with nisin,
c. optionally harvesting permeabilized bacteria obtained in step (b),
d. incubating permeabilized bacteria obtained in step (b) or (c) with said dairy product.

In a preferred embodiment, the method of the present invention for reducing the amount of lactose in a dairy sample comprising the steps of:
a) providing one or more non-GMO microbial cells comprising an intracellular beta-galactosidase enzyme for catalyzing conversion of lactose into glucose and galactose,
b) incubating said non-GMO microbial cell with a nisin producing second microbial cell culture and/or a culture medium derived thereof,
c) optionally harvesting permeabilized non-GMO microbial cell catalysts obtained in step (b),
d) incubating permeabilized non-GMO microbial cell catalysts obtained in step (b) or harvested non-GMO microbial cell catalysts harvested in step (c) with said first dairy sample comprising lactose;
wherein said first microbial cell is a lactic acid bacterium

In one embodiment, the nisin producing microbial cell culture is obtained by culturing a nisin producing microbial cell in a second dairy sample, prior to incubating with the second microbial cell in step (b).

In one embodiment, the second dairy product is milk and/or streams derived from milk processing. Milk may be any kind of milk, such as skimmed milk, regular milk, whole milk, including ultrahigh temperature (UHT) treated milk and pasteurized milk. As mentioned previously, the dairy industry generates significant volumes of low-value side-streams, i.e. dairy waste. There is a great potential in transforming these waste materials into high value-added products. Milk derived streams may be any stream derived from milk production, such as a waste stream, for example a stream comprising whey, e.g. whey mother liquor.

Lactose is converted to glucose and galactose by the beta-galactosidase EC 3.2.1.23 enzyme. Several different bacteria naturally produce beta-galactosidase. When the method is used for reducing the lactose content in a dairy product, from a commercial/regulatory point of view, it is favorable to apply the method using a beta-galactosidase producing lactic acid bacterium, since lactic acid bacteria are naturally found in dairy products. Example 2 illustrates the performance of a selection of different nisin-permeabilized lactic acid strains comprising beta-galactosidase in hydrolyzing lactose. In one embodiment, the lactic acid bacterium comprising beta-galactosidase EC 3.2.1.23 may be selected from Streptococcus thermophilus, Lactobacillus casei, Lactobacillus plantarum, Lactobacillus helveticus, Lactobacillus delbrueckii, Lactobacillus acidophilus, and Lactococcus lactis. Preferably, the lactic acid bacterium is a S. thermophilus strain.

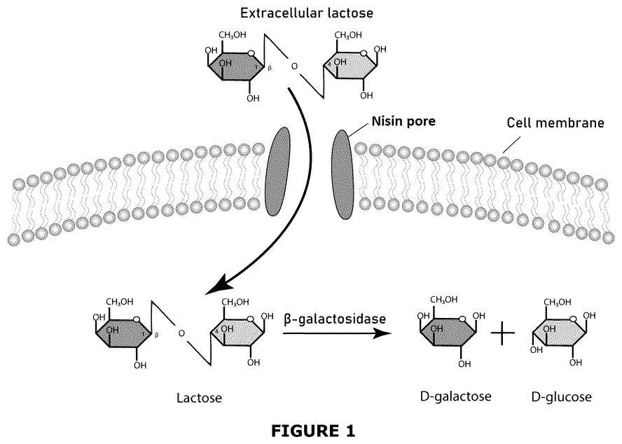

The lactic acid bacterium is incubated with nisin, such as with a nisin producing microbial cell culture and/or a culture medium derived thereof, under conditions easily optimized by a person skilled in the art by testing different combinations of temperature, time, cell concentration, etc. With their intended use in processing of a dairy product, the permeabilized cells are preferably harvested after nisin-treatment prior to adding them to the dairy product, and further optionally washed before adding them to the dairy product. Once added to the dairy product, the beta-galactosidase enzymes inside the cells will facilitate hydrolysis of lactose in a dairy product into glucose and galactose, yielding a lactose-reduced dairy product (see FIG. 1).

In one embodiment, the dairy product is a milk product, such as selected from skimmed milk, regular milk, whole milk, etc. Example 3 illustrates efficient hydrolysis of lactose in milk.

In one embodiment, the original lactose content of the dairy product is reduced by at least 50%, such as 55%, 60%, 65%, or even up to 70%. In one embodiment, 70% of the original lactose content of the dairy product is hydrolyzed by the nisin-permeabilized cells comprising beta-galactosidase, such as 75%, 80%, 85%, 90%, or even 95% or above is hydrolyzed.

In another embodiment, the method of the present invention is suitable for preparing yoghurt (or yoghurt-like products) having a reduced lactose content. The method described above for preparing a dairy product having reduced lactose content may in this regard be followed by the addition of a step:
e. culturing yoghurt starter bacteria in the product obtained in step (d), wherein said starting dairy product is milk.

The added yoghurt starter bacteria will thereby facilitate conversion of the lactose-reduced milk to lactose-reduced yoghurt.

Specifically, the steps for making a yoghurt product having a low lactose content may be performed by the method of the present invention comprising the steps of:
a. providing lactic acid bacteria comprising beta-galactosidase EC 3.2.1.23 for catalyzing conversion of lactose to galactose and glucose,
b. incubating said bacteria with nisin,
c. optionally harvesting permeabilized bacteria obtained in step (b),
d. incubating permeabilized bacteria obtained in step (b) or (c) with milk,
e. culturing yoghurt starter bacteria in the hydrolyzed milk product obtained in step (d).

Yoghurt starter bacteria are commonly known in the art, and for example may be selected from Streptococcus thermophilus and Lactobacillus deibruckii subsp. bulgaricus. In preparing lactose-reduced yoghurt, in one embodiment, the permeabilized cells comprising beta-galactosidase may hydrolyze between 50-100% of the lactose in milk, such as between 70-100% of the lactose. In another embodiment, the permeabilized cell catalyst may hydrolyze merely 50%, 55%, 60%, 65%, 70%, 75%, or 80% of the lactose prior to addition of the yoghurt starter culture, where after the remaining lactose content may be removed (utilized) by the yoghurt starter culture in combination with further hydrolysis by the permeabilized cell catalyst.

In a further embodiment, the method of the present invention may be applied to increase the sweetness of a lactose product. As a non-limiting example, partially hydrolyzed lactose may be used as a sweetener, as the presence of several types of sugars has a synergistic effect on sweetness. Thus the method can be used in all types of lactose-comprising products, such as yoghurts or even chocolates, to increase sweetness, and at the same time allow for reducing the overall added-sugar content.

In a further embodiment of the invention, value-added compounds may be produced. One such example is tagatose and fructose from the isomerization of galactose and glucose, respectively. This may be facilitated by microbial cells comprising arabinose isomerase EC 5.3.1.4 (for conversion of galactose to tagatose) and/or xylose isomerase EC 5.3.1.5 (for conversion of glucose to fructose).

In one embodiment, the method of the invention comprises treating a microbial cell with nisin as described herein, wherein the cell comprises (i) xylose isomerase EC 5.3.1.5 for conversion of glucose to fructose and/or (ii) arabinose isomerase EC 5.3.1.4 for conversion of galactose to tagatose; and wherein the substrate is glucose and/or galactose, respectively.

A microbial cell comprising arabinose isomerase EC. 5.3.1.4 and/or xylose isomerase EC 5.3.1.5 may in a preferred embodiment be selected from the group of lactic acid bacteria. These microbial cells may be added to a lactose-reduced milk product produced by the method of the invention. In a preferred embodiment, the microbial cell comprising arabinose isomerase EC 5.3.1.4 and/or xylose isomerase EC 5.3.1.5 is nisin-permeabilized, such as by methods described in section II for more efficient isomerization of the sugars.

In a further embodiment of the invention, the method may be applied to prepare a nisin-permeabilized bacterium comprising alpha-acetolactate decarboxylase EC 4.1.1.5 to be used for efficient conversion of alpha-acetolactate into acetoin.

In yet a further embodiment of the invention, the method may be applied to prepare a nisin-permeabilized bacterium comprising alpha-acetolactate synthase EC 2.2.1.6 to be used for efficient conversion of pyruvate into alpha-acetolactate—such as demonstrated in example 6.

In a further embodiment of the invention, microbial cells are treated with a combination of nisin and monolaurin for obtaining a further improved catalyst—such as demonstrate in example 7. More specifically, in step b of the method of the present invention, incubation of the microbial cell with nisin in combination with monolaurin may be beneficial.

Hence, the present invention also concerns a method for reducing the amount of a substrate in a sample, said method comprising the steps of:
a. providing microbial cells comprising at least one intracellular enzyme for catalyzing conversion of said substrate into one or more products,
b. incubating said microbial cells with nisin and monolaurin,
c. optionally harvesting permeabilized cells obtained in step (b),
d. incubating permeabilized cells obtained in step (b) or harvested cells obtained in step (c) with said sample comprising said substrate;
   wherein said microbial cells are susceptible to nisin-permeabilization, and wherein the substrate can transit through nisin pores of the permeabilized cells.

### IV. A Composition Comprising Nisin-Permeabilised Microbial Cells

One aspect of the present invention relates to a composition comprising (i) nisin-permeabilised microbial cells comprising an intracellular enzyme and (ii) substrate(s) and product(s) of a reaction catalyzed by said enzyme—as defined in previous sections of the present application.

In one embodiment, the composition comprises (i) nisin-permeabilized lactic acid bacteria comprising beta-galactosidase EC 3.2.1.23, and (ii) a dairy product comprising lactose and glucose and galactose.

In another embodiment, the composition comprises (i) nisin-permeabilized lactic acid bacteria comprising beta-galactosidase and a nisin-permeabilized microbial cell comprising arabinose isomerase EC 5.3.1.4 and/or xylose isomerase EC 5.3.1.5 and (ii) a dairy product comprising one or more of lactose, glucose, galactose, tagatose, and sucrose.

### V. a Nisin Producing Microbial Cell

One aspect of the present invention relates to a nisin producing microbial cell.

In one embodiment, the nisin producing microbial cell is derived from parent strain Lactococcus lactis subsp. lactis bv. diacetylactis SD96 (NCBI accession No. SRX6686433) by virtue of inserting transposon Tn5307 (SEQ ID NO. 4) comprising nisin biosynthesis gene cluster and genes needed for metabolizing sucrose into the genome of the parent strain.

In a further embodiment, the nisin producing microbial cell is phenotypically devoid of lactate dehydogenase activity.

In a further embodiment, the nisin producing microbial cell is further characterized by the ability to grow in milk at a temperature of 40° C., by virtue of the following genetic modifications in the genome when compared to the genome of the parent strain:
I. a parent gene encoding UDP-N-acetylmuramate-L-alanine ligase of SEQ ID No. 8 is modified to encode said amino acid sequence having substitution F68L,
II. a parent gene encoding GTP pyrophosphokinase (RelA) of SEQ ID No. 10 is modified to encode said amino acid having substitution V469L,
III. base pairs 1,823,878-1,897,135 (SEQ ID No. 11) in the parent genome are deleted, and
IV. a tandem repeat ((A)6 to (A)5) upstream of the CodY transcription regulator (SEQ ID No. 12) in the parent genome is deleted,

In a preferred embodiment, the nisin producing microbial cell of the invention for producing a microbial cell catalyst is strain Ge001.

### VI. Use of Nisin-Permeabilized Cells as Whole-Cell Catalyst

One aspect of the present invention concerns the use of nisin-permeabilized microbial cells comprising an intracellular enzyme as whole-cell catalyst in an enzyme reaction. An essential prerequisite of the invention is that the substrate(s) of the enzyme reaction catalyzed by the intracellular enzyme of the nisin-permeabilized microbial cells has a size/shape/conformation that allows their transit through nisin pores of the nisin-permeabilized microbial cells.

Any given organism susceptible to nisin-permeabilization comprising an intracellular enzyme having a substrate small enough to travel through the nisin pore may be of use in the present invention—the enzyme and its substrate being defined in greater detail in previous sections of the present application.

In one embodiment, the nisin-permeabilized microbial cells are lactic acid bacteria comprising beta-galactosidase for hydrolysis of lactose. The nisin-permeabilized cells are thereby used as whole-cell catalysts for lactose hydrolysis in e.g. dairy products. The beta-galactosidase of S. thermophilus or other lactic acid bacteria naturally contributes to the partial hydrolysis of lactose in dairy foods such as yogurt during product manufacture and again during the passage through the gastrointestinal tract, as the result of permeabilization by bile acids. Therefore, fortification of milk with permeabilized S. thermophilus prior to direct consumption or incorporation into milk-based products as a source of beta-galactosidase thereby provides a close to “natural process”, wherein the beta-galactosidase of inherently safe and edible S. thermophilus needs no further purification or isolation to qualify for food-grade status.

In one embodiment, the invention concerns the use of a nisin producing microbial cell as described herein for producing a microbial cell catalyst, preferably comprising an intracellular beta-galactosidase enzyme. In a preferred embodiment, the nisin producing microbical cell is derived from parent strain Lactococcus lactis subsp. lactis bv. diacetylactis SD96 (NCBI accession No. SRX6686433) by virtue of inserting a copy of transposon Tn5307 (SEQ ID NO. 4) comprising nisin biosynthesis gene cluster and genes needed for metabolizing sucrose into the genome of the parent strain. In a further preferred embodiment, the nisin producing microbical cell is further characterized by being devoid of lactate dehydrogenase activity. In a further preferred embodiment, the nisin producing microbial cell is further characterized by the ability to grow in milk at a temperature of 40° C., by virtue of the following genetic modifications in the genome when compared to the genome of the parent strain: (I) a parent gene encoding UDP-N-acetylmuramate-L-alanine ligase of SEQ ID No. 8 is modified to encode said amino acid sequence having substitution F68L, (II) a parent gene encoding GTP pyrophosphokinase (ReIA) of SEQ ID No. 10 is modified to encode said amino acid having substitution V469L, (III) base pairs 1,823,878-1,897,135 (SEQ ID No. 11) in the parent genome are deleted, and (IV) a tandem repeat ((A)6 to (A)5) upstream of the CodY transcription regulator (SEQ ID No. 12) in the parent genome is deleted. In a most preferred embodiment, the nisin producing microbial cell is strain Ge001.

In another embodiment, nisin-permeabilized cells may be used as whole-cell catalyst in producing value-added compounds. One example is a nisin-permeabilized microbial cell capable of conversion of glucose and galactose to fructose and tagatose.

### VII. A Method of Detecting Products Produced by the Nisin-Permeabilized Cells

Methods for detecting and quantifying products, such as sugars, produced by a microbial cell of the invention include high performance liquid chromatography (HPLC) combined with refractive index detection to identify and quantify the products compared to standards, as one ordinary skilled in the art would be familiar with. Example 1 comprises the outline of one method of detection and quantification of sugars.

### VIII. Advantages and Commercial Application

Nisin was first identified in 1928 in fermented milk cultures and commercially marketed in England in 1953 as an antimicrobial agent. In 1969, nisin was approved by the Joint Food and Agriculture Organization/World Health Organization (FAO/WHO) as a safe food additive. In the United States, nisin was approved by the Food and Drug Administration in 1988 and was given a generally regarded as safe (GRAS) designation for use in processed cheeses.

Nisin therefore represents an excellent choice as a permeabilizing agent—especially within the food industry as it is a natural compound, which is food approved and easy to handle. Further, in the present invention, only a very low concentration of nisin is required for permeabilizing the cells.

Using nisin-permeabilized cells as a substitute for commercial purified β-galactosidases has many advantages such as low cost, low resource utilization, and possibility for clean-label status of lactose-free dairy products. Also of importance, is that it provides a more natural process when compared to commercial lactase solutions, where lactases are often derived from GMO's (engineered microorganisms).

Somkuti et al. 1998, have previously demonstrated that ethanol-permeabilized lactic acid bacteria can be used as lactase, however, using ethanol has some obvious drawbacks, such as in terms of handling the ethanol, as large amounts of ethanol are involved and some ethanol could end up in the final product. Kosher/Halal status of dairy products is increasingly important, and using ethanol as a permeabilizing agent may raise issues in this respect, as small amounts of ethanol could be introduced, with the cells, into the product. Furthermore, it was demonstrated in Example 5 that ethanol compromised the stability of the whole-cell catalyst, hence more cells will be needed to achieve a satisfactory degree of hydrolysis within a certain time limit. Meanwhile, nisin-treated cells showed a higher stability. Summarized, Example 5 clearly shows that nisin-treated cells perform better as whole-cell catalysts than ethanol-treated cells.

Further, the high stability of nisin-permeabilized cells supports the re-use of them as cell catalyst. The cells may simply be harvested after use and re-used, which could further lower the cost of use compared to e.g. conventional purified enzymes that cannot simply be collected and reused.

In all of the above embodiments, nisin may be substituted by any pore forming lantibiotic, such as Class A lantibiotics including bisin, subtilin, epidermin, gallidermin, and mutacin.

Hence, the present invention concerns a method for reducing the amount of a substrate in a sample, said method comprising the steps of:
a. providing microbial cells comprising at least one intracellular enzyme for catalyzing conversion of said substrate into one or more products,
b. incubating said microbial cells with a Class A lantibiotic,
c. optionally harvesting permeabilized cells obtained in step (b),
d. incubating permeabilized cells obtained in step (b) or harvested cells obtained in step (c) with said sample comprising said substrate;
wherein said microbial cells are susceptible to nisin-permeabilization, and wherein the substrate can transit through nisin pores of the permeabilized cells.

The present invention further concerns a whole-cell catalyst comprising Class A lantibiotic-permeabilized microbial cells; wherein the permeabilized cells comprise at least one intracellular enzyme for catalyzing conversion of a target substrate; and wherein the permeabilized cells are in a frozen or dried state.

The present invention further concerns a composition comprising (i) Class A lantibiotic-permeabilised microbial cells comprising at least one intracellular enzyme and (ii) substrate(s) and product(s) of a reaction catalyzed by said at least one enzyme.

The present invention further concerns use of Class A lantibiotic-permeabilized microbial cells comprising at least one intracellular enzyme as whole-cell catalyst in an enzyme reaction, wherein substrate(s) and product(s) of said reaction can transit through Class A lantibiotic-pores of the permeabilized microbial cells.

### EXAMPLES

### Example 1: Nisin-Permeabilized S. thermophilus can Hydrolyze Lactose

### 1.1. Microbial Strains

Streptococcus thermophilus (S. thermophilus) strain CS1980 was isolated from a Danish yoghurt. Other S. thermophilus strains were isolated from starter cultures kindly provided by Sacco Sri. Italy (ST057, ST022).

### 1.2 Growth Media

S. thermophilus was grown at 37° C. in LM17 medium (Sigma-Aldrich, Darmstadt, Germany) further comprising 20% whey mother liquor (ML) and 10% HFI-110 in 10 ml test tubes, without agitation. ML is the concentrated residue remaining after extraction of lactose from whey. HFI-110 is a whey protein hydrolysate prepared by Aria Foods Ingredients.

### 1.3 Cell Permeabilization Using Nisin A

S. thermophilus CS1980 was grown in 500-ml Erlenmeyer flasks containing 200 ml of medium at 37° C. for 16 h without agitation. As inoculum, 2 ml of a 24 hour, outgrown culture was used. Cells were collected by centrifugation at 10,000 g for 10 min at 4° C., washed once with sterile POM buffer (50 mM K2HPO4/KH2PO4, 1 mM MgCl2, pH 7.4) and finally re-suspended in POM buffer to an optical density (600 nm) of 0.33, corresponding to approximately 100 mg/i dry cell weight. The cell suspension was kept on ice until use. For permeabilization, cell suspensions were dispensed into sterile tubes (1 ml per tube) and centrifuged in a microcentrifuge at top speed for 5 min at 4° C. After decanting supernatants and draining tubes on sterile paper towels, pellets were re-suspended in 1 ml nisin A (2.5 μg/ml, Sigma-Aldrich, Darmstadt, Germany) in POM buffer and held for 10 min at 28 or 37° C. cells were collected as before and kept on ice for later use. Controls included non-nisin treated cells.

### 1.4 Lactose Hydrolysis by Nisin-Permeabilized Cells

The nisin-permeabilized S. thermophilus cells (100 mg/i dry cell weight) were added to a 50 g/l lactose solution (POM buffer) and incubated at different temperatures for 72 hours. Samples were withdrawn at different time points, and conversion of lactose (formation of glucose and galactose) was determined by HPLC analysis.

### 1.5 Analysis of Sugar Products

The concentrations of sugars (carbohydrate monomers and dimers) were determined using an Ultimate 3000 high-pressure liquid chromatography system (Dionex, Sunnyvale, CA) equipped with an Aminex HPX-87H column (Bio-Rad, Hercules, CA) and a Shodex RI-101 detector (Showa Denko KK, Tokyo, Japan). The column oven temperature was set to 30° C., and the mobile phase consisted of 5 mM H2SO4 with a flowrate of 0.5 ml/min.

### 1.6 Hydrolysis Results

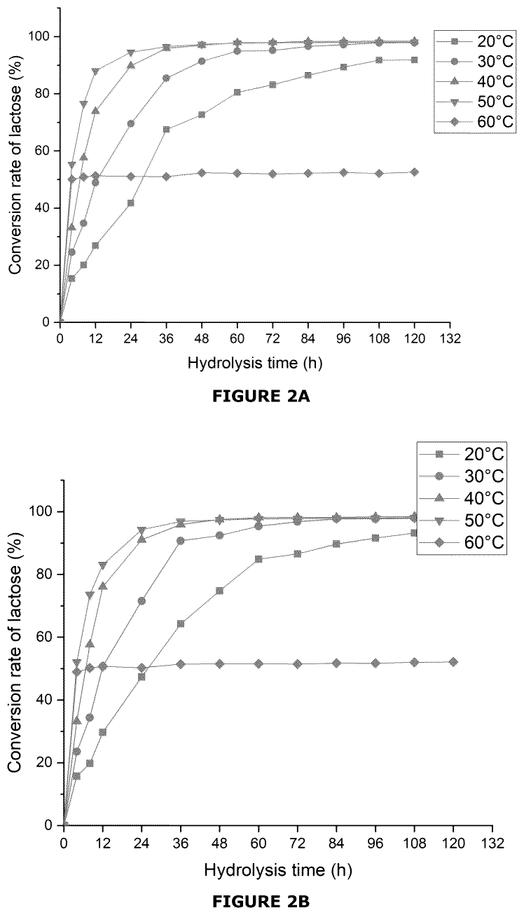

The cell catalyst was tested at different temperatures (20, 30, 40, 50, and 60° C.). It was found that the initial hydrolysis rate was fast at 60° C., but already after approx. 2 hours, the catalyst was inactivated. At lower temperatures, the catalyst was more stable. At 50° C., 95% of the lactose had been hydrolyzed within 24 hours (FIG. 2).

Comparing FIGS. 2A and 2B, it is seen that permeabilization may be performed within a wide temperature range: permeabilization at 28° C. and 37° C. seem to be equally efficient—it does not affect the final lactose conversion. Rather, it is the enzyme's stability that is challenged at the higher temperatures, resulting in the lower conversion at high hydrolysis temperature. Example 4 provides more examples demonstrating the influence of permeabilization temperature.

### Example 2: Comparison of Performance of Different Nisin-Permeabilized S. thermophilus Whole-Cell Catalysts

### 2.1. Microbial Strains, Nisin-Permeabilization and Lactose Hydrolysis

Streptococcus thermophilus (S. thermophilus) strain CS1980 was isolated from a Danish yoghurt (same as used in example 1). Additional S. thermophilus strains were isolated from starter cultures kindly provided by Sacco SrI. Italy (ST057, ST022).

The different S. thermophilus strains were nisin permeabilized, and tested for their ability to serve as whole-cell catalyst (lactase source) in lactose hydrolysis as described in example 1. Permeabilization of the cells was achieved using 2.5 μg/ml nisin in POM buffer for 10 minutes at 28° C. Hydrolysis was performed at 50° C.

### 2.1 Results

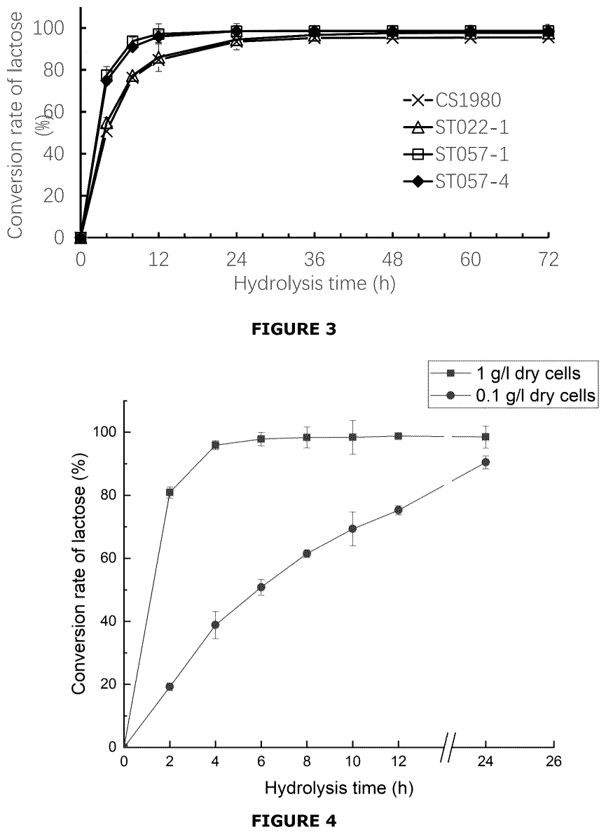

All isolates were able to hydrolyze lactose by the method of the present invention, see FIG. 3. The two strains from starter culture ST057 were almost twice as efficient as the other strains, facilitating >90% conversion of the lactose in less than half of the time.

### Example 3: Nisin-Permeabilized S. thermophilus can Hydrolyze Lactose in Milk

Above, the performance of the cell catalyst in buffered lactose solution has been tested, however, since the obvious substrate is milk or lactose-containing feedstocks derived from milk processing, it is relevant to characterize its performance in milk as well.

### 3.1. Microbial Strains, Nisin-Permeabilization and Lactose Hydrolysis

Nisin-treated S. thermophilus ST057-1 was applied in hydrolysis of lactose in milk, at cell concentration of approximately 100 mg/l or 1 g/l. Permeabilization of the cells was achieved using 2.5 μg/ml nisin in POM buffer for 10 minutes at 28° C. The starting concentration of lactose in the milk used was 52 g/l. Hydrolysis took place at 50° C.

### 3.2 Results

Hydrolysis of lactose in milk occurs slower than in POM buffer (FIG. 3 compared to FIG. 4): using 100 mg/l whole-cell catalyst concentration, 80% hydrolysis can be reached in within 4 hours POM buffer, whereas more than 12 hours is needed in milk.

Though milk can generally tolerate elevated temperatures (such as 50° C.) for an extended period of time without affecting its quality, a more rapid hydrolysis is preferred. Increasing the whole-cell catalyst concentration, the conversion rate was significantly increased (FIG. 4), reaching conversion levels as seen in the buffered samples.

### Example 4: Nisin-Permeabilization is Efficient Over a Broad Temperature Range

### 4.1. Microbial Strains, Nisin-Permeabilization and Lactose Hydrolysis

S. thermophilus CS1980 was nisin-permeabilized using 2.5 μg/ml nisin in POM buffer for 10 or 30 minutes at 28, 50 and 55° C. Controls were prepared without addition of nisin in the permeabilization step. Hydrolysis of 50 g/l lactose in POM using nisin-treated cells was carried out at a cell concentration of approximately 100 mg/l. Hydrolysis took place at 50° C.

S. thermophilus ST057-1 was nisin-permeabilized using 2.5 μg/ml nisin in POM buffer for 10 minutes at 28° C. or 37° C. Hydrolysis of lactose in milk (52 g/I lactose) using nisin-treated cells was carried out at a cell concentration of approximately 1 g/l. Hydrolysis took place at 50° C.

### 4.2. Results

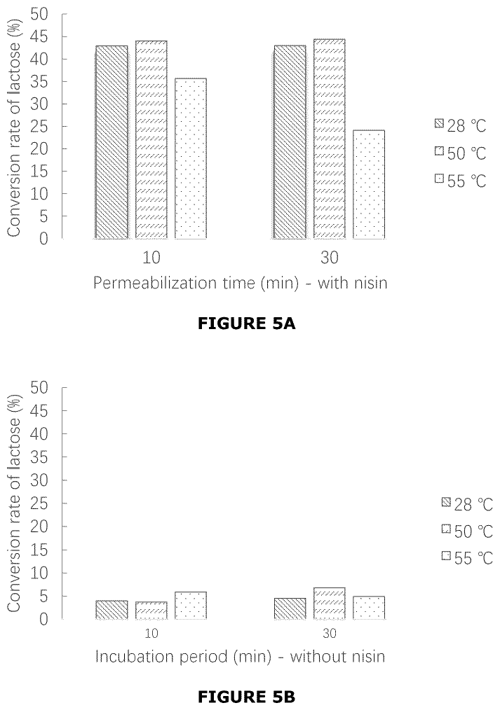

As seen in FIG. 5A, a broad range of permeabilization temperatures: 28-50° C., were found to be equally efficient. Temperatures as high as 50° C. may be applied, but at 55° C. the cell stability (and likely also the enzyme stability, especially at the longer permeabilization time) seemed to be compromised, hence for this particular strain and enzyme combination it is undesirable to use such high temperatures for the permeabilization. The control, where nisin was not added in the permeabilization step, showed only some very limited conversion (FIG. 5B); it is thereby clear that the temperature treatment itself is not the contributing cause of the substrate conversions reported herein. The nisin treatment is essential.

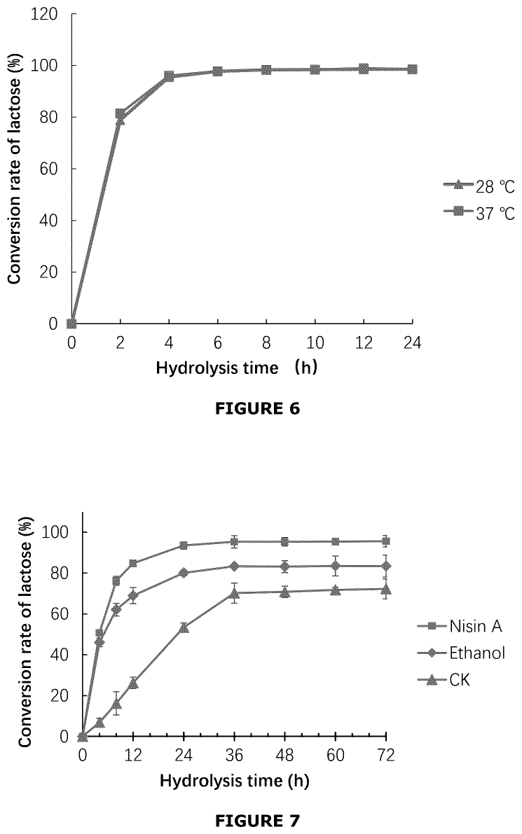

Further, as seen in FIG. 6, the different permeabilization temperature (28° C. vs 37° C.) provided nisin-permeabilized cells which performed equally well in terms of lactose conversion in milk samples.

FIG. 5A also demonstrates that prolonged nisin treatment (30 minutes vs 10 minutes) does not give better results. The nisin reaction is essentially instantaneous as long as the contraction of nisin is well-matched with the cell concentration.

### Example 5: Nisin-Permeabilized Cells Perform Better than Ethanol-Permeabilized Cells

Somkuti et al. 1998, previously demonstrated that ethanol-permeabilized lactic acid bacteria can be used as a source of lactase activity. The present example compared the present invention to the current state of the art.

### 5.1. Microbial Strains, Permeabilization and Lactose Hydrolysis

S. thermophilus CS1980 was permeabilized for 10 minutes at 28° C. using (i) 2.5 μg/ml nisin in POM buffer or (ii) 45% (v/v) ethanol for 10 minutes, or (iii) not permeabilized (control). Hydrolysis of lactose in POM buffer (50 g/I lactose) at 50° C. was carried out at a cell concentration of approximately 100 mg/l.

### 5.2. Results

As seen in FIG. 7, nisin-permeabilized cells perform better than the control as well as the ethanol-permeabilized cells in terms of lactose conversion. Ethanol is generally known to affect the stability of enzymes, which is supported by the data in FIG. 7: it was found that ethanol compromised the stability of the whole-cell catalyst, which is unfavorable in terms of cost, as more cells will be needed to achieve a satisfactory degree of hydrolysis within a certain time limit.

### Example 6: Nisin-Permeabilized L. lactis Catalyst for Conversion of Pyruvate to Alpha-Acetolactate

A modified L. lactis strain MG1363 lacking alpha-acetolactate decarboxylase activities was used in the present experiment. More specifically, the strain RD04 was L. lactis strain MG1363 ΔldhB ΔldhX Δpta ΔadhE ΔbutBA ΔaldB, while harboring an expression vector for overexpressing native alpha-acetolactate synthase (Als). RD04 was nisin-treated and tested for alpha-acetolactate synthase acticity in the present study as follows:
Cells were harvest in stationary phase (overnight culture),
resuspended to OD(600 nm)=1.0 in POM buffer containing 100 μg/ml nisin,
permeabilized by incubating for 15 min at 37° C.,
supernatant was removed,
cells were resuspended to OD(600 nm)=1 (0.36 g cells/l dry weight basis) in reaction solution (200 mM sodium pyruvate, 100 mM KPO₄ pH 6.0, 20 mM MgCl₂, 2 mM thiamine pyrophosphate).

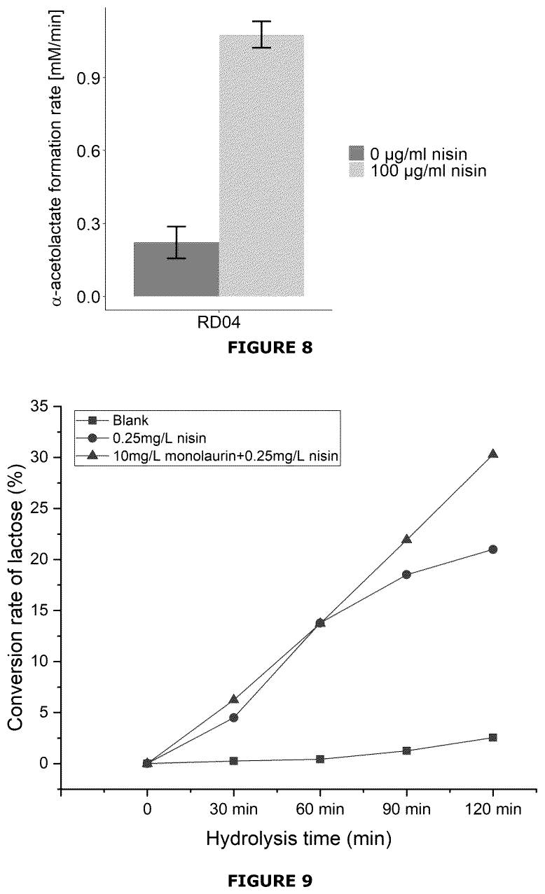

Alpha-acetolactate synthase activity was determined by investigating the conversion rate of pyruvate to alpha-acetolactate, using HPLC measurements of alpha-acetolactate formation. The results are reported in FIG. 8, showing that nisin permeabilized cells performed much better than non-permeabilized cells in conversion of pyruvate to alpha-acetolactate.

### Example 7: Nisin in Combination with Monolaurin Provides an Improved Catalyst

### 5.1. Microbial Strains, Permeabilization and Lactose Hydrolysis

S. thermophilus CS1980 (approximately 100 mg/l) was permeabilized in POM buffer pH 7.4 for 10 minutes at 28° C. using (i) 0.25 μg/ml nisin, (ii) 0.25 μg/ml nisin+10 μg/ml monolaurin, or (iii) not permeabilized (control—no addition of nisin or monolaurin). Hydrolysis of lactose (50 g/1) was carried out in POM buffer at 50° C.

### 5.2. Results

As seen in FIG. 9, nisin treatment in combination with monolaurin gives improved conversion rates of lactose compared to using nisin alone.

### Example 8: Nisin-Producing Microbial Strain Ge001

Parent strain: dairy isolate L. lactis subsp. lactis biovar diacetylactis SD96, which grows well in milk, and is generally insensitive to phage attack (Dorau et al 2020). A natural (non-engineered) approach using a combination of adaptive laboratory evolution (ALE) in milk at high temperatures, random mutagenesis using proflavine as mutagen, and finally conjugation for transfer of the nisin gene cluster, was applied.

L. lactis subsp. lactis biovar diacetylactis SD96 was cultivated on M17 agar supplemented with 0.5% lactose (LM17), and a single colony was used to start two separate cultures in 9 ml UHT milk. Generally, when the milk was coagulated, the culture was considered fully grown with approximately 10¹⁰ cells. Fully-grown cultures were homogenized by shaking and propagated in 9 ml fresh UHT milk. Then the procedure was repeated. Every week, the culture was saved by mixing a fully-grown culture with 50% glycerol 1:1 and storing at −80° C.

The culture was kept continuously at high temperatures. For avoiding a long time between propagation steps, the coagulated, fully grown culture was propagated by diluting 10-fold into 9 ml fresh UHT-milk, which corresponds to 3.32 generations per propagation step. Initially, SD96 was grown at 39° C., and the coagulation of the UHT milk was observable after approximately 48 h. 20 propagation steps were conducted until coagulation was observable after ca. 24 h, then the temperature was increased to 40° C., again resulting in coagulation after ca. 48 h. After 25 propagation steps at 40° C. (45 propagation steps in total, 150 generations), coagulation was observable after 24 h.

A first mutant strain was isolated after these 150 generations, approximately five months after the ALE was started. This first mutant strain was able to grow in milk and had improved thermotolerance compared to strain SD96.

A single colony of this first mutant stain grown on LM17-Agar was inoculated into 5 mL of M17 with 1% lactose (LM17) in a 20 mL test tube, put in a 45⁰ angled test tube rack, and cultivated at 30° C. and 220 rpm shaking. The overnight culture was diluted with fresh LM17 medium supplemented with 10 mg/L of proflavine to a final cell density (OD600) of 0.1. After 18 h incubation at 30° C. with shaking, the cells were harvested by centrifugation (5000 g for 2 min) and washed three times with 0.9% NaCl. The cells were then resuspended in fresh LM17 medium and incubated at 30° C. with shaking for 1 h. After appropriate dilution in 0.9% NaCl, the cells were plated on TTC medium to obtain single colonies.

TTC (2,3,5-triphenyltetrazolium chloride) is reduced to the red compound triphenylformazan under non-acidic conditions, and colonies which do not form acid (here lactate) appear as dark red on such plates.

By screening a large number of colonies on this TTC medium a second mutant with reduced LDH (lactate dehydrogenase) activity was obtained.

Finally, Ge001 was obtained after transferring a nisin gene cluster from the donor L. lactis ATCC 11454, by conjugation, into the lactate dehydrogenase (LDH) deficient second mutant.

The nisin gene cluster as well as the sucrose fermentation genes are located on a conjugative transposon (SEQ ID No. 4) in L. lactis (Broadbent et al 1995); this transposon can ‘jump’ from one strain and ‘insert’ itself into other L. lactis strains by conjugal transfer.

Solid-surface conjugation between donor ATCC 11454 and the mutant recipient was conducted using the method described by Broadbent et al 1991.

L. lactis is more resistant to nisin after introducing the nisin immunity gene (nisI), which is part of the nisin biosynthesis gene cluster. Nisin was therefore used to select for the desired transconjugant, in combination with the fact that only the mutant (not ATCC 11454) can grow on lactose.

Cells from solid surface milk agar were harvested in 1 ml of 0.85% saline and then 0.1 mL volumes were plated onto SA selective agar plates with 25 μg/mL nisin, 0.1% TTC (2,3,5-triphenyltetrazolium chloride) and 0.5% lactose, and incubated at 30° C. for 48 h. Plates were examined for red colonies.

SA medium: 1% (wt/vol) nonfat milk, 0.25% milk protein-hydrolysate peptone, 0.5% dextrose, and 1.5% agar. The pH was adjusted to 6.6, the agar medium was sterilized, and tempered at 45° C. after sterilization. Two solutions, one containing 10% potassium ferricyanide and one containing 1 g of ferric citrate and 1 g of sodium citrate in 40 ml of water, were steamed (100° C.) for 30 min. Ten milliliters of each solution was added to 1 liter of agar medium, and the agar was swirled gently and poured. Plates were dried in the dark for 24 h at 300 C.

Transconjugant Ge001 was picked up from the SA selective agar plates and analyzed. Sequencing revealed that a single copy of Tn5307, with a size of 66040 bp, was present in the genome of Ge001.

Of high relevance to the present invention, Ge001 is phenotypically characterized as follows: (i) grows well in milk, even at elevated temperatures up to 40° C., (ii) is deficient in lactate dehydrogenase activity, and (iii) both produces and tolerates nisin.

### Genotype of Ge001:

With regards to the genetic alterations related to good growth in milk at elevated temperatures, compared to the parent strain Lactococcus lactis subsp. lactis bv. diacetylactis SD96 (NCBI accession No SRX6686433), the genotype of Ge001 was found to comprise the following changes:
I. the parent gene encoding UDP-N-acetylmuramate-L-alanine ligase (SEQ ID No. 8) is modified to encode the amino acid sequence substitution F68L,
II. the parent gene encoding GTP pyrophosphokinase (RelA) (SEQ ID No. 10) is modified to encode the amino acid substitution V469L,
III. base pairs 1,823,878-1,897,135 (SEQ ID No. 11) in the parent genome are deleted, and
IV. the tandem repeat ((A)6 to (A)5) upstream of the CodY transcription regulator (SEQ ID No. 12) in the parent genome is deleted,

Further, sequencing revealed that the lactate dehydrogenase gene of Ge001 was disrupted by insertion of eight nucleotides in the Idh gene; more specifically insertion of CCGTCAAG in the CDS region of the Idh gene encoding lactate dehydrogenase (SEQ ID No. 5) between nucleotides T464 and C465

And finally, as specified above, the nisin gene cluster was introduced as a single copy of transposon Tn5307 (SEQ ID No. 4) in the genome of Ge001.

### Example 9: Ge001 Produces Nisin Using Dairy Waste as Fermentation Substrate

The ability of Ge001 to produce nisin was tested using dairy waste as fermentation substrate, where biomass accumulation, lactose consumption, citrate consumption, lactate formation, pH, and nisin production were monitored over time. Cell density was determined by measuring the optical density (OD) at a wavelength of 600 nm using UV-1600PC spectrophotometer (VWR, Denmark). The pH change during the growth was measured using a pH meter (Lab845, SI Analytics, Denmark). Nisin production was assayed using the bioassay agar plate diffusion method described below. Quantification of lactose, lactate, citrate was done using high-performance liquid chromatography. Nisin activity assay: To precisely estimate the nisin activity in the fermentation broth, the hot extraction method described by Zhang et al 2014 was used. The nisin activity was determined using the bioassay agar plate diffusion method and Micrococcus luteus ATCC 10240 as indicator strain as described by Thunyarat [33]. Briefly, a series of standard solutions containing 10-1,000 IU mL-1 were prepared by diluting a stock solution with 0.02 N HCl, and these were used to prepare a standard curve. Subsequently, the autoclaved NB agar medium was cooled to 40° C., and inoculated with 1% of M. luteus ATCC 10240 with OD600 2.0 (diluted using NB broth). The 25 mL medium was then poured into a sterile Petri dish and five 6 mm diameter wells were introduced after solidification. Eighty microliters of standard solution and test solutions were added to the wells. First, the agar plates were stored at 4° C. for 12 h to allow nisin to diffuse, and then the plates were transferred to a 30° C. incubator and kept there for 24 h. The diameters of the inhibition zones were measured horizontally and vertically using a digital caliper. The assay was done in triplicates.

Whey Mother liquor (ML), the residue obtained after lactose crystallization from concentrated whey permeate, was provided by Aria Food Ingredients (Viby J, Denmark), and its composition is described in Liu et al 2005. ML in combination with yeast extract can serve as a complete fermentation medium for L. lactis. Specifically, the nisin-producing strain Ge001 was streaked on an LM17-TTC plate, and incubated overnight at 30° C. A red single colony was inoculated into 25 mL of medium consisting of 10% ML and 1% yeast extract (YE) in a 250 mL shake flask and cultivated to early exponential phase. The 10% (v/v) seed-culture was inoculated into 25 mL of the same medium in a 250 mL shake flask culture.

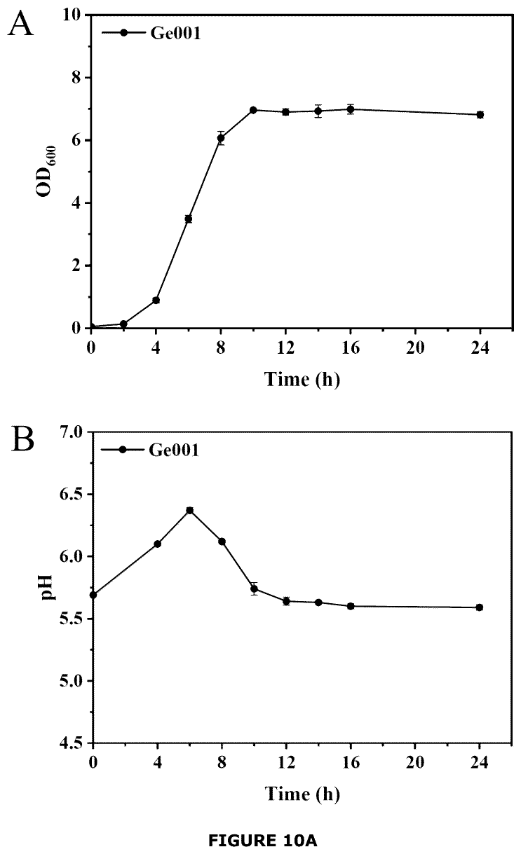

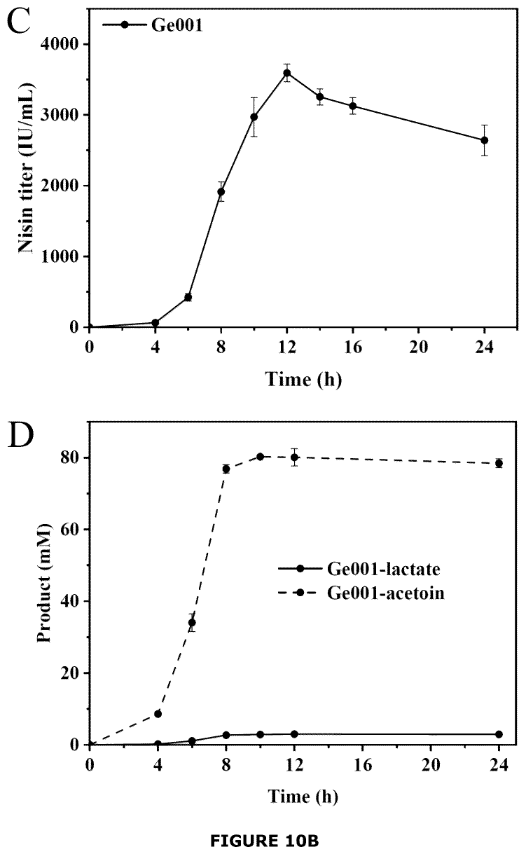

Ge001 grew well and had the ability to produce nisin in ML (FIGS. 10A and 10C). Nisin production by Ge001 was shown to be directly linked to growth, and nisin production ceased after entry into the stationary phase. After 12 hours, the nisin concentration decreased gradually over time, which most likely is due to proteolytic degradation and adsorption of nisin onto producer cells. During fermentation, only a small amount of lactic acid was formed, and the lactose was fully consumed (FIG. 10D). The pH increase observed in the first six hours was due to citrate consumption, however, the pH dropped subsequently to 5.5 to 6.5, which is suitable for growth and nisin production (FIG. 10B).

Further optimization of nisin production was investigated. It was found that both biomass and nisin titer (IU/mL) increased when increasing amounts of yeast extract (YE) were added. The highest nisin activity observed was 5003 IU/mL, when 2% YE was added. Ge001 needs to be cultured aerobically as oxygen is required by the NADH oxidase NoxE, the function of which is essential for Ge001 to grow efficiently without forming acidic products. One drawback of this is that oxidative stress can arise, which affects the growth of Ge001 negatively. Aerobic growth leads to formation of reactive oxygen species (ROS) with high oxidizing potential, and these can damage various cell constituents. Another drawback is that oxygen can disrupt the structure of nisin, and lead to loss of antimicrobial activity. L. lactis is able to respire when heme is added into the growth medium and there are studies that show the protective effect of heme against oxidative stress through elimination of ROS (Kaneko et al 1990). Different concentration of hemin (a chloride of heme) were added into the fermentation medium. The biomass and the nisin titer both increased after adding 0.5 to 2 μg/ml hemin into the fermentation medium. The addition of 1 μg/ml hemin resulted in the highest increase in biomass and nisin production after a 12 h cultivation. As such, the highest OD600 achieved in the hemin-stimulated fermentation was 17.6, while the highest nisin titer was 7416 IU/mL.

It was further tested if Mn2+ could have a beneficial effect on nisin production by further decreasing oxidative stress. It was found that Mn2+ can significantly promotes biomass and nisin production: When adding 0.5 or 1 mM Mn2+, the nisin titer could be increased by 21%.

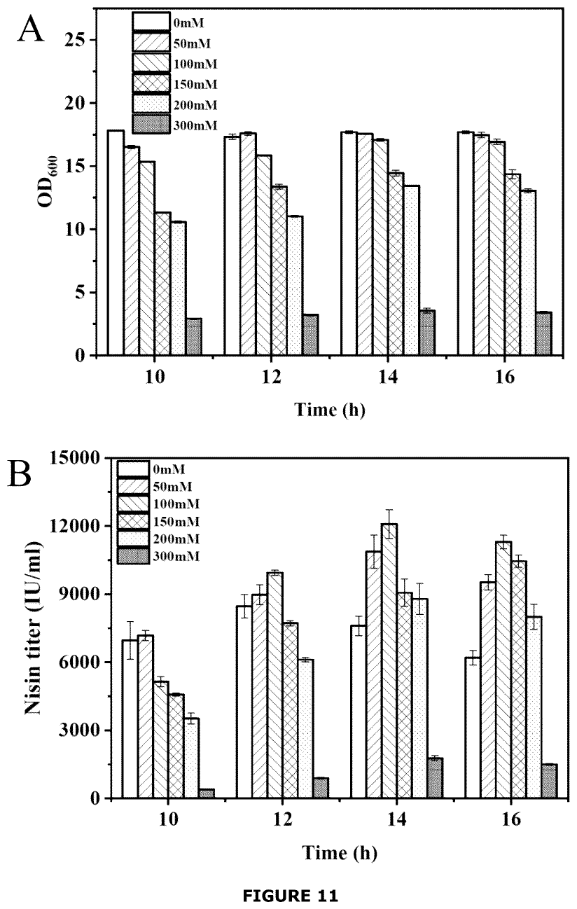

During the fermentation period, nisin were reversibly adsorbed onto the cells due to its positively-charged property. It was found that Ca2+ could displace nisin bound to the cell wall and thereby result in higher nisin titers. As shown in FIG. 11, addition of 0.05-0.2 M CaCl₂ led to an increase in biomass and nisin activity, however, when CaCl₂ exceeded 0.2 M there was significant growth inhibition. The nisin titer could reach the highest value (12,084 IU/mL) at 12 h of cultivation when 100 mM CaCl₂ was added.

### Example 10: Permeabilization of Microbial Cell Catalysts and Hydrolysis of Lactose in Milk Using the Nisin-Producing Strain Ge001

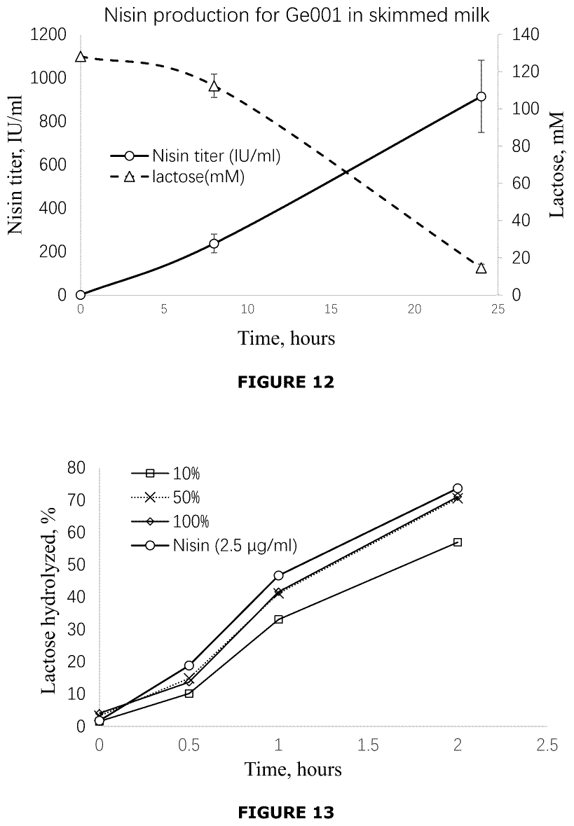

Ge001 was grown in 25 mL UHT (ultra high temperature treated) milk (1.5% fat content) using 300 mL shake flasks. Nisin production and lactose concentration was measured over a time course of 24 hours. As seen in FIG. 12, Ge001 is able to grow in UHT milk, utilize lactose, and produce nisin.

S. thermophilus ST057-4 was cultivated in LM17 (2% lactose) for 16 hours at 37° C. Cells were harvested by centrifugation at 4° C. and washed in cold POM buffer. Cells were resuspended in 1 ml (OD600 2.5/ca. 0.9 g/L) of (1) POM buffer with 2.5 μg/ml nisin, (2) milk cultured with Ge001 (100%), (3) two times diluted milk cultured with Ge001 (50%), or (4) ten times diluted milk cultured with Ge001 (10%). All dilutions of nisin culture were done using POM buffer. The cells were incubated for 30 min at 30° C. Afterwards, the cells were harvested by centrifugation, resuspended in 1 ml fresh UHT milk (1.5% fat), and incubated at 50° C. Samples were withdrawn regularly (0 h, 0.5 h, 1 h, and 2 h) and analyzed using HPLC. Before HPLC measurement, the milk samples were diluted 10-fold in a 1 M H2SO4 solution (final concentration) to clear the samples. The experiment was carried out using three independent replicates.

As can be seen in FIG. 13, the Streptococcus thermophilus cells permeabilized using Ge001 culture medium are able to hydrolyze lactose in milk.

### REFERENCES

Somkuti, G. A., Dominiecki, M. E., Steinberg, D. H. 1996. Sensitivity of Streptococcus thermophilus to Chemical Permeabilization. Current Microbiology 32:101-105.
Somkuti, G. A., Dominiecki, M. E., Steinberg, D. H. 1998. Permeabilization of Streptococcus thermophilus and Lactobacillus delbrueckii subsp. bulgaricus with ethanol. Curr. Microbiol. 36(4):202-6.
Dorau et al 2020. Complete genome sequence of Lactococcus lactis subsp. lactis bv. Diacetylactis SD96. ASM Microbiology resource announcements 9(3) e01140-19. DOI: 10.1128/MRA.01140-19
Broadbent et al 1995. Characteristics of Tn 5307 exchange and intergeneric transfer of genes associated with nisin production. Appl Microbiol Biotechnol 44, p 139-146. Doi: 10.1007/BF00164493
Broadbent et al 1991. Genetic construction of nisin-producing Lactococcus lactis subsp. cremoris an analysis of a rapid method for conjugation. Appl Environ Microbiol 57, p.517-524.
Zhang et al 2014. Genome shuffling of L. lactis subs. Lactis YF11 for improving nisin Z production and comparative analysis. J Dairy Sci. Doi: 10.3168/jbs.2013-7238.
Liu et al 2005. Effect of nutrient supplements on simultaneous fermentation of nisin and lactic acid from cull potatoes. Appl Biochem Biotechnol Part A Enzyme eng. And biotechnol. Pp 475-483. Doi:10.1007/978-1-59259-991-2_42.
Kaneko et al 1990. Acetoin fermentation by citrate-positive L. lactis subsp. lactis 302 grown aerobically in the presence of hemin or Cu2+. Appl Environ Mocrobiol 56; 2644-2649. Doi: 10.1128/aem.56.9.2644-2649.1990.

### Items of the Invention

1. A method for reducing the amount of a substrate in a sample, said method comprising the steps of:
   I. providing microbial cells comprising at least one intracellular enzyme for catalyzing conversion of said substrate into one or more products,
   II. incubating said microbial cells with nisin,
   III. optionally harvesting permeabilized cells obtained in step (b),
   IV. incubating permeabilized cells obtained in step (b) or harvested cells obtained in step (c) with said sample comprising said substrate;
      wherein said microbial cells are susceptible to nisin-permeabilization, and wherein the substrate-can transit through nisin pores of the permeabilized cells.
2. The method according to item 1, wherein said microbial cells provided in step (a) are bacteria selected from Escherichia, Streptococcus, Lactobacillus, Lactococcus, Lactovum, Pediococcus, Leuconostoc, Fructobacillus, Weissella, Oenococcus, Corynebacterium, Brevibacterium, Bacillus, Sporolactobacillus, Geobacillus, Halobacillus, Halolactibacillus, Tetragenococcus, Acetobacter, Acinetobacter, Proprionibacterium, and Bifidobacterium.
3. The method according to item 1 or 2, wherein nisin is added in the form of a nisin producing microbial cell or a culture medium derived from a nisin producing microbial cell.
4. The method according to item 3, wherein the nisin producing microbial cell is a lactic acid bacterium, such as a Lactococcus species.
5. The method according to any one of items 1-4, wherein the sample is a food or beverage.
6. The method according to any one of items 1-5, for reducing the lactose content of a dairy product, said method comprising the steps of:
   I. providing cells of a lactic acid bacterium comprising intracellular beta-galactosidase EC 3.2.1.23. for catalyzing conversion of lactose to galactose and glucose,
   II. incubating said cells of (a) with nisin,
   III. optionally harvesting permeabilized cells obtained in step (b),
   IV. incubating permeabilized cells obtained in step (b) or (c) with said dairy product.
7. The method according to item 6, wherein the lactic acid bacterium is selected from among Streptococcus thermophilus, Lactobacillus casei, Lactobacillus plantarum, Lactobacillus helveticus, Lactobacillus delbrueckii, Lactobacillus acidophilus, and Lactococcus lactis.
8. The method according to item 6 or 7, wherein the dairy product is a milk product, such as skimmed milk, regular milk, whole milk, yoghurt (and yoghurt like products, e.g. Gaio®, Cultura®), Skyr, Quark, Greek yoghurt, butter milk, cream, whey and butter.
9. The method according to any one of items 6-8, wherein the permeabilized cells are harvested in step (c); wherein the dairy product in step (d) is milk, and wherein said method further comprises the step of:
   e. culturing cells of a yoghurt starter bacterium in the product obtained in step (d).
10. The method according to item 9, wherein the yoghurt starter bacterium is Streptococcus thermophilus or Lactobacillus delbruckii subsp. bulgaricus.
11. The method according to any one of items 6-10, wherein in step (d) said dairy product is additionally incubated with nisin-permeabilized microbial cells comprising (i) xylose isomerase EC 5.3.1.5 for conversion of glucose to fructose. and/or (ii) arabinose isomerase EC 5.3.1.4 for conversion of galactose to tagatose.
12. A whole-cell catalyst comprising nisin-permeabilized microbial cells; wherein the nisin-permeabilized cells comprise at least one intracellular enzyme for catalyzing conversion of a target substrate; and wherein the nisin-permeabilized cells are in a frozen or dried state.
13. A composition comprising (i) nisin-permeabilised microbial cells comprising at least one intracellular enzyme and (ii) substrate(s) and product(s) of a reaction catalyzed by said at least one enzyme.
14. Use of nisin-permeabilized microbial cells comprising at least one intracellular enzyme as whole-cell catalyst in an enzyme reaction, wherein substrate(s) and product(s) of said reaction can transit through nisin pores of the nisin-permeabilized microbial cells.
15. The use according to item 14 wherein said nisin-permeabilized microbial cells are bacteria comprising beta-galactosidase, wherein said substrate is lactose.

1. A method for reducing the amount of a substrate in a sample, said method comprising the steps of:
(a) providing one or more non-GMO microbial cells comprising at least one intracellular enzyme for catalyzing conversion of said substrate into one or more products,
(b) incubating said microbial cells with nisin,
(c) optionally harvesting permeabilized cell catalysts obtained in step (b),
(d) incubating permeabilized cell catalysts obtained in step (b) or step (c) with said sample comprising said substrate;
wherein said microbial cells are susceptible to nisin-permeabilization, and wherein the substrate can transit through nisin pores of the permeabilized cell catalysts.

2. The method according to claim 1, wherein the non-GMO microbial cell is a lactic acid bacterium, the substrate is lactose, the sample is a first dairy sample, the intracellular enzyme is beta-galactosidase, and the products are glucose and galactose.

3. The method according to claim 2, wherein the lactic acid bacterium is selected from the list consisting of Streptococcus thermophilus, Lactobacillus casei, Lactobacillus plantarum, Lactobacillus helveticus, Lactobacillus delbrueckii, Lactobacillus acidophilus, and Lactococcus lactis.

4. The method according to claim 1, wherein nisin is present in a concentration of 2-250 μg nisin per mg cells.

5. The method according to claim 1, wherein nisin is added in the form of a nisin producing microbial cell or a culture medium derived from a nisin producing microbial cell; preferably wherein the nisin producing microbial cell is a non-GMO strain.

6. The method according to claim 5, wherein said nisin producing microbial cell culture is obtained by culturing one or more nisin producing microbial cells in a second dairy sample, prior to incubating with said non-GMO microbial cell in step (b).

7. The method according to claim 5, wherein the nisin producing microbial cell comprises a transposon Tn5307 (SEQ ID NO. 4) comprising nisin biosynthesis gene cluster and genes needed for metabolizing sucrose is inserted into the parent genome

8. The method according to claim 5, wherein the nisin producing microbial cell is a lactic acid bacterium, preferably a Lactococcus species.

9. The method according to claim 5, wherein the nisin producing microbial cell is phenotypically characterized as deficient in lactate dehydrogenase activity.

10. The method according to claim 5, wherein the nisin producing microbial cell is derived from parent strain Lactococcus lactis subsp. lactis bv. diacetylactis SD96 (NCBI accession No. SRX6686433) by virtue of the following genome modifications in the genome when compared to the genome of the parent strain:
(i) a transposon Tn5307 (SEQ ID NO. 4) comprising nisin biosynthesis gene cluster and genes needed for metabolizing sucrose is inserted into the parent genome,
(ii) nucleic acid sequence CCGTCAAG is inserted into the CDS region of the parent Idh gene (SEQ ID No. 5) encoding lactate dehydrogenase between nucleotides T464 and C465,
(iii) a parent gene encoding UDP-N-acetylmuramate-L-alanine ligase of SEQ ID No. 8 is modified to encode said amino acid sequence having substitution F68L,
(iv) a parent gene encoding GTP pyrophosphokinase (ReIA) of SEQ ID No. 10 is modified to encode said amino acid having substitution V469L,
(v) base pairs 1,823,878-1,897,135 (SEQ ID No. 11) in the parent genome are deleted, and
(vi) a tandem repeat ((A)6 to (A)5) upstream of the CodY transcription regulator (SEQ ID No. 12) in the parent genome is deleted.

11. The method according to claim 5, wherein the nisin producing microbial cell is Lactococcus lactis subsp. lactis bv. diacetylactis SD96 strain Ge001 deposited with depository institution DSMZ German collection of microorganism and cell cultures.

12. The method according to claim 2, wherein the first dairy sample is a milk product selected from the list consisting of skimmed milk, regular milk, whole milk, yoghurt, Skyr, Quark, Greek yoghurt, butter milk, cream, whey, and butter.

13. The method according to claim 2, wherein the permeabilized cell catalysts are harvested in step (c); wherein the first dairy sample is milk, and wherein said method further comprises the step of:
(e) culturing cells of a yoghurt starter bacterium, preferably Streptococcus thermophilus or Lactobacillus delbruckii subsp. bulgaricus, in the products obtained in step (d).

14. A whole-cell catalyst comprising nisin-permeabilized microbial cells or a composition comprising said whole-cell catalyst; wherein the microbial cells were permeabilized with nisin at a concentration of 2-250 μg nisin per mg cells; and further wherein the nisin-permeabilized cells are in a frozen or dried state.

15. The whole-cell catalyst or composition according to claim 14, wherein the nisin-permeabilized cells comprise an intracellular beta-galactosidase enzyme for catalyzing conversion of lactose into glucose and galactose.

16. A nisin producing microbial cell derived from parent strain Lactococcus lactis subsp. lactis bv. diacetylactis SD96 (NCBI accession No. SRX6686433) by virtue of inserting transposon Tn5307 (SEQ ID NO. 4) comprising nisin biosynthesis gene cluster and genes needed for metabolizing sucrose into the genome of the parent strain.

17. The nisin producing microbial cell according to claim 16 phenotypically devoid of lactate dehydogenase activity.

18. The nisin producing microbial cell according to claim 16, further characterized by the ability to grow in milk at 40° C. temperatures, by virtue of the following genetic modifications in the genome when compared to the genome of the parent strain:
(I) a parent gene encoding UDP-N-acetylmuramate-L-alanine ligase SEQ ID No. 8 is modified to encode said amino acid sequence having substitution F68L,
(II) a parent gene encoding GTP pyrophosphokinase (ReIA) SEQ ID No. 10 is modified to encode said amino acid sequence having substitution V469L,
(III) base pairs 1,823,878-1,897,135 (SEQ ID No. 11) in the parent genome are deleted, and
(IV) a tandem repeat ((A)6 to (A)5) upstream of the CodY transcription regulator (SEQ ID No. 12) in the parent genome is deleted,

19. The nisin producing microbial cell according to claim 16, wherein the nisin producing microbial cell is Lactococcus lactis subsp. lactis bv. diacetylactis SD96 strain Ge001 deposited with depository institution DSMZ German collection of microorganism and cell cultures.


SEQUENCE LISTING (12 sequences)

SEQ ID NO: 1 - DNA, 174 bp, Lactococcus lactis
  source 1..174: mol_type = genomic DNA; strain = L. lactis ATCC 11454
  CDS 1..174: note = nisin gene from L. lactis ATCC 11454; protein_id = 2; translation = MSTKDFNLDLVSVSKKDSGASPRITSISLCTPGCKTGALMGCNM KTATCHCSIHVSK
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 2 - protein, 57 aa, Lactococcus lactis
MSTKDFNLDLVSVSKKDSGASPRITSISLCTPGCKTGALMGCNMKTATCHCSIHVSK

SEQ ID NO: 3 - DNA, 13323 bp, Lactococcus lactis
  source 1..13323: mol_type = genomic DNA; strain = L. lactis ATCC 11454
  misc_feature 1..13323: note = nisin gene cluster from L. lactis ATCC 11454
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataaccaaatcaaaggatagtattttgttagttcagacatggatactatcctatttttataagttatttagggttgctaaatagcttataaaaataaagagaggaaaaaacatgataaaaagttcatttaaagctcaaccgtttttagtaagaaatacaattttatctccaaacgataaacggagttttactgaatatactcaagtcattgagactgtaagtaaaaataaagtttttttggaacagttactactagctaatcctaaactctatgatgttatgcagaaatataatgctggtctgttaaagaagaaaagggttaaaaaattatttgaatctatttacaagtattataagagaagttatttacgatcaactccatttggattatttagtgaaacttcaattggtgttttttcgaaaagttcacagtacaagttaatgggaaagactacaaagggtataagattggatactcagtggttgattcgcctagttcataaaatggaagtagatttctcaaaaaagttatcatttactagaaataatgcaaattataagtttggagatcgagtttttcaagtttataccataaatagtagtgagcttgaagaagtaaatattaaatatacgaatgtttatcaaattatttctgaattttgtgagaatgactatcaaaaatatgaagatatttgtgaaactgtaacgctttgctatggagacgaatatagagaactatcggaacaatatcttggcagtctgatagttaatcattatttgatctctaatttacaaaaagatttgttgtcagatttttcttggaacacttttttgactaaagttgaagcaatagatgaagataaaaaatatataattcctctgaaaaaagttcaaaagtttattcaagaatactcagaaatagaaattggtgaaggtattgagaaactgaaagaaatatatcaggaaatgtcacaaattcttgagaatgataattatattcaaattgatttaattagtgatagtgaaataaattttgatgttaaacaaaagcaacaattagaacatttagctgagtttttaggaaatacgacaaaatctgtaagaagaacatatttggatgactataaggataaatttatcgaaaaatatggtgtagatcaagaagtacaaataacagaattatttgattctacatttggcataggagctccatataattataatcatcctcgaaatgacttttatgagtccgaaccgagtactctatactattcagaagaggagagagaaaagtacctcagcatgtatgtagaagccgttaaaaatcataatgtaattaatcttgacgacttagagtctcattatcaaaaaatggacttagaaaagaaaagtgaacttcaagggttagaattatttttgaatttggcaaaggagtatgaaaaagatatttttattttaggggatatcgttggaaataataatttgggaggggcatcaggtagattttctgcactctctccggagttaacaagttatcatagaacgatagtagattctgtcgaaagagaaaatgagaataaagaaattacatcgtgtgaaatagtatttcttccagaaaatatcagacatgctaacgttatgcatacatcaattatgaggaggaaagtacttccattttttacaagtacaagtcacaatgaagttctgttaactaatatctatattggaatagacgaaaaagaaaaattttatgcacgagacatttcaactcaagaggtattgaaattctacattacaagcatgtacaataaaacgttattcagtaatgagctaagatttctttacgaaatttcattagatgacaagtttggtaatttaccttgggaacttatttacagagactttgattatattccacgtttagtatttgacgaaatagtaatatctcctgctaaatggaaaatttggggaagggatgtaaatagtaagatgacaataagagaacttattcaaagcaaagaaattcccaaagagttttatattgtcaatggagataataaagtttatttatcacaggaaaacccattggatatggaaattttagagtcggcgataaagaagagctcaaaaagaaaagattttatagagctacaagaatattttgaagatgaaaatatcataaataaaggagaaaaggggagagttgccgatgttgtagtgccttttattagaacgagagcattaggtaatgaagggagagcatttataagagagaaaagagtttcggttgaacggcgtgaaaaattgccctttaacgagtggctttatctaaagttgtacatttctataaatcgtcaaaatgaatttttactgtcgtatcttccagatattcagaaaatagtagcaaacctgggtggaaatctattcttcctaagatatactgatcctaaaccacatattagattgcgtataaaatgttcagatttatttttagcttacggatctattcttgaaatcttaaaaaggagtcggaaaaataggataatgtcaacttttgatatttctatttatgatcaagaagtagaaagatatggtggatttgatactttagagttatccgaagcaatattttgtgccgattctaaaattattccaaatttgcttacattgataaaagatactaataatgattggaaagtcgatgatgtatcaatcttggtgaattatttatatctgaaatgcttctttgagaatgataacaaaaagattcttaattttttgaatttagttagtcctaaaaaggttaaagaaaatgtcaatgaaaagattgaacattatcttaagcttctgaaagttaataatctaggtgaccaaattttttatgacaagaattttaaagaattaaagcatgccataaaaaatttatttttaaaaatgatagctcaagattttgaacttcagaaagtttattcaattattgacagtatcattcatgtccataataaccgactaattggtattgaacgagataaagagaaattaatttattacacacttcaaaggttgtttgtttcggaagaatacatgaaatgaggactaatagatggatgaagtgaaagaattcacatcaaaacaattttttaatactttacttactcttccaagcaccttgaagttaatttttcagttggaaaaacgttatgcaatttatttaattgtgctaaatgctatcacagcttttgttccgttggctagtctttttatttatcaagatttaataaactctgtgctaggttcagggagacatcttatcaatattattatcatctattttattgttcaagtgataacaacagttctgggacagctggaaagttatgttagtggaaaatttgatatgcgactttcttacagtatcaatatgcgcctcatgaggactacctcatctcttgaattaagtgattatgagcaggctgatatgtataatatcatagaaaaagttactcaagacagcacttacaagccttttcagctatttaatgctatcattgttgtgctttcatcgtttatctcattgttatctagtctattttttattggaacatggaacattggggtagcaattttactccttattgttccagtattatctttggtactttttctcagagtgggacaattagagtttttaatccagtggcagagagcaagttctgaaagagaaacatggtatattgtatatttattgactcatgatttttcatttaaagaaatcaagttaaataatattagcaattacttcattcataaatttggaaaattaaagaaaggatttatcaaccaagatttagctattgctcgtaagaagacatatttcaatatttttcttgatttcattttgaatttgataaatattcttacgatatttgctatgatcctttcggtaagagcaggaaaacttcttataggtaatttggtaagtctcatacaagctatttctaaaatcaatacttattctcaaacaatgattcaaaatatttacatcatttataatactagtttgtttatggaacaactttttgagtttttaaagagagaaagtgtagttcacaaaaaaatagaagatactgaaatatgcaatcaacatataggaactgttaaagtaattaatttatcatatgtttaccctaattcgaatgcctttgcactaaagaatatcaatttatcctttgaaaaaggagaattaactgctattgtaggaaaaaatggttcagggaaaagtacactagtaaagataatttcaggattatatcaaccaactatgggaataatccaatacgacaaaatgagaagtagtttgatgcctgaggagttttatcagaaaaacatatcggtgctgttccaagattttgtgaagtatgagttaacgataagagagaatataggattgagtgatttgtcttctcaatgggaagatgagaaaattattaaagtactagataatttaggactcgattttttgaaaactaataatcaatatgtacttgatacgcagttaggaaattggtttcaagaagggcatcaactttcaggaggtcagtggcaaaaaattgcattagcaaggacattctttaagaaagcttcaatttatattttagatgaaccaagtgctgcactcgatcctgtagctgaaaaagaaatatttgattattttgttgctctttcggaaaataatatttcaattttcatttctcatagtttgaatgctgccagaaaagcaaataaaatcgtggttatgaaagatggacaggtcgaagatgttggaagtcatgatgtccttctgagaagatgtcaatactatcaagaactttattattcagagcaatatgaggataatgatgaataaaaaaaatataaaaagaaatgttgaaaaaattattgctcaatgggatgagagaactagaaaaaataaagaaaacttcgatttcggagagttgactctctctacaggattgcctggtataattttaatgttagcggagttaaaaaataaagataactcaaagatatatcagaaaaagatagacaattatattgaatatattgttagcaaactttcaacatatgggcttttaacaggatcactttattcgggagcagctggcattgcattaagtatcctacatttacgagaagatgacgaaaaatataagaatcttcttgatagcctaaatagatatatcgaatatttcgtcagagaaaaaattgaaggatttaatttggaaaacattactcctcctgattatgacgtgattgaaggtttatctgggatactttcctatctattattaatcaacgacgagcaatatgatgatttgaaaatactcattatcaattttttatcaaatctgactaaagaaaacaaaggactaatatcgctttacatcaaatcggagaatcagatgtctcaatcagaaagtgagatgtatccactaggctgtttgaatatgggattagcacatggacttgctggagtgggctgtatcttagcttatgcccacataaaaggatatagtaatgaagcctcgttgtcagctttgcaaaaaattatttttatttatgaaaagtttgaacttgaaaggaaaaaacagtttctatggaaagatggacttgtagcagatgaattaaaaaaagagaaagtaattagggaagcaagtttcattagagatgcatggtgctatggaggtccaggtattagtctgctatacttatacggaggattagcactggataatgactattttgtagataaagcagaaaaaatattagagtcagctatgcaaaggaaacttggtattgattcatatatgatttgccatggctattctggtttaatagaaatttgttctttatttaagcggctattaaatacaaaaaagtttgattcatacatggaagaatttaatgttaatagtgagcaaattcttgaagaatacggagatgaaagtggcacgggttttcttgaaggaataagtggctgtatactggtattatcgaaatttgaatattcaatcaattttacttattggagacaagcactgttactttttgacgattttttgaaaggagggaagaggaaatgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactagagtgaaaaaaatactaggtttcctttttatcgtttgttcgttgggtttatcagcaactgtgcatggggagacaacaaattcacaacagttactctcaaataatattaatacggaattaattaatcataattctaatgcaattttatcttcaacagagggatcaacgactgattcgattaatctaggggcgcagtcacctgcagtaaaatcgacaacaaggactgaattggatgtaactggtgctgctaaaactttattacagacatcagctgttcaaaaagaaatgaaagtttcgttgcaagaaactcaagttagttctgaattcagtaagagagatagcgttacaaataaagaagcagttccagtatctaaggatgagctacttgagcaaagtgaagtagtcgtttcaacatcatcgattcaaaaaaataaaatcctcgataataagaagaatagagctaacttcgttacttcctctccgcttattaaggaaaaaccatcaaattctaaagatgcatctggtgtaattgataattctgcttctcctctatcttatcgtaaagctaaggaagtggtatctcttagacaacctttaaaaaatcaaaaagtagaggcacaacctctattgataagtaattcttctgaaaagaaagcaagtgtttatacaaattcacatgatttttgggattatcagtgggatatgaaatatgtgacaaataatggagaaagctatgcgctctaccagccctcaaagaaaatttctgttggaattattgattcaggaatcatggaagaacatcctgatttgtcaaatagtttaggaaattattttaaaaatcttgttcctaagggagggtttgataatgaagaacctgatgaaactggaaatccaagtgatattgtcgacaaaatgggacacgggacggaagtcgcaggtcagattacagcaaatggtaatattttaggagtagcaccagggattactgtaaatatatacagagtatttggtgaaaatctttcgaaatcggaatgggtagctagagcaataagaagagctgcggatgatgggaacaaggtcatcaatataagtgctggacagtatcttatgatttcaggatcgtatgatgatggaacaaatgattatcaagagtatcttaattataagtcagcaataaattatgcaacagcaaaaggaagtattgttgtcgcagctcttggtaatgatagtttaaacatacaagataaccaaacaatgataaactttcttaagcgtttcagaagtataaaggttcctggaaaagttgtagatgcaccgagtgtatttgaggatgtaatagccgtaggtggaatagatggttatggtaatatttctgattttagtaatattggagcggatgcaatttatgctcctgctggcacaacggccaattttaaaaaatatgggcaagataaatttgtcagtcagggttattatttgaaagattggctttttacaactactaatactggctggtaccaatatgtttatggcaactcatttgctactcctaaagtatctggggcactggcattagtagttgataaatatggaataaagaatcctaaccaactaaaaaggtttcttctaatgaattctccagaagttaatgggaatagagtattgaatattgttgatttattgaatgggaaaaataaagcttttagcttagatacagataaaggtcaggatgatgctattaaccataaatcgatggagaatcttaaagagtctagggatacaatgaaacaggaacaagataaagaaattcaaagaaatacaaataacaatttttctatcaaaaatgattttcataacatttcaaaagaagtaatttcagttgattataatattaatcaaaaaatggctaataatcgaaattcgagaggtgctgtttctgtacgaagtcaagaaattttacctgttactggagatggagaagattttttaccggctttaggtatagtgtgtatctcaatccttggtatattgaaaagaaagactaaaaattgatagattatatttcttcagaatgaatggtataatgaagtaatgagtactaaacaatcggaggtaaagtggtgtataaaattttaatagttgatgatgatcaggaaattttaaaattaatgaagacagcattagaaatgagaaactatgaagttgcgatgcatcaaaacatttcacttcccttggatattactgattttcagggatttgatttgattttgttagatatcatgatgtcaaatattgaagggacagaaatttgtaaaaggattcgcagagaaatatcaactccaattatctttgttagtgcgaaagatacagaagaggatattataaacggcttaggtattggtggggatgactatattactaagccttttagccttaaacagttggttgcaaaagtggaagcaaatataaagcgagaggaacgcaataaacatgcagttcatgttttttcagagattcgtagagatttaggaccaattacattttatttagaagaaaggcgagtctgtgtcaatggtcaaacaattccactgacttgtcgtgaatacgatattcttgaattactatcacaacgaacttctaaagtttatacgagagaggatatttatgatgacgtatatgatgaatattctaatgcactttttcggtcaatctcggaatatatttatcagattaggagtaagtttgcaccatacgatattaatccgataaaaacggttcggggacttgggtatcagtggcatgggtaaaaaatattcaatgcgtcgacggatatggcaagctgtcattgaaattatcataggtacttgtctacttatcctgttgttactgggcttgactttctttctacgacaaattggacaaatcagtggttcagaaactattcgtttatctttagattcagataatttaactatttctgatatcgaacgtgatatgaaacactacccatatgattatattatttttgacaatgatacaagtaaaattttgggaggacattatgtcaagtcggatgtacctagttttgtagcttcaaaacagtcttcacataatattacagaaggagaaattacttatacttattcaagcaataagcatttttcagttgttttaagacaaaacagtatgcctgaatttacaaatcatacgcttcgttcaatttcttataatcaatttacttaccttttcttttttcttggtgaaataatactcattattttttctgtctatcatctcattagagaattttctaagaattttcaagccgttcaaaagattgcattgaagatgggggaaataactacttttcctgaacaagaggaatcaaaaattattgaatttgatcaggttctgaataacttatattcgaaaagtaaggagttagctttccttattgaagcggagcgtcatgaaaaacatgatttatccttccaggttgctgcactttcacatgatgttaagacacctttaacagtattaaaaggaaatattgaactgctagagatgactgaagtaaatgaacaacaagctgattttattgagtcaatgaaaaatagtttgactgtttttgacaagtattttaacacaatgattagttatacaaaacttttgaatgatgaaaatgattacaaagcgacaatctccctggaggattttttgatagatttatcagttgagttggaagagttgtcaacaacttatcaagtggattatcagctagttaaaaaaacagatttaaccactttttacggaaatacattagctttaagtcgagcacttatcaatatctttgttaatgcctgtcagtatgctaaagagggtgaaaaaatagtcagtttgagtatttatgatgatgaaaaatatctctattttgaaatctggaataatggtcatcctttttctgaacaagcaaaaaaaaatgctggaaaactatttttcacagaagatactggacgtagtgggaaacactatgggattggactatcttttgctcaaggtgtagctttaaaacatcaaggaaacttaattctcagtaatcctcaaaaaggtggggcagaagttatcctaaaaataaaaaagtaatttagtaatctctaaggattactttttttgtttctgaatagattctgaaaattgttttatatactttttttaaacataaaataaagtgaggaaatataatgcaggtaaaaattcaaaatctttctaaaacatataaagaaaagcaggtgctacaagatatcagttttgatattaaatctggaacagtctgtggtttattaggagttaacggtgcaggaaaatcaactttgatgaaaattttgtttggtttaatttctgcagatactggaaaaattttttttgatggacaagaaaagacaaataatcaacttggagccttaatcgaggctccagcaatatatatgaatttatctgctttcgataatcttaaaactaaggctttgctttttggaatttcagataagagaattcatgaaactctagaagtgattggtttggcagaaacaggaaagaaaagagcaggaaaattctctttagggatgaaacaacgtttgggaattggtatggctattcttacagaacctcaatttttaattcttgatgaacctactaatggtttggatcctgatggtattgcggagttgttaaacttaatcttaaaacttaaagctaaaggtgtgacaatcttgatttctagtcatcagttgcacgaaataagtaaagtagctagtcaaattattattttgaacaaaggtaagattcgttataatcatgcgaacaataaagaagacgacattgaacagttattctttaagattgtgcatggaggaatgtgatatgaaaagaataatagcatcagaagcaataaaattaaaaaaatcaggaactcttagattggtattaattatcccttttgtgactctatttatagcatttcttatgggtggaatacagatttttagtgttttttcaatttattggtgggaaactggttttttattccttttgatgagtttgctttttctttatgatataaaatcagaggagcaagctggaaattttcaaaatgtgaaatggaaaaagctgagttggaaaattcatttggccaaaatgttgttgatttggctaagaggtatactagcgagcatagtcttgattattttgctttatttggttgcttttgtgtttcaaggtattgtagtggtggattttatgaaagtaagtgtggcattgattgctatattactagcagcttcttggaatttaccctttatatacttgattttcaagtggattaatacttacgtattgttagctgcgaataccttgatttgtttaattgttgccccttttgttgcacaaactccagtatggttcttgctaccatacacttatcactataaagttacagaaagtttgttaaatatcaaaccatcaggagatttgttaacagggaagataaatttcagtatttgggaagttttattaccatttggactttccatagttgtaacgataggagtttcgtatttacttaaaggagtgatagaacatgataagaagtgaatgtctcaaattaaaaaatagcttagggttttatttagtttttctctttactttattagagcttttaacggttcctatttatttagcttttggaagaagtcatgtttcaatgactgatttatcgctcatgatttttttgttttttccgttactggttacaattttgtctattctaatctttgaacaggagagtctggccaatcgtttccaagaaataaatgtaaataaaaaaagtagcagaatttggttatcaaagctaatagtagtggatttccttttgttctttccatcagcaatgatctggataattacgggagtttcacaggcagtagggcaacaaggaatgatgatcgcaacagctagctggttgatggcaatttttcttaatcattttcatcttttattgacctttataatcaatcgaggagggagcatgattatcgcgattattgaaatattactcattatttttgccagtaataaagttttattagcagcttattggtgtcccattgctttacctgttaattttatgataactgggcggtgtgcttatctgatagctgccgtagggtggattgttttatccacaataattcttgtagcattatctaaaaaaaagattagat

SEQ ID NO: 4 - DNA, 66040 bp, Lactococcus lactis
  source 1..66040: mol_type = genomic DNA; strain = L. lactis ATCC 11454
  misc_feature 1..66040: note = Tn5307 transposon from L. lactis ATCC 11454
ataatggaaatttttgatacacatataaagtgcgttttaacctagttttaaaagatttactgttaataaaaataaaatgaaccaaagaactaaccaaagcaaaactatacagcatttactttatccaaaacctactgtaaaatttatagtaggttttgtaatttaaaatattaaagtaaatttacaaataactcttaagtgagatagtctaaataaatgaattgatgtgcaccccaaaagttagactttttatccaggtatttattggaaaggttataataaactagacacaaagttaagagaaatcgtggaaaggtttattatgggaagaagaaaattcgataaacaatttaaaaattctgcagtaaaactcattcttgaagagggttactctgttaaagaagtcagccaagagcttgaggttcatgccaatagtctttatcgctgggttcaagaagttgaagaatatggagaaagtgcttttccaggcaatgggacagccctagctgatgcccaacataagattaaattgttagagaaagaaaatcgttatcttcaggaggaacttgaacttctaaaaaagttccaggtcttcttgaagcgaagcaagtaaaacgttttgaatttctcttgaaacatcatgggaagataaaaattaagcatgcagtaaaagttcttaaggtttctcgctcaggtttctatgaatacatgcatcgtcgtccttcaaaacaacaagtggagagagaaattctctcagagaagataaaagctgtctttcatgagcataagggacgctatggtgcggttagaattaccaaggtacttcataatactggtattatgaccaacacgaaacgtgttgggaaactgatgcacttgatgggactttatgccaagggaagccgttataaatataaacattacaacagaaaaggagcttcgctttcaagacccaatttaattaatcagatctttaaagcaacagctcctaataaagtatggctgggagacatgacctatatccctaccaaagaaggtaccttatacttagccgtgaatgtcgacgtgttttcacgtaagattgtaggctggtcaatgtcttcacggatgcaagataaactggtgagggattgcttcttacaagcttgtgggaaagaacatcctcagcctggcttgattgtccatactgatcaagggagtcaatatacaagctctcgttatcaatctactcttcgtcaagtcggtgctcaatctagcatgagtcgtaaaggaaatccctatgacaatgcaatgatggagtctttttataagacgctaaagagggagcttattaatgatgctcattttgagacaagagctgaggctactcaagaaatatttaaatacattgagacctattacaatacaaaaaggatgcattcaggtcttgattacaagtctccaaaagactttgaaaaatataattcttaaattctcttaactccgtgtctagtttttcgttgactttccattatgcttggattttttattgtttaattcccttttttgtatacaagctcgtattcttaacaaataattggcatatcgggtttaaaaatactatgtgttttaaagaatctctcatgagtttgacgccaataacttagattaaaatcaccgtcaccttatttttaggcacgttcggcagtaaccttatcaaaggtatctcggtcattaagtttcatgatagtatttactattttgatggtttttgttattatccaatcgttaaaatgacaaaaacaaatagataaatagataaatatttatggggaggacaagtgaacttatcatgattaattgtaaacgattgagttctgaatgtttcaaattatgaggaacaacaggagttggactattctttaaacgcctcgacgataccatcactcttcattagcctaaaattaacaagttaaaatcattagaataatctcttttacaaaaaatatttatttaagttatagttgacgaatatttaataattttattaatatcttgattttctagttcctgaataatatagagataggtttattgagtcttagacatacttgaatgacctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcaaaatgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataaccaaatcaaaggatagtattttgttagttcagacatggatactatcctatttttataagttatttagggttgctaaatagcttataaaaataaagagaggaaaaaacatgataaaaagttcatttaaagctcaaccgtttttagtaagaaatacaattttatctccaaacgataaacggagttttactgaatatactcaagtcattgagactgtaagtaaaaataaagtttttttggaacagttactactagctaatcctaaactctatgatgttatgcagaaatataatgctggtctgttaaagaagaaaagggttaaaaaattatttgaatctatttacaagtattataagagaagttatttacgatcaactccatttggattatttagtgaaacttcaattggtgttttttcgaaaagttcacagtacaagttaatgggaaagactacaaagggtataagattggatactcagtggttgattcgcctagttcataaaatggaagtagatttctcaaaaaagttatcatttactagaaataatgcaaattataagtttggagatcgagtttttcaagtttataccataaatagtagtgagcttgaagaagtaaatattaaatatacgaatgtttatcaaattatttctgaattttgtgagaatgactatcaaaaatatgaagatatttgtgaaactgtaacgctttgctatggagacgaatatagagaactatcggaacaatatcttggcagtctgatagttaatcattatttgatctctaatttacaaaaagatttgttgtcagatttttcttggaacacttttttgactaaagttgaagcaatagatgaagataaaaaatatataattcctctgaaaaaagttcaaaagtttattcaagaatactcagaaatagaaattggtgaaggtattgagaaactgaaagaaatatatcaggaaatgtcacaaattcttgagaatgataattatattcaaattgatttaattagtgatagtgaaataaattttgatgttaaacaaaagcaacaattagaacatttagctgagtttttaggaaatacgacaaaatctgtaagaagaacatatttggatgactataaggataaatttatcgaaaaatatggtgtagatcaagaagtacaaataacagaattatttgattctacatttggcataggagctccatataattataatcatcctcgaaatgacttttatgagtccgaaccgagtactctatactattcagaagaggagagagaaaagtacctcagcatgtatgtagaagccgttaaaaatcataatgtaattaatcttgacgacttagagtctcattatcaaaaaatggacttagaaaagaaaagtgaacttcaagggttagaattatttttgaatttggcaaaggagtatgaaaaagatatttttattttaggggatatcgttggaaataataatttgggaggggcatcaggtagattttctgcactctctccggagttaacaagttatcatagaacgatagtagattctgtcgaaagagaaaatgagaataaagaaattacatcgtgtgaaatagtatttcttccagaaaatatcagacatgctaacgttatgcatacatcaattatgaggaggaaagtacttccattttttacaagtacaagtcacaatgaagttctgttaactaatatctatattggaatagacgaaaaagaaaaattttatgcacgagacatttcaactcaagaggtattgaaattctacattacaagcatgtacaataaaacgttattcagtaatgagctaagatttctttacgaaatttcattagatgacaagtttggtaatttaccttgggaacttatttacagagactttgattatattccacgtttagtatttgacgaaatagtaatatctcctgctaaatggaaaatttggggaagggatgtaaatagtaagatgacaataagagaacttattcaaagcaaagaaattcccaaagagttttatattgtcaatggagataataaagtttatttatcacaggaaaacccattggatatggaaattttagagtcggcgataaagaagagctcaaaaagaaaagattttatagagctacaagaatattttgaagatgaaaatatcataaataaaggagaaaaggggagagttgccgatgttgtagtgccttttattagaacgagagcattaggtaatgaagggagagcatttataagagagaaaagagtttcggttgaacggcgtgaaaaattgccctttaacgagtggctttatctaaagttgtacatttctataaatcgtcaaaatgaatttttactgtcgtatcttccagatattcagaaaatagtagcaaacctgggtggaaatctattcttcctaagatatactgatcctaaaccacatattagattgcgtataaaatgttcagatttatttttagcttacggatctattcttgaaatcttaaaaaggagtcggaaaaataggataatgtcaacttttgatatttctatttatgatcaagaagtagaaagatatggtggatttgatactttagagttatccgaagcaatattttgtgccgattctaaaattattccaaatttgcttacattgataaaagatactaataatgattggaaagtcgatgatgtatcaatcttggtgaattatttatatctgaaatgcttctttcagaatgataacaaaaagattcttaattttttgaatttagttagtactaaaaaggttaaagaaaatgtcaatgaaaagattgaacattatcttaagcttctgaaagttaataatctaggtgaccaaattttttatgacaagaattttaaagaattaaagcatgccataaaaaatttatttttaaaaatgatagctcaagattttgaacttcagaaagtttattcaattattgacagtatcattcatgtccataataaccgactaattggtattgaacgagataaagagaaattaatttattacacacttcaaaggttgtttgtttcggaagaatacatgaaatgaggactaatagatggatgaagtgaaagaattcacatcaaaacaattttttaatactttacttactcttccaagcaccttgaagttaatttttcagttggaaaaacgttatgcaatttatttaattgtgctaaatgctatcacagcttttgttccgttggctagtctttttatttatcaagatttaataaactctgtgctaggttcagggagacatcttatcaatattattatcatctattttattgttcaagtgataacaacagttctgggacagctggaaagttatgttagtggaaaatttgatatgcgactttcttacagtatcaatatgcgcctcatgaggactacctcatctcttgaattaagtgattatgagcaggctgatatgtataatatcatagaaaaagttactcaagacagcacttacaagccttttcagctatttaatgctatcattgttgtgctttcatcgtttatctcattgttatctagtctattttttattggaacatggaacattggggtagcaattttactccttattgttccagtattatctttggtactttttctcagagtgggacaattagagtttttaatccagtggcagagagcaagttctgaaagagaaacatggtatattgtatatttattgactcatgatttttcatttaaagaaatcaagttaaataatattagcaattacttcattcataaatttggaaaattaaagaaaggatttatcaaccaagatttagctattgctcgtaagaagacatatttcaatatttttcttgatttcattttgaatttgataaatattcttacgatatttgctatgatcctttcggtaagagcaggaaaacttcttataggtaatttggtaagtctcatacaagctatttctaaaatcaatacttattctcaaacaatgattcaaaatatttacatcatttataatactagtttgtttatggaacaactttttgagtttttaaagagagaaagtgtagttcacaaaaaaatagaagatactgaaatatgcaatcaacatataggaactgttaaagtaattaatttatcatatgtttaccctaattcgaatgcctttgcactaaagaatatcaatttatcctttgaaaaaggagaattaactgctattgtaggaaaaaatggttcagggaaaagtacactagtaaagataatttcaggattatatcaaccaactatgggaataatccaatacgacaaaatgagaagtagtttgatgcctgaggagttttatcagaaaaacatatcggtgctgttccaagattttgtgaagtatgagttaacgataagagagaatataggattgagtgatttgtcttctcaatgggaagatgagaaaattattaaagtactagataatttaggactcgattttttgaaaactaataatcaatatgtacttgatacgcagttaggaaattggtttcaagaagggcatcaactttcaggaggtcagtggcaaaaaattgcattagcaaggacattctttaagaaagcttcaatttatattttagatgaaccaagtgctgcactcgatcctgtagctgaaaaagaaatatttgattattttgttgctctttcggaaaataatatttcaattttcatttctcatagtttgaatgctgccagaaaagcaaataaaatcgtggttatgaaagatggacaggtcgaagatgttggaagtcatgatgtccttctgagaagatgtcaatactatcaagaactttattattcagagcaatatgaggataatgatgaataaaaaaaatataaaaagaaatgttgaaaaaattattgctcaatgggatgagagaactagaaaaaataaagaaaacttcgatttcggagagttgactctctctacaggattgcctggtataattttaatgttagcggagttaaaaaataaagataactcaaagatatatcagaaaaagatagacaattatattgaatatattgttagcaaactttcaacatatgggcttttaacaggatcactttattcgggagcagctggcattgcattaagtatcctacatttacgagaagatgacgaaaaatataagaatcttcttgatagcctaaatagatatatcgaatatttcgtcagagaaaaaattgaaggatttaatttggaaaacattactcctcctgattatgacgtgattgaaggtttatctgggatactttcctatctattattaatcaacgacgagcaatatgatgatttgaaaatactcattatcaattttttatcaaatctgactaaagaaaacaatggactaatatcgctttacatcaaatcggagaatcagatgtctcaatcagaaagtgagatgtatccactaggctgtttgaatatgggattagcacatggacttgctggagtgggctgtatcttagcttatgcccacataaaaggatatagtaatgaagcctcgttgtcagctttgcaaaaaattatttttatttatgaaaagtttgaacttgaaaggaaaaaacagtttctatggaaagatggacttgtagcagatgaattaaaaaaagagaaagtaattagggaagcaagtttcattagagatgcatggtgctatggaggtccaggtattagtctgctatacttatacggaggattagcactggataatgactattttgtagataaagcagaaaaaatattagagtcagctatgcaaaggaaacttggtattgattcatatatgatttgccatggctattctggtttaatagaaatttgttctttatttaagcggctattaaatacaaaaaagtttgattcatacatggaagaatttaatgttaatagtgagcaaattcttgaagaatacggagatgaaagtggcacgggttttcttgaaggaataagtggctgtatactggtattatcgaaatttgaatattcaatcaattttacttattggagacaagcactgttactttttgacgattttttgaaaggagggaagaggaaatgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactagagtgaaaaaaatactaggtttcctttttatcgtttgttcgttgggtttatcagcaactgtgcatggggagacaacaaattcacaacagttactctcaaataatattaatacggaattaattaatcataattctaatgcaattttatcttcaacagagggatcaacgactgattcgattaatctaggggcgcagtcacctgcagtaaaatcgacaacaaggactgaattggatgtaactggtgctgctaaaactttattacagacatcagctgttcaaaaagaaatgaaagtttcgttgcaagaaactcaagttagttctgaattcagtaagagagatagcgttacaaataaagaagcagttccagtatctaaggatgagctacttgagcaaagtgaagtagtcgtttcaacatcatcgattcaaaaaaataaaatcctcgataataagaagaaaagagctaacttcgttacttcctctccgcttattaaggaaaaaccatcaaattctaaagatgcatctggtgtaattgataattctgcttctcctctatcttatcgtaaagctaaggaagtggtatctcttagacaacctttaaaaaatcaaaaagtagaggcacaacctctattgataagtaattcttctgaaaagaaagcaagtgtttatacaaattcacatgatttttgggattatcagtgggatatgaaatatgtgacaaataatggagaaagctatgcgctctaccagccctcaaagaaaatttctgttggaattattgattcaggaatcatggaagaacatcctgatttgtcaaatagtttaggaaattattttaaaaatcttgttcctaagggagggtttgataatgaagaacctgatgaaactggaaatccaagtgatattgtcgacaaaatgggacacgggacggaagtcgcaggtcagattacagcaaatggtaatattttaggagtagcaccagggattactgtaaatatatacagagtatttggtgaaaatctttcgaaatcggaatgggtagctagagcaataagaagagctgcggatgatgggaacaaggtcatcaatataagtgctggacagtatcttatgatttcaggatcgtatgatgatggaacaaatgattatcaagagtatcttaattataagtcagcaataaattatgcaacagcaaaaggaagtattgttgtcgcagctcttggtaatgatagtttaaacatacaagataaccaaacaatgataaactttcttaagcgtttcagaagtataaaggttcctggaaaagttgtagatgcaccgagtgtatttgaggatgtaatagccgtaggtggaatagatggttatggtaatatttctgattttagtaatattggagcggatgcaatttatgctcctgctggcacaacggccaattttaaaaaatatgggcaagataaatttgtcagtcagggttattatttgaaagattggctttttacaactactaatactggctggtaccaatatgtttatggcaactcatttgctactcctaaagtatctggggcactggcattagtagttgataaatatggaataaagaatcctaaccaactaaaaaggtttcttctaatgaattctccagaagttaatgggaatagagtattgaatattgttgatttattgaatgggaaaaataaagcttttagcttagatacagataaaggtcaggatgatgctattaaccataaatcgatggagaatcttaaagagtctagggatacaatgaaacaggaacaagataaagaaattcaaagaaatacaaataacaatttttctatcaaaaatgattttcataacatttcaaaagaagtaatttcagttgattataatattaatcaaaaaatggctaataatcgaaattcgagaggtgctgtttctgtacgaagtcaagaaattttacctgttactggagatggagaagattttttaccggctttaggtatagtgtgtatctcaatccttggtatattgaaaagaaagactaaaaattgatagattatatttcttcagaatgaatggtataatgaagtaatgagtactaaacaatcggaggtaaagtggtgtataaaattttaatagttgatgatgatcaggaaattttaaaattaatgaagacagcattagaaatgagaaactatgaagttgcgacgcatcaaaacatttcacttcccttggatattactgattttcagggatttgatttgattttgttagatatcatgatgtcaaatattgaagggacagaaatttgtaaaaggattcgcagagaaatatcaactccaattatctttgttagtgcgaaagatacagaagaggatattataaacggcttaggtattggtggggatgactatattactaagccttttagccttaaacagttggttgcaaaagtggaagcaaatataaagcgagaggaacgcaataaacatgcagttcatgttttttcagagattcgtagagatttaggaccaattacattttatttagaagaaaggcgagtctgtgtcaatggtcaaacaattccactgacttgtcgtgaatacgatattcttgaattactatcacaacgaacttctaaagtttatacgagagaggatatttatgatgacgtatatgatgaatattctaatgcactttttcggtcaatctcggagtatatttatcagattaggagtaagtttgcaccatacgatattaatccgataaaaacggttcggggacttgggtatcagtggcatgggtaaaaaatattcaatgcgtcgacggatatggcaagctgtcattgaaattatcataggtacttgtctacttatcctgttgttactgggcttgactttctttctacgacaaattggacaaatcagtggttcagaaactattcgtttatctttagattcagataatttaactatttctgatatcgaacgtgatatgaaacactacccatatgattatattatttttgacaatgatacaagtaaaattttgggaggacattatgtcaagtcggatgtacctagttttgtagcttcaaaacagtcttcacataatattacagaaggagaaattacttatacttattcaagcaataagcatttttcagttgttttaagacaaaacagtatgcctgaatttacaaatcatacgcttcgttcaatttcttataatcaatttacttaccttttcttttttcttggtgaaataatactcattattttttctgtctatcatctcattagagaattttctaagaattttcaagccgttcaaaagattgcattgaagatgggggaaataactacttttcctgaacaagaggaatcaaaaattattgaatttgatcaggttctgaataacttatattcgaaaagtaaggagttagctttccttattgaagcggagcgtcatgaaaaacatgatttatccttccaggttgctgcactttcacatgatgttaagacacctttaacagtattaaaaggaaatattgaactgctagagatgactgaagtaaatgaacaacaagctgattttattgagtcaatgaaaaatagtttgactgtttttgacaagtattttaacacaatgattagttatacaaaacttttgaatgatgaaaatgattacaaagcgacaatctccctggaggattttttgatagatttatcagttgagttggaagagttgtcaacaacttatcaagtggattatcagctagttaaaaaaacagatttaaccactttttacggaaatacattagctttaagtcgagcacttatcaatatctttgttaatgcctgtcagtatgctaaagagggtgaaaaaatagtcagtttgagtatttatgatgatgaaaaatatctctattttgaaatctggaataatggtcatcctttttctgaacaagcaaaaaaaaatgctggaaaactatttttcacagaagatactggacgtagtgggaaacactatgggattggactatcttttgctcaaggtgtagctttaaaacatcaaggaaacttaattctcagtaatcctcaaaaaggtggggcagaagttatcctaaaaataaaaaagtaatttagtaatctctaaggattactttttttgtttctgaatagattctgaaaattgttttatatactttttttaaacataaaataaagtgaggaaatataatgcaggtaaaaattcaaaatctttctaaaacatataaagaaaagcaggtgctacaagatatcagttttgatattaaatctggaacagtctgtggtttattaggagttaacggtgcaggaaaatcaactttgatgaaaattttgtttggtttaatttctgcagatactggaaaaattttttttgatggacaagaaaagacaaataatcaacttggagccttaatcgaggctccagcaatatatatgaatttatctgctttcgataatcttaaaactaaggctttgctttttggaatttcagataagagaattcatgaaactctagaagtgattggtttggcagaaacaggaaagaaaagagcaggaaaattctctttagggatgaaacaacgtttgggaattggtatggctattcttacagaacctcaatttttaattcttgatgaacctactaatggtttggatcctgatggtattgcggagttgttaaacttaatcttaaaacttaaagctaaaggtgtgacaatcttgatttctagtcatcagttgcacgaaataagtaaagtagctagtcaaattattattttgaacaaaggtaagattcgttataatcgtgcgaacaataaagaagacgacattgaacagttattctttaagattgtgcatggaggaatgtgatatgaaaagaataatagcatcagaagcaataaaattaaaaaaatcaggaactcttagattggtattaattatcccttttgtgactctatttatagcatttcttatgggtggaatacagatttttagtgttttttcaatttattggtgggaaactggttttttattccttttgatgagtttgctttttctttatgatataaaatcagaggagcaagctggaaattttcaaaatgtgaaatggaaaaagctgagttggaaaattcatttggccaaaatgttgttgatttggctaagaggtatactagcgagcatagtcttgattattttgctttatttggttgcttttgtgtttcaaggtattgtagtggtggattttatgaaagtaagtgtggcattgattgctatattactagcagcttcttggaatttaccctttatatacttgattttcaagtggattaatacttacgtattgttagctgcgaataccttgatttgtttaattgttgccccttttgttgcacaaactccagtatggttcttgctaccatacacttatcactataaagttacagaaagtttgttaaatatcaaaccatcaggagatttgttaacagggaagataaatttcagtatttgggaagttttattaccatttggactttccatagttgtaacgataggagtttcgtatttacttaaaggagtgatagaacatgataagaagtgaatgtctcaaattaaaaaatagcttagggttttatttagtttttctctttactttattagagcttttaacggttcctatttatttagcttttggaagaagtcatgtttcaatgactgatttatcgctcatgatttttttgttttttccgttactggttacaattttgtctattctaatctttgaacaggagagtctggccaatcgtttccaagaaataaatgtaaataaaaaaagtagcagaatttggttatcaaagctaatagtagtggatttccttttgttctttccatcagcaatgatctggataattacgggagtttcacaggcagtagggcaacaaggaatgatgatcgcaacagctagctggttgatggcaatttttcttaatcattttcatcttttattgacctttataatcaatcgaggagggagcatgattatcgcgattattgaaatattactcattatttttgccagtaataaagttttattagcagcttattggtgtcccattgctttacctgttaattttatgataactgggcggtgtgcttatctgatagctgccgtagggtggattgttttatccacaataattcttgtagcattatctaaaaaaaagattagataaagtattttttcttatggtaattcgacctaatatgtttttgctatttatctcttatttctgtctatagtaatttattcaaagtacctttagactcataagtttgaataaaatttccatcaatatgtgacagttcttactctaagaataatgtttccgtcgttagttttcttttctcagtaattttttataaagtggtttcctatcgacttacactttttcttttggagttattggtgtcaagctcatgagagattctttaaagcatataacatttttaaactgatatgccagttatttgtgaagaatatgagtttctatataaaagagaaaattaaacaatgaaaaatccaagtataaatacttggatttttcattatttttgatagacatataaagtgcattttaacctagaattaaaagatgtatcgttgataataataaaatgaaccaaagcaaaactgacgttaagtcaatttattagagtcaaaaacgtataattttaatgtatttattttaagtgatttctttttcaaattgattaggcgtaagataccctaaactttgatggattcgttttgaattataaaaggcttcaatataccagaaaatactctgataggcttcttcaaagttcttatatttaaattgatagacccactctctttttaaatgtccatgccaagattcaagactggcattatgataaggatagcccttacgactgaaggagtgagtcattccagagttttttattgtctcttcatactcatgactcgtatactgacttccttggtcagaatgaagcatcacagcttctggataattttgtgattccattgccttattcaaagtcctttgcactaattctagagtcattcgctttcctaaatcccaagcaatgacttttttagtataacgatccataatggttgagagataaacccagccttgttgagtaggaatataagtaatatcagttgcccaaaccttatttatctttttaggctcattttgttaataccatcacttacggagtgtccgggtttaaattttttaaggacgacggacttgagctgaagttgtttcataattttttgtacgagctttatccctactttttccccttgattgagtaagaggtgatgaatcttaggtgccccatagattccccgattcgccttgaaaagttggtcaatcttcagtgccaagtgttgtcttcttaattgagttttggatgggcgtcgattgattctctcataataacttgcttcaggaacaccaagcagttggcaactgagtctgacattgagtgctaaagttttaatcgtttgagatatatccgcagcactcacatctttttctcggcgaatatggtcaatacttttttaagatgtctcgttcttccttaactttagccagctgtctttttaattctagaaaatcatctttagagactgagctttcattagacttagaatagaggtcgatccatttataaatcgttgcaggagccacgccgtattctttagaaagctgtgtgacggattgcccagaatgatagaagtcgagaagggtgtctttaaattcttgtgagtagcgtttttgcatgtttttatcctttgtctaaattatacaatagtgactctaagaattaaggatagcatcacagtgtctttttgaagaagtaaaatatatttttaatctataccataatgtagctgaacaggaagttgatatgtaatgtcaatgttatctccatcaatttttcgtatgattaaatcaactaagagttccgctaaatcatttattggttgctttatcgttgttaggttaggatagtagttttctataaattcagttccatcaaaacctaccacattaagtgatttttttaaatttgaggcgagatcttttactaatatagctgttaaatcatctgtacaaaatacaccatcaaaatcgttgttttctagtatttttttgatctcaacttttttgcgaagaagagtccattgagaaggaatttttattatatgagtctttaattgatttctttctagctcaagcagatatccatcacgccttaaatatgtaggagaattggtattatcgttcccagtaaatatggcgatttttttacttccagagttaatcaaagctttagtagctaggattcctccctcaaaattatcagagctaacaacagtcgtttcaggggttaataaacggtcaaaagcaacaataggtgcttcaattgctatataatcatgtgcttttaaattatgactcccataaataattccatcaacttggttagaaagtaataaggcaagatagtctcgctctttttgttcatcatgttctgttgttgctaacatagctttgtaacctcgtatgaaaagagcttgctcaattttttcaatcaattctgcataaaaaatattttttatagttggaaatactagcccaattaattttaaagactttccttgcaaagaacgtgcggctgcgtttggaatataatgcaagtcttgcattgctttttctacttttgatattgttgcgtcactcaaatatcctttacgatttataacacgcgaaactgtcgttacactaaccccagctttatttgcaacatcttcaagtttaatcacttttttagctcccataattttatttttatcggattttgacttctaatcgaatactgattcttatttggaaatacacgacctgataatactttttcaccatcattaataaaaatttcaaaaattgattgatcaataaatacattcattttttctattttaacatgtgccaaacgtttttcataatttcttataagtgtaatagtattattttctttatctatctcaactttaagtgcactatcccctttttcatttgtcattaaagatagaagtgtagaggtttgctgacgaaaatcgacctcaagttcataagagttagatgtaataatgttattatctgctagtaagagatcttgttccatttgtcttaattctttcattttttcaactggatattgatacaatttgttgtcttttattgaaagcttcttaaccatggacaagacaccttgaacattgtatttatcagtggggtatgatgtctcaggtaaacctagccatgaaatggcataagcagagccatcaggtgcattaaatgactgcgtagcataacagtcaaaaccttcatccaaattaattagttgtcctgcattttttagctgattttttgagccagtagtaaagtcatccgcaataacatacacattaggatatatattatcatatttaacaatcgatttatctagtccttgtggacaaaaaactaatactgatctaccattgataaatataaggttcgggcattcaatcatataacccattttttcttttgaaaaatctaagtttcctaaatctttccaatcagttaaattattttctattgctttataaagtttgattataccatttttttgagagctttgagcaccaattaagcaataaatttgtccttgaaatgaaaaaatttggggatctcgaaaatggtcggttgtttgagaaaaatcaggataaattagtggttcagtgaatttgactaattgattatttttgtcaatttttgctcccagttgatatggagttctaacccaatcttcgcctctgtgattacccgtatagattaaaaataagaagttttcaaaagccaaagcggacccagaataaactccagcattatcatattttgtatccggataaaggactagccctgttttctcaaagtggacgagatcatctgagactagatgtacccatgattttaaaccatgtactggcccaaaaggaaaatgttggtagaacaaatgccatttttcattgaaataagagaatccatttggatcattaagaagtccagtttctggttcaatatgaaaatttgatttccaaggagattttagtgccagtttgcgtaaactctctaaatcactttcagaataactatcgtaggtacgatatcgttgtttggtagaccatttcattttttttcctcatttactaaattatatatatttatgataaacgttttttataaaaaaatcaatataaataagtataaaaaaataaaaaataaaaaaagtgtggcaaacgcttgacatatatcaaaaaaatgataaaataacttctgtaagcgaaatcattttatttgaggagaaaattatgaatcataagcaggtagctgaacgcattttaaatgcagttgggcgtgataacattcaaggagctgcacattgtgcgacacgactacgtttagtcctaaaagatactggcgttattgatcaagaggctctagataatgacccagatcttaaggggacttttgaagcagctggtcaatatcaaattattgtagggccaggtgatgttaacactgtatacgaagaatttattaaacttacaggaataagtgaggcatcaactgcagatctcaaagagattgcaggaagtcaaaaaaaacaaaatccagtaatggcacttgtaaagctgctttctgatatttttgttcctttgataccagctcttgtagctggtggtctgttaatggctcttaataatgcgctaacagctgaacatcttttcgcgacgaaatcacttgttgaaatgtttccgatgtggaaaggatttgctgatattgttaatactatgagtgccgcaccatttacctttatgccaattttaataggttattctgccacaaaaagatttggcggaaatccatatttaggtgctgttgtaggtatgattatggtgatgcctggacttattaatggatataatgttgctgaagcaatctctaatcatacaatgacttattgggatatttttggttttaaagttgcccaggctggatatcaaggacaagttcttcctgtaattggagtggcttttatccttgctaaacttgagagattctttcataaataccttaacgatgctatagacttcacatttactccgttactttcagtcatcataactggatttctcacattcactattgtgggaccagcacttcgttttgtatcaaatggcttgacagatggtttggttggactttataacacattaggggcactaggaatgcttgtgtttggaggcttctactcggcaatagtagtaactggattacatcagagttttccggccattgaaacaatgttaatcacaaactatcaacacagtggtattggtggagactttatttttccagttgcggcctgcgcaaatatggctcaagctggtgcaacttttgcaattttatttgttactaaaaacattaagacaaaagctcttgcagctccagctggtgtatctgctattctaggtattacagaacctgcgttatttgggattaatctaaaactaaaatatccgttctttattgctcttggggcttcagcaattggttcattatttatgggattattccatgtccttgcggttagtctgggatccgcaggattaattggctttatctctattaaagctgggtataacttacaatttatgatttcgatatttattagctttcttattgcttttgttgttacctcaatctatggtcggcggatggaagctaaatctattacgaaagaaaaaaataaacagaatgcaacaactcaataccaacctgagaaagttattatcgatccagttaaaagtgacgaactccttgctccgataaatggatttgtgattcctctgtctgatgtaagtgaccctgttttctcaaaagaaattatgggaaaaggtattgcaatcaagcctaaatctggagaacttttctcaccagccgacggagaaattattattgcttatgaaacaggtcacgcttatggtataaaaacaaaaaatggagctgaagttcttttacatattggaatagatactgtttcaatgaatggtaatggattcatacaaaatgttaaagttggccagaaagtaaaagcaggggatttactaggatcttttgataaagaagaaatcaagaagagtggattcgatgatactgtaattattgttattacaaattcagcaagttacaatgagattttgccattgagtgaaaatgttgatatcaaagttggagaaaaaattctattattgaactagagggagcgaactatgtccgtatactatggatcaattgaggctggtggtacaaaatttgtacttgctattgcagatgaacattttaatattataaaaaaatgcaaatttgctactactacgccacaagaaactataagtaagacgattaaatactttaaagaaaaccgagtttctgccattggtttaggatcatttgggcctattgatctaaacctttcaagtaaaacgtatggttacattacttcgacgccaaaagttggttggaaaaatataaatttagttggccaattaaaagaagcacttgatattccaatatattttactactgatgtaaacgcctcagcctatggtgaaatgaaaaatacaggaataaaaaatcttgtttatttaacaattggtacaggtattggagggggagcaattcaaaatggatattttattggtggcattgggcattcggaaatggggcaccaaagaataaatcgtcatagagatgatcttacttttgaaggaatctgtcccttccatggtgattgtcttgaaggagttgcggcggggccgagtcttgaagctagaacaggaatacttggggaaaaaatttcttcagatgatcctatctgggatatcttaagctattatattgctcaagcggctattaatgcaacattgacattagctcccgagtgtataattttgggaggtggtgtaatggaaaaaccaaatatgatatcacttatacaaaaacaattcatttctatgcttaataattatatagatctgccttgttctgtagaaaagtatattcgcttaccaactgtcaaagaaaatggttcagcaacgttaggaaatttctatttagcatattctttatttacaaaagaataataatataaaatctatatatttctagcatgtagttcaacaaaatataatggtaattgtaaatattaaggcaatattacaattataaaaaggtttcatttctttgttatcttttaactttttgcaacataatcgaaagttcttttagtgatactaaatacctaataatttaagtatgagaaaagtaaatttctaggtcttactttggaaggcagtatttcacaaattatgtattatcttatttagaataagaaaagttgaaatgcccaaaattatttcaaatatgggaatattattggatgcctgtgtagcaatgcattatagaaactaaaataaaatttagtgggcaaatagtgggtaaaatttaaaatatataaatcaactagacattattgaaagcaatagaaaacttataaaataaagagccactaaactttagtgactttttattttatgtgttttaatattaagcaaacgcgttgagcgtttatttcaaatatgggaatattcatgactgcctttgtaacagtgtattataaaaattaaaataaaatttaccgaataaatcgatactagaaaaactcttaataattttatatttacttaaaaaacaagtgtaaaattataaaaatacaaaatagttataaaaaaagatatatattgtaaggagaagaggggattatttaggtcgctctcaaagttaaattcaggttctataagcctttataaacctgtttcatagaaaacctcttaaattgaaaaataaaatgggacaaatttgggacaattatggtataaaatcatagctacgatttaatctcattgaatttttgagtttataaagttaaggaactaagcatattattttggaagatttgaacaaaaaataaattaaagagtcattaagaattgaattaatgactttttattatgttaagtttgtttgatgcttttaagctatatgtttttctctttataaactaataatcagtattgagcttaacaaggtgctatacttaattttatgagatcacagtctctgttgtggaatagaaaccaaatatggtttatattttttgattgatattgtaatttagaattaaagtgatatacttaccttagggtgtggttttgtgaaagcaccacacctccatcattagctactcaaatggggtagctttttttaaaatatgtaattataaacaaaaaaataagttgataataaggggaagggggcatctaacgaataaaaataaagagaaaaaggcgattataagcttactgttttttggatttgtagtaagtatattaattatatacctagtaataactttactgatgtgagaaaaaatgctgttcgggtaaggtaacacatttgtaaaataatttacgaaccctaatgttaaattgactttacctgatattagtgttatacttgcgaagcgggatggagcagttaggtagctcgtcgggctcataacccgaaggtcataggttcaaatcctattcccgcaatattttaagggctttagggagtccttatttttcttataaaaaactcaccctaaagtgaaattttttcgttaaataggcggagcctagaatggaagtgaaaaatgaaaacaggagataaaattacattatctaatggggaacaagcaattgtcgtttctggagatattaatctctataagtatgcactaattgttgaattagaaaaccatgatgtcagggtggttgatagagaaaccttgacacttgctaaagcaaatcctcatgaaaatctggggaatcataagaagatcaataagttttaaatagaccaatttgactttagtaagaataggtgttactattaagaggtaggttttgtctcgagtttaaatgttttaggaatgcactttttcgcaaaataggatttacagatgatgcgaaaggatgtattatatgaataaaggaacaataaattggtttaacgctgacaaaggctatggttttattatggcagatgatatgcaagatgtgtttgcttatctcttatctattcagggaaatgattttaaaaaatacgatgaaggtcaaaaggttacttttgatatcaaaatgacgtctcgtggtcgttatgcttcaaacgtacataaaagataaatgagttaaagcatggggtgtcctttgcttttttatttattgctattgcaaaaacgaattaaaatgttatactgagtgagggaaagattagttgctttccctacagttcataaaatccatgtcactacgatatggattttattaatcagagagtagcttgacattgagtataattagtgttactatgaagaagtagattttgtcatgagtaaaatgttttatagattcacttttcgcaaaataagatttacaattttgttgaaaggaaataaatatattatggcaaatggaacagtaaaatggtttaacgctactaaaggatttggttttattacctctgaagatggtcaagatttgttcgctcacttctcatcaatccaatctgatggattcaaatcacttgacgaaggtcaaaaagttgaatttgatgttgaagaaggtcaacgtggacctcaagcagttaatatcacaaaagcataattgtatctgaaaatcgtgcaaaaactggagtcatgacttcagttttttttatattcagttgcaattatgagagagtatgatatacttagccagcaagaggaaactcacacttcccattcataaattaggggcactttaatatagagtggtttttttaatgtttattaattaaaattaatatttaaaagatttatataaaaaaccatctatcacagcacataatgataagcagtttttggtgcatgttcatagattaagaagttaatatttaaaacatacaaattaataataccatatagatagtcttaaggtcaaaaatagagagatgtgtttaatggagtaccttaaatataagtgccagcgaatgtagtgacccccaaaatatagacttttcaaatctatatttcggaggtcttttcttatggtcaaatattccatagaattaaaacaacgtgttattcaagattatttatctggcaaaggaggctctacctatcttgctaaaatgcacaacataggttcatctagtcaagttagacgctggattcgcaattatcgagcagagggacttcctacagctcactccaaagtcaataaaaattattctatggaattgaaagaaaatgcggtacaatgttatttaacaactgatttaacttatgaagctgtggcaagaaaattcgaaattactaattttaccttacttgcaagctgggttaatcatttcaaactctatggagaagtcccaataagcaaaaagagaggacgacgtaaaaaactagaaagcattgcatcgtctatgactcagaactcaaatgattctcaacgaattaaagaacttgaacaagaattacgttatgcgcaaattgaggtagcttatttaaaaggacttcggagattggagaaaaatgctctaaataccagccccgataactcaactactggaataacgaataaatcaataaattaacaagggtgtattgctacagacaatttaaaaatgactttgagaatatgaatctcagagtcatttttaatgtaaaatattttatgtttactttaaatcattataattataaatgttataattatgtagagggccaattatgaaaaaaattacttgggaacaatttgaggcggtaaatgaaaataaaacttcatcatttgaaaatatgtgtcgtttactatttaataggcaattttttgataacaaaaaaaatttgatatcaaagccgaatcatccaggagttgaggtctatccagtattcgaagaaaagtcaggaaaatggataagtttccaatcaaagtttttttcaaataatactgattatgctcaaatcaagaaatcagttgaaaaaacaattaaacattactctgatagtttagacgttttttatttgtactgcaataaagatctaaattctgattctcggagttataaaaaaattgaagatttgctaaattcagctggaattgaaattcagttaattactaattatgctatcctcgatcaagtaattggagactcgagaattgctgagtttttttttaatcaccattctctaaacgataattggtttcaagaaaaattggagcaaagtcttgactcaataggaactcgatacaacaatgaatttagtatatcaactgaaaccgagaaagaatttagactttttctaaaaaattcggaaacattgaaaattattgaagaaaaaaaagacgaaacaattaacgaacttaggaatttaagacaacacctttcaggtgagaacgacttatttgctaaatctataatcgtcaaaatacagtctttaaaaatcacggaaataaacaatattgagaactgcctctcttgggaaaatacaatattggaagaattttcgtgtgagtttaagagactagaagctgaaatagaagcaaactataaaaagcaagataaggagaatcagaataaagagaatggagatagactttaccaggctaatttgcatggtttattaaatatttcttcgtatttaaaatttagtgattttgagactaatctgataaaaagtaagatgcttgttataaatgggcaggctggaactgggaaaagtcatcttttatctgaaaatgccaataaaattgtcaataatggtgggcgtgccattttattaccagggcatgtgtttgtatcccaagaacctattaaaaatcaaatagtgtcttatttaggtctcgaaattaattttcgcgaacttttggatatccttgaaacggtaggtgaatttactaaggcaaatacatatttatttattgatgcggtgaatgagagcaataataaagagatctggaaaaatggtttaccaatattacttcgcgaaattgaaaaattgaatttcgtaaaagtcgcaatttcacttcgaagcggatatgaagatacggtttttgatgaaaatatccgtaggaaaattactgactacgagattccagagatcacacataatggattttggaataatagtattgaggcagtaaaagaatttcttaattatcatcaaattccattttctccttcatattcttttcaatctgaaatggcaaatcctttgttcttaactatgttctgcaaagtttatgatggaaatgattttgatttatatcaattacttgaaaaatttcttgaaaaagcagatagagatgcacaaagggcgataggaatacaagattttttaccgatattaaatgacttagttaaggaaattgcagattttcagcttgaaaatgggctttatgctgtaggtaggaatgatattctaaaaatggagttttgggatcttaatggtctctcaaataaaaaacttcaatatttatcatcacttggtaaatctggagtatttgtgactttcatgcgtgaaggggaagaaaagtatagatttgggtacaatctattagaagactttatatgtgccaaagtgatcattaatcgtttttcaagcgctcaagagtgtagagaatatttacgaaacgatttactaaaaattggcaataaagagagaaaggtatgggagaatgctgatgttttcatcgtagccacaaatttatttttcgagaaatttggggaagagtgtattgatattgctaatgatatcacggatgaatacgaccgaaatcatctacttgatgaatatatcaaatcattttcttggcgaccgtcacaaaatattgatcttaaaaattttcgcgaatttataaatgaaaataagattgctcgggacactattttcaatgttttgattgaaaatagtgtcaagtccaataacccattgaatgctgagtttcttcatgaaatactttttaacaaattattgaatgagcgtgattatatgtggaccacctacattaacaattttgcttatgaagacgatagattatttcaattgatcagtttatttgataagggtaaaaagctagagaattctagcgaaaatacttggttattacttgtattattttcttggctgctgacatcgtcgaatagattgttgcgagataaagcatctaaggcgatgattgagttgttgaaagatgatttttcgttaattttaccacttctgaaaaaatttgagaatgtgaatgattcctatgttattcagcgactatacggcgttgtatttggggcttgtacgaaaaatttaaacatgacagaaccagagtttaaagaattggcggaatatgtttacattgcaatatttgataaagatttagtttatccagatattctattgagggattattcaaagctaatcattgagtatttcttgtttaaatttccagaatctaaaacgattattaacgagataacattcagaccaccctataattctaaaccaataccaatcgcgaaaaaagcgaaagataagtatgagggcggactaaatattatcgctcattccatagcaccaaaaggtgtggagagaatgtatggagatttcggtcgatatgtatttgattcagctttgcatcatttttccgatgttgattcagtcaatatttataactattcgatggattttattgaaaatgaacttggttataaaaataaattatttgccgaacatgatagtcttcgaaatcgcaactcttatgacaggcataatacgggaaagattgagagaatcggtaaaaaatatcaatggattacgatgtataacattttggctagggtttccgaccattataaactttctgatggatatggatggaataatagcgaagaaccagaagaatttgacggtgcgtggaatccttatgtacgcgattttgacccaacgctaaatcgtaattttcttgatgatccgagttgtccagttttcgtgaaaaaagagctaaaagggttagattttatatcaaaagatgcttcaaaggaagtcgtcaagaaatgggttagcaatgaagacgatgttttctttactgcaggaccagatcttctgcttttggatgagtcgcaaactgaatgggtggctttagatgattatatgagtataaaaaatactgatgatgaattagaccttattagtattggagatagatcgggtaaacagaaaaaatggcttatagcagacgggtattttatcaagaaaagtgaattttcggacttacaaaatgatttgtcaagcaaaaatcttagaaaaatatcattcactgaaagttcaagaaacatctatcaactatttaaccgtgaatatggttggtcatcggggtataagaggtctgtagcagattgttcttggtcagattatgaggttgagacaggaaaatttgacactattgattatccgagttttaatatctcaatcattgatgatattgatggtgatgaacttgaaactcttgaatatacggacgaaaagactgaaaaatataaagtgccaattaaaaaatctctcgcgagagttatgtcggcaacagatagttttttatggaaagaacaatatgacgcttcgcaagaggaaacgatagcatttgatattccatgtagtcttcttataagtgaactggaattagtgcaacgagaatatgatggctactattatgataaaaacggagaactagtggcactttatattaaagatagtgaagaatttgatagttcgcacagatttttaatccgaaaggggcatttaaataaatttctagctcaaaatgaattgactttattctggatttgtcggggtgagaaagattttatggttcaagaaataagagagcaagaatggagcgaatggggtggtttactatatctttccaatgataaaattgtaggtgaaatagtaaaatttgatagaatatagtcaatacgcttataatgaatcagtaacggtgtattgctacatagttttataaaaaacaagcttcatcgcaatgatgaagcttgtttttaaattgcgtataataatatctaacttaatagttatagtaatgtgcagtatgttttaatatcaggaatatttgtaatcgtaattggaaaaacatgcaatcatagcagttattatgatacaatttaaaaaggcatatttacaagagactgaaaggaatgagagtatgacttttttcactcaaaaggctatggaagttgctaataaagaactgggtaaaaaacaattagctggaattagccaatatacagtgaataagctgaattgaggtgaggcagtgacgataaaaactttatcaaagatttgtaatgctttgcgtagtaggatagaagatatttttgaagtataaagttttaacgaggaagctttaaattaagtggaggaacaagtgtgacaacgtcagaagaaattaaaaaaagattgtgggatggtgccaccgagctacgtggttcaatggatgctagccgatataaggactacatgcttggactaatgttctataagtttttaagtgataaaaccttagagacatttagaaatactgcgggcttaggcagaatatcagaatcagatttagttgaagagtacacccagaatcgtgaagatttaggcgaagaactcgataagatgattcaacaggtcttgggttattttgtagcacctgaatatctttatcagaaatggataaaggatatcaatacgggtgattttgaagtccaaaaagtaaccgatagcttaaacagttttgagaagaccatcgcagtaacaggcgagtccgctgattttaaagggcttttttcgagttctactttggatttgaccgatacagctttaggaagcaatctaaacgaacgtagcaaaaacatcaaagccttaatcaatttgtttgctgacctcgatatggttgctttacaaaagagtgatgtgcttggtgatgcttatgaatatctgattggccagtttgccatggaatcgggcaagaaggcaggggaattctacacgccaagacaggtttcagaagttatggcacaaattgtggcaaagacttcaaatatccaatctatctatgacccaactgtaggttctggttcgcttttattaactgttggaaaacacctgagcaaagaagtgcaaaaagatttatcttactatggtcaagaaaaaaatacagccacttataacctgacacgtatgaacttgctcttacatggggtacgtccagaaaagatgacggtaagaaatgcggatactctatctcatgactggcccgaagatccaagtcgaccaaatgtgggcgtacagtttgatgcggttgttatgaacccaccatactcactcaaggactggaataaagctggcttaaaaatcagcgaccctcgatttgagattgcagggacattaccgcctgattctaagggagactttgccttccttttgcatggccttttccacttgggaacaaatggtacaatggcgattgtcttgcctcatggcgtgctctttagaggtggttcagaaggtgatattcgtcaaagacttttagataaaaatcaaattgataccatcattggcctgcccagcggaatgtttaccaatacggggattccagtgatcgtgatgattttgaagaaaaatcgtccagttggagaaccagttttagtcattgatgcatctcgtagttttatcaaagttgggaagcaaaatgttctacaagaaaaagatattgccaaaatcgtagatacttattcatcacgtcgtgagattgaaggctacagctacctagcgactcataaggaaattattgctaacgagtggaatatgaacattcccaggtatgtggaagctgacaatgatgagattgctcaagatgtggatgcccatttatatggtggaattcctaagtctaatattgatgagctgcttgtattaaataagagcgttcctgaagtgattcgttcatcatttgaggtactacgcgatggctacctggtgctaaataagagcattgaacaattgacggaagaagtcaacaatgctccacaagttttggcaaagaatgaggagttgagaaagattgctgaaagtttcgttgctaaatattgggatattctccgtcaggtcaatcttgatagtgatttggctagtctcatgtcagaaatgctttctgaaataaaggaagaggtttctagtattgaatttgttgacgcatatagtgcataccaaatcgtggctgaaatctggaaggacaatcttactaaagatagtgaattgattgcggcaagtgatttttataccgttggtcgcacgcgtgaaccaaatatggtgacaaaaggatcaggcaagaacaaacgccaagagcaagatggctggattgggacgcttgttccaaatggcttgatagcaaaacgtctttttgcttcagaacaagaagaaatcgaagagttgaaaaataaagctcaagaaattgacagcgaattgtctgagcttgtagaggctgccaaagttgaggattctgaagaatatgaggccctttacgaatctatcaagaaaaatgaggatgacgagccacaggatacttttgaagctaaaacgatcaaggctgaactgaaaagatcagaaaaaggaacatctgaatttgagtggctaaaacgtgttgaaaaacttctggcggataaaagttcaacgaataagtccgtcaaagaaaaagaaaaacaactcaaagaagcggttgaatctaagattgaaaatctgacaaatgaagagattgatatgctggtttttgagaagtggtttgcgggtacagtagatgctcttgtagggctagttgaaaagcctttgcgcgcggagctttcaacgattgcacttcttgaaaagcgttatgctcaaacattcaatgaaattgatgcacaagtttcagagctagaaaaagcttttgaagcacttgcgagtgaattggtggtgaatcaatgagcaaaaataaaaaattagtaccgaaaagacgtttcaaggcgtttcaaaatgctgacgcttgggaacagcgtgagttgggggatctagctgaaattgttagaggagcatcaccaagaccaattcaaaatccaaaatggttcaaccaaaattctgaaatcggatggttacggatttcagatgttactgaacaaaatggccgaatacattttcttgaacagagaatatcagaagctggacaaggaaaaactcgtgttttgcattcaagtcacttattacttagtattgctgcaacagttgggaaaccagttattaactatgttccaaccggcgtacacgatggatttttaattttcctaaatccaaaatttgacctagaatttatgtttcagtggttagagatgtttagacctcaatggcaaaaatatggacaaccaggaagtcaagtaaatctaaattctgatttggtgaaaaatcaaaaaatttttataccatcacttggggaacaaaaagagattagttcattttttactaacctcgaccaaaccatcgcttttcaacagcgaaagtttgagaagatgaagtcgatgaaattggcttatttgtcagaaatgtttcctgctgaaggtgagcgtaagccaaaacgccgctttccaggctttactgatgattgggaacagcgtgagctcttaagtacaataaaatccattgtagattttagaggacgaactcctaaaaaacttggcatggattggtcagattcaggttatttggcgttatcagcgttgaatgttaagaatggctacattgatttcaatgaagatgtccattatgggaatcaagagctttatgataaatggatgtctggtaaagaactgtataaaggacaagtactttttacaacagaggctccaatggggaatgtggtacaagtacctgatgataaaggatacattttaagtcaacggacgatagcctttaatattaacaaggatttgttgactgatagttttctttatgtcttactgggaagccttaaggtgtttaaggatttgtcagcattatctagtggtggaacagcaaagggtgtcagtcagaagtcgttagaacagttaaaagtttgcattccaaaagatatagatgaacagtctaaaataagcgagttttttatcaacctcgaccaaactatcgcttttcaacagcaaaagcttgaaaagctgcaaaatatcaaaaaagcatacctcaatgagatgtttatttaagaaaggagcgtaagctgtgagtaatgcacctaaagatgcgagtgaaaaagcctatcaagacaactttgtagctgaactggcaaagtttaaatggcaaacgcctgactttttgaatgggaacaagcaaaaagttaccgttcaaagcctcattgataactggcgaagtgagctcaatcgcttgaataatgaccagcttgaggggatttctttgacagatggtgagtttgctcaagtgctgtcaaaagttggacagattagcaactcctacgaggcagctaaagtgctggcaatggaggaaggtaagggtaaaattgacggcatctatcgagatagccatccgcaagtcacccgtaagcaaatcacgctaaccatcttcaaaaaagctgaggtacgtggaggagaatcaagctatcaggtggcacgtgaagtggagacgtctaatgggaatcgctttgacatcgtgcttttaatcaatggcttgccactgataaacattgaacaaaagcggacagacaagtcgattgatgaagcttttggacaattccagcgttattatcgtgatggcgaatataccaataatttcatggccttttcgcagatgatggtgataacaactgagattgaaacgcgctactttgccacgccaaaaacaattaatgacttcaatcctgcctttgtctttcactggtcagacagaaagaataagcgcatcaatgactggaaaaaggtggtagaacactttttgatgattccaatggcgcaccaaatggtcggtgattacttggtcattgatgaagccaaggaagaagaaaatcgtaggcacatgctcatgcgtccttatcaagtttatgctcttcaatccatagaaggggcagcctttggttgggataatgacgaaaagattccgcatggcggctttgtttggcataccactggttcggggaaaacaatcaccagctttaagacggctctatttctatctacaagagcaggttttgataaggtcatttttcttgttgacagaagagagctagacagccgtactggggaaaatttcaaagcatacgctgcctacgaacctgtagatgttgatgatactcgctccacttatcagctgaaaaagaagctagggacagtcaaaaatggtatcgttgtaacgactacttttaaactaaacaatttgattgaagatttgaaaaagaatgaagactacagtctttcagagaaacgctttgtttttatcattgatgaagcccaccgcaccaccatggggcagatgatgggaactatcaaagactatttcagaaaaaatggcttgttctatggttttacgggtacacccctttttgatgaaaataaggttaaaggcaaaataaataaaaagagtgaactcatcaatacgactgaaaaactctttgggcctgaacttcatcaatatacgatagaccaagcgatttctgacgggaacgtccttggtttccacgtggattacatcaatacgggtgaattcaagagttatgaagatttgcgtgaacaactgcaagataaggctttaatagagaatccagatgtcgataagaaagaacttgaacgtaaatatgcagtgatgtccgagctggaagtggaaaaagaagcagctaagcaagaaatcttgatttatcaagatgaaacacatattccacgggtagtgacagagattttagagaactgggaaagtcagtctcaaaatcgtgagtttaatgcgatattgactgttgcctataaaaagcgtgtgatcgactattataaagagttcaaaaagcaactagctgaacgtgatggaagtttaaatattgccatgactttcagctttggcaatgaaaatgatccagaaaatccagcacctaaagaagctgcgatgatgtttaaagattatagccaatttacaggcattgaatttattgctggagacaagaagcacggtgaagatgcttattttgaagatgtggttgagcgtgcaacacgaggaggtagtggacgaaatcctaagaatatcgacctagtcattgtagctgatcagcttttaacaggctatgactctaagcgcttgaataccctgtatgtggatcgccctttgatgttgcaaggactaattcaagcttactcaagaacgaatcgagtgtttggaacagctaaggagttcggaacgattattaactttcaatatcctaggataaccgaagcgaccgtcaatgttgccctaaaactttatggcagtggcggaaaaagtagtcatgcaatcgttgaccattatgacattgcagtgagcaaactgaatctcttgcttggtgatttggtgttaaccttgccagaaccttcggagtgggctgatataaaggatgatgaggataagaaagaggagttcaagcaagctttctttgcggcaagcgaccaaatgaatttggtgcaacaatactatgagtatcagtggaatgatgaaatctttggtatgactgaacatacttggcttcagtatatcggtgcttataaaaacctgtttccaagacaaataggaccagatgaaccgtctataataagacagctccaaggaaagacaaaactgacgaacattcaagtgattgatgctgctcatattttaagtttaatcggtgcaaaagttggttctacgaatggtattcagacagtcgatgaagaaacaaaacgtattatttacgaacaaattcaagatctaagcaatatgggggaagccgtaactgctaagttacttcgtgaatttgtagagaacgagttatttacggggcatctttcttctagcattgactttgatgagacttttgataattggaaaaaagaaaagttgcaagaaaaaattgcagaattcgctgatttttgggggatacaggataaggatactctaattaggtctatttattcgtattcagatagtcagccagagattgtcccttacatcgaggaaataaataatagcgttaacatagaagcagctcaaaataaagattttaaaggtcgactaaagcacattatagagttaggaaaagcacttcctgactggatgcaagaagtaaaaagtagactatcataaaaattcaaggtaagtaggtttgatttacaccatacaagcacacgaaatacaaagaattgtgagaataacagtccctataacagaatagaacaatagttaattgttaaggaggagagatttcgcaaaataaaatctgattcaaagcgagtcagattttttaatttcatttgcataatcaagtcacttacgtattttttatataaaaataaaggcaaataaatatagccacgctgtgcgaaaccgatttcttttgatataatcagagatagaatatggtcaaaatgatcacatcataaaggagtattgaaattgagcaaagtaaaatgtccgaattgtggcgctgaaattgatattgacgatattttggcgcaagatatcaaggctcaggtagttgctgaagagcatgaaaaacacaacaaagagctcgaaaaaattaaaattcaagctgagaaagcagccaatgatcagttagccgctaaattagaacttgcagagcaaaagaaaaatcaggaacttgaccttgaacgcgaaaaaattaaggcagaatttgaaggaaaatccaaaaaggaacttcaagaacgtgaactcgccatgaagcaactcaccaatgatgcggaaactgaaaaagcggataataaaaagcttcgcgaacaattgtcggaaatgatggatcaacttcgcgaagagaaaaaatctcgcgaaaatgtcgaactggaagctaaaaagaagctttctgaggaagaagacaaaattcgcgaagaagctaagaaaaaggcagatgaagagcatcgcttaaaaaatcttgaaatggaaaagaaacttcgcgatacacaggaagccctcgcgactgctcaacgtaaagcagagcaaggttcacagcagaatcaaggtgaagttttagagcttgatctcgaagaaagtttacgtcaagaatttccgcttgatgaaattaaggaggtcaaaaaaggagttcgcggtgcggatgtgacgcagattgttaaaaacaatcatcttgaacaatgtggcattatgctctatgagagtaaaaacgcagcttggcaaccgaaatggattgcgaaatttaaggaagatattagggaggcgggcgcaagtattggcgtgttggtttccaaggaaattccggctgattatggagaaatgaaaaacattgaaggcatctgggttgtaaaaccgaagctagttttggcacttgcttctgcgatgcgcaatcaaataatttctgtatggacagccaatcataataacgaaaacaaagatgagaaaatggaaattctttatcaattccttacaggcacagagttcaggcaccgtgtggaagggattgttgaaaactactcaactttacaaaatgatcttgaacaagaatatcgactcgcaaccaatcgtttccataaacgtcaaaagatgttacgtggtgtaattgataatacaattgggatgtatggtgatctgcagggaattactggtggtgcgatgagcgagattaagcaattggaagagccagaggaaaattgagttgatttagaataaaaagaggattatagacaatgatttcagacgatataactacttgggtaaatacattaccctattggcaaagactcatttctgacaaaatttttcattctgttttgattgatgaagacgttatgaatgaaatttataatgtatttaagattgaaaacaagttgatgcaaggtaataatgaaactttgattttagagccaacgcagtttgatgatatccaacctgatgaaactgtcctgtggaaaggcatagaggacgttaaaggaattaatgctttatcagataatcagaaaataaatattggaaatcaactaacgattatttacgggaaaaatggctcagggaaatcaggatatgtaagaatatttaataacgcctttgtgtctagaggagacaagagtctactccctaatatctactcttctagtagtggtaagcaaagtgcaaagttattatttgaagtaaacggaagcaacaaaattgtgaactttcctaaagacatcaaggtgcagaactattgtagaagagtttcagtatttgactcgatgagtgcactaggagatatgacaaaggaaaatgaattatcgcttactccaatagaatttaatttttttgataagttggcgaaatttactaaagaaataaaggataaatttgataatgagaaagccttaaatcaagttaagaatgatttttccgaatcctttagcgaagaaacgagtgtcaaaaaaacgattgaatctatcaatgggaatactaagttggaagatatcaaaaaggcgattaaggtttcagatgatgaagatgaattacttaaaaaatatactgtacgaaagaaacagctagatgcgctcaatatcaatgaaagaataaaaaaaattaataatttggtacaagatattcatcaaatcaaagataagataaaagttttaagtgataaactctgcgttgaacgaatacaaaaaatacgtaagctaatagataagtataatcttcaggtccaactatcttctgaagagggacttaaacaatttgaaaatgaccaaataaataatttgggaactccagaatggaaatcatttattgaagcggccaaatcatattatgattctatcgagaaaggcaatgaaaaacttgattattgtattttatgtaaacaagatatttcaaagattgatttaattgataaatactgggcatatcttaaaagcgagtcagagttacaattagctcaagtaaatagagatattataagtctaataaatgattttgaaaaattgaaaagcaatgaaatcattattcctaaaacaaggctatatgaatatttagaactaaatcacgttaatatcttgtcagcaattactcaattcgagggtgaatgtaaaagtcaaactaaggatattatcaacgcactgaggaaaaaagaatttggaaacgaagttctatctccaacctttggtaataaaattttggatgatattattcaaaatttgaatgacgaaaaagtaaaactaaattcatatgaaataagcaaggaatctgagactatccaaaaatttttcaatgaatataatgctaaattgattgcagaaaaacttttgccaaaaatagaagattatattttaaaaacttcatggctaatcaaggcagaaaaatgtcgggtttctacacaatctatcacagtaaaacaaaatgagttattttcaaagtatgtaacagaagattataaagaaaagtttaagtcagaatgtcaagaattaaaagtgaatattgagattgattctgttcaaagaggaacgattggttccacaaagaacaaactatctataaaagggaaaaacttggataaaattttgagtgaaggggaacagcgttcagtggcattagcaaattttcttgctgaaacagaaatatctgaagaaaatgtttgtgttatttttgacgaccccgtttcttcacttgattatgagcgaagggatcgtatagcaaagagattagttaagctagcaaaaagtaagcaagtggtagtgcttacccatgatttatcttttatgcgttccctagaggatttagctaaaactgacgaagttattcttgattttcagcatatacaaagacttcctaataaaactataggagttataaatagtaaaatcccttggattgggatgaaagtacaaaaacgactttcagctttaaggctagatctccaaaacatgaccggcaactatagaaagtgcgatactccagaaaaagaagaaaagtatgtaaaagatgcaaaagcttggtgtgagaatttgagagagacatgggaaagagcaattgaagagacgttgttatgcggtgctgttgagcgttataagccgtcaatcaacacgcaaagcttaaaaaaagcgactttcactaaagaattatatcctgaaatagacgctgggatgcatgaatgctcagattgggtgcatgaccgatcattaaatttgggagagaatgttccagaaccagatgatctaaaaagatatttagatagatgtgaaacttttgttggatcgaacaaggctcattaagtgtagactgtcataaggctgaatgatgtgttgtgttgtaaaccacttttcttgtatatctactcaaaacttgtataatgatgggctaataaaaatgtcctagctcacaaatacgagttgggcaatgtggtgctattaataattaagacgcttgagatttttcaagcgtttttgtttgtatttatttcgtaaaaacaggcaaccgcacttggggaaatttctcagatgagttactatacttttgacaaaataatagaaagggtgaaaataaacgaatattcccattccattgataatactaatttctagaagtgttgaaattgtgattgggagtcataaattaaattatcagatttaaaaataaaatcacgattctaactgaatcgtgatttttggggctctttgtcaaatcctgtgggaaaattcttccccctctaagagtaagtcaccgcggtggcttatttttgtttcatcaattcatcaaaataatccgcttcttgaagttgctaaagcggcgaaacccgaaggcgacgcgcttgagcgccttgattttgttgttcatgccctccacgcagccgttggagtagggcgtgtgcagtgccaggcggatagaagtctttttggaggcaagaaattggagctttttcacaaaggtctcgggcaatagttcttctttcaattcgtcaatgatctcgaaaaacttctggtcgtcgtgcttcctgaaagctgtcagtgcggcctgcaagacttcccacgcctcgtcgagcacgggtgtataagctttgagttggagcaccaaatcatgactcgacgtccactggcgtaagctgcgggaataaaagcgattttcagagcagtcgtcaaaattcttgagcaaatagttccaatagtacttgaggtggcggtactgcgggctgtgcgtgtcgaattgcttcatctcctgaactcgcagctggttgaacgcgcgggtcagttgctggacgacgtgaaagcggtccgtgaccagctgagcgttgggaaaacagcgtttgatgagcgcaccgtaattggcattcatatccatgacgagcattttgactttgagtcgaacttgtcttgggaacttgagaaaataagtcacgaggtgctccaaacgacggtcatccagcaaggaaatcagtttgtgggtctgtccgtccatggcgataaaagccatttttcccttgcaatctcctgtggctttaaactcatcaatgcaaagcacttcgggcagaaagctaaaatgatcttttgtgggcatctggttcaaaaggcgataaaagctataaggcgagaccttcaaatctcgcgctgcttctgagattgactgcttgcttttgagtttcaaaataatttcttttttgagcaaaacagaatctttagtgcctttggagaccaagctttcgggcaactcaggggtcgacactttgccgcagaaacaacatttgtagcgctgaatagcaatgatcaaaaagcaaggaaagcacagatattctgtgagctgcacctttcgaggcatcatgccgtgcttatgaaatttgcttttgtgacaatgcggacaagctttaaaggtggctttataggtgaccttgagctcatagcacttgccctttttgaggggcatgagcgtctcgctttcccaatcctgagcttgcaaaaacaattctttcatgatagaatcaaacatagcttatctctcctctgtgtgtagtgatgtacttacagtttagaggtaagttttttattttacaagaaaacaaaatcctatgagaatgatttcccacaggatttattatagaaccgattttccttctaatggcgaaacaatcacaaatcggacaaaagttttgtttacactagaagaagaaattcgtaagcaataccagcttttaatcgaatatggtgaaattgaatcagaactttacaattttattaagaatacaccaaatattgcaaatcttcctagcctagtaaaaaataaatctgcctatattactgatgatccaaaggctaaaaatattttaaaattattattttcagatcaagctggtatgggatatatcaacgaagaactcaaggaggaaaacttagtaaatttgcttcttaaacatgaagttaaagtttcaaattttcataaatttcagcaagtgaatttaaaagttcttcaagaaaaagggattataaaagaagatgtagaaacagaaattctgaaaataaaaaatctcaatcgttttaaaatttttcaaaaaatttggagttacggcgtagtgtgttccccacatgaagatataaagcttcagagagaactaaaaaaaatggaatctgaaaagttgataagattcagtgagaaactttttgcggaacaagagtctgattttcttaatttcgtcataaataataaactttttgataattcttgggcgataagaaataaatacgaacatggtgcaccaatttacgagaataaaaatcaatatgaaatgtagtgacccccaaaatatagactttccactccaaatgttagatttttggtctaacttttgggggtcacatcaactttagcgagcgacgttattttatataatgtataaatgtttgctaccgttcttaaaacggctacattagacctgttgacgcaggtcttttttacttgtacatttattatatcaaaataatttaatcaaataaaaactatcatctttaattttttgtactagattactatttgtactataatgagtgaaaaaggtttgtgagataaattaggagataagaatgtcatttggagtatttttgttaatagcgtttgttatagtaaccattgcaagttttatctggaaatatagaggactaatctattttgtaggaatagtatttttaatttggttattttttaaatttttctttgtagctttaatcgtcattttagggcttataatagcttattttataagaagagtacaagaggatgagagaatatcttctgaagctgacagggcaaaacaagctcatcaagaggatgttgacgcatggagaaaagagcaagagagaaaatatggccctaattggtatcaagcaaatagagatgaacaaaaagctgaagcaaataatgctagaaataatcaagcaactaaattaatagattatgatagacgttgggatagtacagacccttatattatcttaggagtaagagaagtttcaactttttctgagataaagaatcaatataaatttctttcaaaaaaatatcatcctgacgttgtaactgaagcaaattctgatgctattatgaaaaaaataaattgggcttgggatgaaattaaaaaagaacaagaaaactactaaactgttgaagtctagtagtttttttataagttcaaataagaattattgttgtttctgataatattcattcattactaattgttctaagacagtatttttagttgaatgggcatattctagttcgttaattaattcattcatggataaattttttaaaaaggaaacacaagcgttttgatcgataacaaatccacggcttatttttattaacataattttgtgttttctaagttgattatttttattattgacaagatgtttaatttcaatagtcgttttataatcgttatctctgagcttcaaaacaagttctaatgctttagaattacttaaccctaatatatgatgtaaataatttaaaagtctgctatgtagtcctttaatactcataccagttatacttttaattttgtcaaaagaataatttctagttaaaagcatttccagttttggagttgaacaaaataagagtgaagcgataatattggcttcatcttcaaatggtaaaacgtcatcagaatattttcctgaaaattttgaagtaaatacctttttattatcttctaacttatgaaaatataagtgagtaagctcatgtaatatagtgaatttgattcgagttaaaggcattgattgattaataaatatcaatgttctatcttttttaggcattgttaaaccagaagttctattcactaaactatcgtctaggatgataaaatcaggattctgtacaaggtctttatagataatttcatttttttgagattcaaaaaatgaaaaattaatatcgtaattggactcaaagtataaaattatatctttaggaaagatttttgatactggtatttgactttgagccgaaatttgattaagtagttcattagctatatttagacacttattataaacgttagaatcaacaactctatattcaacttttttcatagagtaatattattttttccagatagaatcgtcattcaatcgttctttagcatatttcattaagtctttaacagcttcattgaattttacttgttcatcaggggtcatgtcttcacgttccatacgaaaagctccgataagctcactttcttcttcggataaatcttccttatgttttggctcatcttgacgtccgagtaagtaatctacactcacatcaaagtattcagcaatttcatttaaacgttttgttgaaggatttgaacgtttatacgttgttaaagtatttctaggataacccagttctctttcaatttgatttatggattttccggattttttaattagttttgtgagttgttcgtagagcataatttgttttctccaaaaaatatttaaatttttttatttaattgttgacaagagtagaatgctatgctacaataaatagtgtagaaaattattctacataaaaagaagcggaagcgcgctgaggcaactcccactaaaatgttaaattaataacctaagacattattaattttaacatttttaaaggagataagtcaatgcaagcactaagctttaaatcaaactttatacaattgaatagctgtgtgttatgtcacgtggcaggtgcttgttccatttatctaacaagctaggggagagagccgatgaatgcaccccgataaccgaaaggtcgtattgtcgtgagacattctgaatataccgtttaagggggagtatgctgtcaatgagagacactatattctctcttttaatttaaatatgaacaaataaagtttaaataattgtccgagatgacaaaaactagtcgcttaaactgtcagaaatggcagttttttattgtagaaaaataatggttttagtaaaaatctatcttaataaatttaattcgcttagggacttgttgatactgtccttaggaaatttgatttataactaagatagatttttttatgggaatataatttaataggctagagtaattacttttataagtaatggtactggttcaattccagttgttcccttaatgaaaaaagaatggagattttatgggaagtttttctgaaaggttaattcatttaagacacagcaataatttaacacgacaacaagtgagagacagtttaaatattaataatgtatcaacttataatcgttgggaaaatggaagtagaacacctgataatgaaacgattgtaagatttgctaatttttttaaatttactcctatgtatatgctaggattagtcaccttagaaatggtgaggaatatttactagatgttttagaaaatattgaatctgatgatactgttgaagggttgaaagcaaaagctggtttagcataatatcgaaaaaattacatgtagtaatatatgtgttacgttaatcaagaaataaagaaacaatgttaatcaactttaacaaactgcttgacttgtgatataataaacttaaattagactaaagataggaatgtatttatgaataagaatttgaatcttttgaattggactaaaaaacagcaaattatcggtggagcagttacgattgttttaatcggtggtgttactactggagtagtcgctaatgctaatcatcaagcaaaagaaagaaaaattgaagctttaaaaaataaaaaaattaaactttgtaaggctagagccgaaaaagtaaaacagctttaaaaatctaatgaagcagacagtaaaaaattacttgatattgctgaaaagactcctactgacaaaaatattaaactagctgaagattcaattcttaaagttaaaaatgctgaagtaaaaaaagtgtttgataatcaagtagttggtattaaaaatcgtgtgaagtcagaaaatgaagcaaagacagccgttagtaactatcaaaaagatgctatgaatcaaaataaatataaaacggctcaacaagcaatatctaagctcacaagctcttattcaaaaggtttgaaagatagcttgaataaaacaacttgcgacttctaaaaaacaagcagatgacgctacaaaagcacagcaagctaagagtgagagtgattcaaaagctaaggcaaccaatactaataaagaacaatcagcttcaactggaagtgcaactaacaataatgctaatgttgatgcaggtactgattcacaaagtacagtaaataatgatgttgctagtggttctaattcttacggtggaggaagtcaaaataactattctcaaggtggttcaaatacttctcaaaacaatactaattcagggtcaagtaatcaaggtcaaggtaatagcggacaaaataattctaatacaaacacaaatacaggcggtaatggctcgaatacaaataataatgggtcaaatggtggcaataacaataataatcagccacaaacacgatatgttggttgggtatcagttgacggagtgagaagatattctcaaactttcaatactttaggtgaagctcaaagctatgtagatgctacggtaaattcagctgaagttattaatttaggatttgaacatagtataagatatggtgttgagcctgcataaaatattttatttatttaaaaaaatcactttcttaaatgagagtgatttttttcttttagcgaacgaattagtacggttgaatggggttcaagtccccacgttcgcattaattgaccgagatgtcgttaaactaataaagaaaggtggaaatatgattaaaattccaattaacgcaacgaaactcttaggttcaaaagttactgtcgataagaccattgaaccagtcgcaaaaacatctactgaatcaggttatacgaaatatcgagctacaagtccgttacaaccgcaaggttttgaattacgtgtaccaaatggtaaaggggctaaacctacacgccgtcaagaagttgttttgacagatgtcacggttgcttatgttcgtaatcgcacaccaaagggcaaatattcacaagaatatgttgtttatgctgaagcgctcaaattagcgtaattatagggggggaattaaatgtcgttaaaattaagtaaagtttttgaaaaagaaaacaagttgactgaagaaattggtgattttgacaaagcattaggtaaagttttgttcttgcatgctgaaccagtaatggtctttgaggacggagaaccaaatgaagaaactggggaaatcaaacgttatccaacggacaaagttgattattatgaggtcaacgtttattctgacgcacaagacaagcaaattgttgtaaaaatgcctgctgaagctgatttttcagggcttgaatatgaagatgaaattaaattgacgaatcgctcaattaatttttggtctgataaagaactcatacctactggaaacggacagttccgaacgaattacttttcaggtgaaaaatggaatgctgacgggattgaaaaattaggtaaatctaattcaaaacctgaacaatcttcaaagcctgaagctaaaaacaatgaaccgaagaaatgataaatggtaaaacgtacaataaataaaggctctcgtttaaaacttagagataagtatgaccgtgaattttttgtattgattactggtttacttgtcgctagtcctgtagggctcttttataaaaaatttgaaaccttgccatcaaaagtactagcaatttatgctgtaggtattttgctcattgcattttcggtggcttttggtttattgattttgctttatcacaagtttcttttcttttcaaaaaacaattataaaagattgttgctgaactatgttgtgaagcatggtttagttgaaaaagaaacggttaaagatgaaaaaggaagtcatgaaaagttaaaactagcgcctatttatctgaagcaacctaacacgtatgaattacatacatattttccgattgacggtggagttcatcaagaaaaatttcttgatttagctgacggtttagaaacaactttttttgctgattatcaagaacaaaattttattaatgaaagtaaaattctttctaaaaaagcatttgttgagtatgtttttgcaattgacggagaacgtaatcgtattccagttaatgatgttatagttgataaaaagctaggtttaaagttaatgaatggggtttattggaattatgaagctgacccacatctgcttattgcaggaggtactgggggcggaaaaacagttcttttgatgtcgattttatcagcccttgcaaaagtaggtcacgtggatatatgtgaccctaaacgttctgactttgtgggaatgagagatgttccagtttttgaaaatcgtgtgttctttgataaagagtctatgattgagtgtttacgctcaaaaatgcaatttatggacgaacgctatgattatatgacaaatcaccctgactataaggcaggaaaacgatattctgattatggtttgacacctgaatttgttctttttgatgaatgggctgcttttatttctagtttagattttagagaatttgatgaagtgattcaaattttaactcaaatcgttctaaagggacgtcagtcaggggttttcttgattcttgccatgcaacgtccagacgctgaatacctcaaatcagctttacgtgataactttatgaaacggttggcggttggtcgtttgactggttcaggttatcgtatggtctttggtgatgaaaatgagaaaaaagttttcaaatacatcaagggtaaaattggtcgtggttatgtcgctaataatggtgaacttgcacgtgaattttatagtccgtctgtgccatttgataaaggctatgattttcatgaggaactttcaaaattaccaattttagctgatactactgaagttcagcttgaagctccaccaatttctaacgttgaacaggaacgagcagaaagtggggttttggaagaaaaatcttatacgattacctctttgtctcgtaagttgggtcaaccgtcaaaaactgtgaaaacagccatagagcgtttaatatgtgacggatattctataggtgaaaaatcaccttacaatgaagatgattttattgctttacaaactgttttcttaaccaaagaaacagaagaatgtactttgaatgaagccattgatactgtcctagctgatgaatcagagtttgaaaaagaatttgtgacatttgataaatctgcatgagatttttgcttataatttatattttgatatactattttatataggggtataaataatgagtgatacatgtactatttcatttttaattggtaatggttttgatatagcaatattgaaaagtttagataagaattttactacaacgtatggagaattttatgattatttggcttattttcttactaataaagaaaatgatatttataaggctattgataataaaaagaaacaattctcaaataatgaaaatgaaacatggaaagattacgaacttttattaaaagaggttgtatgtacgaaattaatagaattaaaacaatatactgataatgcattgcaggaaaaagtatttttatcatttttagaagattggaaagaaatacaatatacatttgctaactttttaaatcatgttattactccgcaaacattgatggatgtaacagatatggatggtaaaacgactttacaaaaatttttgggcgacttgcctatagaagagtataaaaaaaattgagtttccccaaaaaacagataactatattaaaattgattatcatatttttaattttaattattctacattagcagataattatttttattggttatttaattaccacccatataatgtttcaaaaaataattcacatttttatccgaatcctagtaacatttcagggggaaagacctcaactaatagtgatactaactattttattcgttcaagtattaattttcaccaccctcacgggcaactatcaataccagagtcgatattgtttggaatgagctataataaaaaaacttattgtaattccaactttgaaggaaaattatctcaaaagttagataaagcatattggagtatgcatacagacaaagtaaaaccatttatacaaaaaacagatttgttcgttatttttggtcactctattggagaatctgaccaatgctggtgggaagaaataatagaccagttaaaacaggggagtgaattaataatatacgactatgaagggaaaggtcttaagaagaagatactacattattgtaaggatgatgaaaaccttataagtgacaggatatttgtaattgactttgatgaaactaagcatttaaagtattcatttaaattttaagaagcgtaaatttacgcttttttatttgattttatatttctacctgcaatcggcggtgagtaaatgataggaacttgtttccgtttgtctttagacaacttgccgtaggcaagtcattttccacggtcgaattgccttcgcaaacggtagtgcgcgatagcggtttattcttgaaagaattggaggaaaaacttttggattttattttaaggctattgcgttttaaacgtaatgctttttttgaaaaagtgcaagaacgtaagcatagctccgagcgtagcgtaaggagtgagcgtgtgattgcacggctgacggcttgcctgaccccccaaatgctaatgagggggggtacaagtgatacaagtctaactatcctagtcatatcaaggctttaggttgtatcacctctgacttatgtttagaaagtgaggaacgaacgattgaattaaaggatatacgctctagcgcaaatgttactcaacaattcatggctgacttacttggtatatctttagtctattatcgaaaaatggaaaatggcgaccgtccactctctaaacaatttgaagaaaaaatcagaaattcttttttcaaaaaaagagagagttcgacggtctttgtgggtacaaatgactatacaaatattcgttttcagactctaaatgttcgtgaagtagtttctaagattttaggtttaaacgttgagaattttcagctcaatgagtataaccgttatcaataccctttctatattagttacggtcacattaacgtttattatcacgataaagatataaaagcaggtgtgctaattgaaatgagtggtaaagcgtgccgtgaaatggaatatgagtttaaatatcatcaaaaacagcgtacttggtacgatttttttaacgattgttttctctatgccaataaaaaagcaccagataatgatgattttgtaaaaatcacacgttttgatttagcacttgatgaacaatataatccgcaagaaggaaattttgatttgttcaaacttttaacttctgcccgtgagggtcggtggaatggtcgcaaacaaaactattctgcggttcttggtggaagaaggacaaaagaaggaatgattaatgacggtctaactgtttattttggatctaaacaaactcatttattttttcggttctacgaaaaagattttgagagagcaagtcaagaaatgacttcggttgaagcgatacgtgaaatgtatggtttgcgaaatcgttatgaaatttccatgcgaaaagaaatatcaaccgactttataaaaagatatatcgaggaagattttgatttagccgatgaaggggtcaaaattattaatgataatctgacattttatgataaggaaggaaacttagatagtgaatggtacgatatgatgggtagaatggatgcttatcattttactgttcgtcctgaagtgcctgatttgaaccgaaaatatacatggtttgaacgtggtgggcctgtatcaacttatctattgttaaaaaaagctgaagaactgacaggtgaaagtcgtttggaagaaatttttaatgaagcagaactcacagaaagacaagaaaagttcttgaaggaatttagaatgataaggggtgcaaatggatagcttattcaaattgacaggttttccagttgatgaaatagtaattaaagttatcgtagctattattgtatttgtggttgttattgcaatacttgcggatatatttgattgataattagttaaaggaggagagaggggtgattagacctaatgtagaacattttcaaaaaagaatgttaagagttccaaacaacgtccgaatgctccagtataaaatgagtggatatgaatttaatttgaaaaaatttgagcagggagtttatgaccagctctttttttatattccaaatgaaactgaagcgttgggattgcttaatgaaataggcttgatgtttccgcctgattcaatgacacgtaaaaaatattattcactctatcaagataaaaaaattgcggatttgcctgaaggctataaaatggcaatttattttttaattgataatgatttagcacaagtcactttatgatttttcataaataaaaacaaagttatttttcggataaggaaaggcaaattttgtggtgggaaataaaaaaagaaaagagataaaatatgccaactttaaaaacagaacttaaccaaattacaactgatttaaatgaaattgaagctgttatcaatgcagaaaatgcctttcaagatgctgctcaaaagctcaaaggaaaagggaaagcgctccgtattgtagaaaatgatacttctatttcagatgatacagtcgaagttattgcgaaccgtacaaatgacttgattagaaatcttgtcaaggcttcatcatcatacaatttcacaaagaaatcatttaatgaattaaaaggaaaactggaagaagcgcaagcgcaagcacgtagtagcgctaaataaatagtccacgtaaatctttgaaataagggtagggtttgcctttccttatctgaaaagtaacaatgttaaaagcctcactcatggtaaggcaagagaaaggatactcaccatgagtgagaaatagaaatatgacagtaacaaatatgagcaaagcacaaccacagattgatttttcacttcacaaagcaccatataaagatttgtatgatatacaaattctttttcctgacggtacaaatcaattttggtctggaattaatgaaaacaaagtaatgtggctgtgtgagaaatacaatcctatctcaactccgctgtcaattgcgattgctcctagatttttaccaaaatctaatattcaaaaacctaactgggaaattagaaagggttagaaatgaaaaaagatgattctttattaaaaattataatagtgtttttgctttcgtttttgtgtcttcctagacagattataacacactcatttgtgtctacttttatagtatagctccaaaaatgaaaaaaacaaaatataaatcaagaactccgccaaattattttggtgagatattattttaaattgaaaaaagaggtgaaaatgtacgttgtaaaaatgcgtggtggctatctttgtgccaatggtggagcgactaaacacttaaaatttgctactacttttgatactaagaggaaagctgaagaagtggctgaaaaatggttaagaagtgatgtatcttttaaagctgttgaaaaagaaagtgaggaatatgagcaaaattaaaatttatgaattagctgacaagttaggggttgagcgaaaagagttactctctaaggctcaagaatttggaatttcagctaaatcaacatcaaaatctttgagtgacgctgaagtattaaaactagaaaatttctataaaaaatcagatactcaagaagttgttggcgaagaagaatctgaagtggaaacaattgatgttcctaaaaaaaaggaatcgaagttttctctccttgcaggtagaaagcctgaaaagaataagccaaaaatgttaaggaagttgcccaatgaaacaccgcctaagcttaaaaggatttcagctaaacaggtaggggctttggttgtgggtggggtcggtttcttaatattgtcaaacgtggtcttgtttggtttgatggcaagtggatacagaccaacacagaaaattatttatcaagaagtgagtgctacgcaaaaagcttcaggtaatggacttgatttacaagcgaaaaattatcttgatggatttgttcaaacttatttcactttccctgaagatgaaaaagaccaagaacaagcagttaaagatattaatgcttactttgttcaaaatctaccagtgattagtcaaggcattcaacgtacaccttctaaatttgagggtgcggtcatgatgagtttgacggataatgaagcgacttataaagtgacttatagtgcaggtgaagtcacaacaaaagaagtcaaaaaggggaaagatacaacaaaacaaaatgtggtgaaatatcatgatgaaacaactttgtttacgattccttaccaaaaagtaggttcagcttactatatttcagatgaaccttatttttcaagtgttcctgatttacaggcaaatgaaaatcaagtacctactaaaacatggtcaggtactgataacaatagtgcttcagtcaaaaaagatttagacaagttcacaaaatcattgtttactgcttatacaactgacggtgatacattgaaactaatttcaaaagggctttcgcttaataaggggcaagagtttaaatccttagaccaagccacttatgaatcaaaaggtgacaataaatatcatgcggtagttcaagtcaccatgaaaaatgcactcggaacacacgtagaaaattatcaatttacaattgaaaaacaaaagcagtcatattttgcgactgactttaaacacactttacctgaaggtaagaaagaataggagaaatcaaattgaaagagatactaattaatcatatcagcgtaacttatggtgcgattggattgacaggcttttggaactttttccaagaaaatgcaaaatatgtcatttttgcaggagggatttattttgcaggtaaagaatggcaaaagaaggcaacaggtaaaatgataatgatgattgttgttgcagcagttttgtttgttctcacactagaccctcaaacggttttacaaccactcggtcaaaaactatttgaaatgattggtgctaaataaaaaggagttatgattgtcaaaagaaaataaaaaaggactgaaatattctaataaaagttcactttctcaaccattaagaatccaaaaactttttgaaggatggtcgctaggtaaggcttggcggttgtccttttttatttactttggattgattggttttgctttgtggaaacttttattttcaaaatttactgtttttcctgttggttttcgattaatggtgctagggataatttgctataaaagtgcattatttatctctgaaattcgtcctgacgataagacaccacctgtttattttaaagatatgattgtatttttatttaactttggatttacaggaaaaagcatacataagggatggattcatccattaagaaaaggaaaagaggaattagaagaaaaatgaagaaaaaagcaatatgggaaaatcacgtattatcggtgcatgataatttagttttaaaagatgatggcacagtatttgcaatgtttgaagtgcctgcgtctattatatcaatgatggataataaaggaaaagaggcacacaaacaagcgacacaagcagtcattaccagtttatacgaaaatttaggctttgaaattttagtgaagccactttcaaaagatttacttaaatcatatgaagtattattaaaagattgcgaccctagaactcgtgaatttggagagtatctattagagcagtcttattcagaattaatgaatgaattaggaaacttacatcattataaattctttttaactgttcctttgagtgatttctcattttcaggtgatttaagaaaaatgatgaaagaaggctggcaacgtttcggaaaatctgttgtaaatttgattcgtagagaattagaattcaatcttaattggtatgaatcctataaacaaacggcttcagatttagaaatgaatttaaatttacttcaggctcgccctttgaagagagaagaaacgctcctttataattcacttaattatatacgtggtattgaagtcaataaagatgaagtgttagctgatgttaaaaatgccattgaaaatatggatgatacaacgatagatgttcgctccgatggactattagaaattcatcatccacaaggaagtagcctattaaaattcttaccattggctgattatccagaagtggttaataatattcatcttatcgaacacttgcaaactttaccatttccagttgaatttcgtatcaaggctcgatttagaaaaaataaaggggctttaggaatggaagctaccgcagagagaaatcgagatagaattggtacggaactttcagaagcagatgaaaaaggaaatgtgacaaaatcttcatctgttggtgcgtatatgattcttgaagatattatttctggggtagataatggggaatatttcctagacttcttacctgtttttgcgattactggttcaactcctgaagaaattaattattataagaagatgttaatgactgccatggaaggtttaggagttaaaattgttcctagtcaatgggatcagccttatcttttttataaaatgcgttcaactgaagaattgacacgttcagacagatattgggttcaaaatatgaaagtgtctgcttttgttgaaaacttgttttttgtcagtcaaaaagtgggttcagatattggattttactttggacgtgttgaccataccactttaaattgggcaggaaactatgaaaaggcaattgcaagttcatctactccgtttttctggaatatgtttcaagccaataaagaagatattgaagggaaattaggagatagtcctcatgtgggtatttttggtgatacaggttcaggtaaatctttcttcgcaaaacttgcatttgtttatcattccatcttaaaagggttgagcctttatattgaccctaaagatgagatgaaatcacagtttttatatgtgagggataaattagaatcttatttgccagaaatggaagaaaaaatagcggatttagtcgctcttattgaagatgatgaaattttggaacgaagaattagaaatctagagtttgcttatcataagcctttgattgattatattaatcatattcattttgtcagtctaaatacaaaagataaaaagaatattggggcattagaccctattgtatttttggaaagtgaacaagcaaaagaactttctacacaagttactgaagggctgattggtacaaaattgactgaagatgatagatttgaaaactcatttaactatcatttacaacaagtcattgaaagaagagcaaaaggtgaaactgttggattgcttaatgtttttaaagaaatggcaaaatcaaaagaagaacttattaaagaccgttcggaaaatatcctttcaaaaatatcaggtacaatgctagaacttgtcttttcagaaggtaaaaatccgtcagttagtatggacgaccacatcaccgttttaggagttacaggacttaatctagctaaatctggtgaagtaaaaacagctcaaaataaaatgtcagatattatcatgtatgcgttaggtgatttttgtcgattctttggtgcaagaaatcgagaccaagaaacggttattttccttgatgaaggttggttctttaatacaacggatattggaaaaggtattttaatgcggatgaaacgtgttggacgttcggaaaataatttcttggctttgattactcaaagtgtaaaagatggttcgtctgatgaagatgatacagcttttggaacaatttttgcttttaaagaaactggaaatacaaaggcagttcttcaagcatttggtttacctgatgatgacgaagatgtgataacttggtatgaaaatcaaactaaaggtcaagcccttgctaaagaccctttcggtcggattggtcgtgttgtaattcatggtcaaaattcagcattgaatgaatgctttaaaacaacgtttagtgaaatggaatcagcaaaggcggtggcatagtgaaaaagaaaatgttgttatttgtagggctattagcattttttatgccttcagccctgcatacttttgcggatacaggctttactccgccctctactcctgatttttcagtttcaggtgataagaatacttccaaaaatgattcaaaggataaaacagattcatcttctaattcatcaataactgacggaacgattaaaccaccaattgataatttagatgattttggctcggatgattctccaagtaaagtcatgactgaagcggaaagaaaaaaggcaagagcaaatttagataattatacttcttatatgtattttgatgatggaaaatttctaacaggttcaatgaatgcaggaatgcaaacttttttcattcgttctcaattttttgttacaaaaacaatttatcgttttgtgaatgcggtcaatgaaaagttaaatagtgattcattaatcactcgatggacagcccaattgttcgtgacagttcaaaatatttataatcaattttctaaccctcaactttatccaattattgcgatagctgttattagtagtcttttattttactggtttaaacgtcgcttttttgaaggggtcagaaaattggcgcttgtgatgatactggtaggtctatttattaatggtggtcaaaagttaactgaacaagttaatactgctttaaatgatgttacaactactttgatgtcaacggttaaggtggcaggtgtttctgcgaaaaattctaatgatttaaaaacgacaatggttgaagttccctttctttaccttaattttgacaatgtgaaaataaatgcggatggttcatctaatatctctgaagataacattgtcaatcttctaacttcaggggatgataatgacaagataaaagcgattcaatctgatttaaaagattcacatttgacctcgaaaaaaatgggtgaaaaagtattaactgctctagcaagtattttcaatgcaatattagtcggttttatttacctagcttttgcgataatggcttttgtaatgagaatgtttttcttaattctcttacttttactgccatttgtggggattctttccttatttcctgtctttgacgtagtgattttaagatgggcaaaagcaacaggtggggcattgattatttctaatgtagtcgttattggtacagctttaattagtgttttagatagtattgtttcttctacagtaacctcaatgattgggagtgattatttctttatcacccttataaaattcattgtatatattatcttatttaagaaacgtcaaaaaatcttagatattttcaaagcaggacatcttagtaatagtggttttgcaggtcgtatggatggcttactttcaaatgttcgcagaaaaggctcaaatatgattaaagctccacttttggcaggttcttcagcaggactggtcgcaggtttaactgctggtcaaatggcaactggtaaggcgaaagaaatagctaaacaacgatttacaggtggaactggtagtttgtggcatggtggcttgagtaaaaaagcagaccgtaccatgaataaaacagataaagcaaatcctgatactaaaaaaggtcaaaaactacaggctaaagaagaaaaaatcaaacaacgtttggaaaaacgcaaacagaaatttgaaaatcctaatctgctaaaacgaaaagtagctgactatcgcaaacagtatcaacaacgaaaaactatgaataacccaaatcttgctcaaaagatggaacaaaaaaataaagaaaaaaatgcttcgattcaagctctttatgataaaaataaaactcaacttcaagcaagagcaattgagcgtagaatgcgtgaagctgttcctgaccaagaaaaatctaatctatcagaattagctaaaaataagcaaaaagctttagctaaagaaaagttagatgaaagagcaatgaaacaatcagcaggtagaatggctgaaagatttgaaaaggaaaaagtcagtcatagtagttttaaacgtaaaccaaactatccttgggataataatgataccaatcaaaaacttaacaatccatttgtgattgatgaaaaatagggggtgaaaatgaaaaaagcaattaagcggaaggttatattagttagccttccttttttgttgcttttattgccaatattagctttcttttcattatttgttggttcaaattcttcttcagatacagatattaatattaatacaccacagcaacaaactgcaaaagtgatttgggatagagttctaaaagaaggtggaacgaaagaaggggcggctgctttacttggtaataatcaagcagaaagtgaacttcaaccttcaattattcaatctaatgcaacctataatgaagcaaaagcaatggataccactttaggcggttatgctttcggcttggctcaatgggatagtggcaggcgagtaaacttgctaaactatgcgaaaagccagaaaaaatcttggacagatactaatcttcaagttgagttcatgattgagcaagacggtacagattcaacgttacttaaaaaattagtcaaaggaactaatgttaagcaaacgactgaagatattatgcgaaagtgggaacgtgcaggggcggttgatagccttccaaaacgtcaaggttttgcggaatattggtacacgttcatgacgactggtggtgatagtggaactggtggtggttcaggaattactccagatataccttctggttggactttagataaaccaattaatacaagtggttatcttgcgacaagttatgagtataaacaatgtacgtggttcacatggaatcgagcgaaagatttcggtattacctttggaatgtatatgggaaatggtgctgattggcaaaaacaagcaggatatactgtaacgacaactccgacactccatagtgcggttagctttagcggtggtcaaacagtaggcggtcaatggaacgcagaccctgtttacggtcacgtggctttcgtggagggaatacattcagacggttcagtcttgatttcacagtcaggaactggttttagtacggtttatactttccaagtcttaactaaagcacaagctagtcaattacattatgtgataggaaaataaaaaatagtcaattaatgttcggtattaacttagttttcacgttgaaaattctaaagtttttaaagttatttccgaacaatagaagatagaaaagagaaaaaatggaaacaccaaaaatttttgtagtaaatttaaatagttataacaatggaagaacaagaggtaagtggtatgaattacctgttgactttacaaggatacagtcagatttacttttagatgtggcgcatggagaagaatatgcaattcatgattttgaaaacttttatggttataaagtaggcgagtattcatcaattcaagaacttaatgagtatgcggaaaaattggaggcaatttctgacatagatcatttaaaagattttcttgaaatttatagcattgatgatattatcggcaataaagatgacttggattttgtggaagccgaaaatgatgaagatttggcacaagaattaattgaacaaatgggcggtttagaagttttaagtgtagaaacgttacaaaggtattttaactttggtgcttacggtcgtgatttagcgattggtgattattcaaaaacaagtcatggatatattagagatatttaaagtttaagtaaagagcaagaaaaatcttgctctattttttgttggaggaaaaaaatggaagaaaattataaagataaacggggaatttcagaattatttgatgtacctataaaaacacttaataatgatttaactgaaatgagacggactgaatttaatgtttatatattacgaccaagtcataaacgtgtttatataaatgtccagggttataaatcttttcttgaatataagcaaaaaataagagaaagtacaatttagaaaaaagataaaagtaatatgcagatatgatataatataagtgctgtatagctttcttttatctttggtaaaaggaggaaatatgttttataagcaacttgatagtggaaaatatagatactttgaaaaatactttgatgaaaagagaaataaatggcgacaagtaacggtcacattaaaatcaaaatcaagggttgctcaatcagaagctaaaaatagattagcaagaaaaatagagcaatcaaaaaaagtaccaactgctaacgagattcaggaacaaatcgttcaaaataagacggtacaagaaatttatgaagaatgggttgtaattagaaaacaagatgttaagccagcaagttttgtagcggagcaaatttctttaaaaggatttatcgaaaaattttcaaaatataaagtctcagaagtgacgaccgcagatatacaaagttatttaatggaattagatattgcgaattcaacaagaaaaaatcgcagaatttacattagaattttgttcaagtatgctgaaaacatcgggtatattgattcaaatccagctgacaaagtagtattacctaaagtaaggttagaaattgaaacattagaacgagcaaatgaaaaatttttaagtaaagaagaaatgagttctgttttgatattttgcaaatcttataaaaaaaatataagatacactttagctatggaatttattttcctaacaggatgtagattcggtgaatttgcttctattcgttatcaagatgttgatttcaaaaataagttactaagaattgaccacactttagaatatcgtgttgcaaaatatgatgatcgagttattcaaacacctaaaacggtaggttcgattcgtacaattagtttaagtaatcgttgcttggaaattattgattatttccaaaaaaactgtttagatgataagtttgtttttgtaaatgcggttggtggaattttcagacaacctgtattttataagtttatttgtgataattgtcaaaaagtattaggaaatgaaagaaaatacggtatccatttattgagacattctcatgtatcattacttgtggaacttggagtaccaattaaagcaattatggaacgagttggtcatagagatgagtcaatcactctgaggatttattctcatataagtggtacaattaaaaatgaaattagtcaaaaattgaaccaaattaatctctaaaataactaaccaagagactaaccaagaattaaccaaagcaaaaagaatcatgaggaagattagggaatatcaatttttgaaattcttctaaaaactattgttagagacttttgaggaagaataaggaagtataaataaaaat

SEQ ID NO: 5 - DNA, 978 bp, Lactococcus lactis
  source 1..978: mol_type = genomic DNA; strain = Lactococcus lactis subsp. lactis bv. diacetylactis SD96
  CDS 1..978: note = lactate dehydrogenase (ldh) from Lactococcus lactis subsp. lactis bv. diacetylactis SD96; protein_id = 6; translation = MADKQRKKVILVGDGAVGSSYAFALVNQGIAQELGIVDLFKEKT QGDAEDLSHALAFTSPKKIYSADYSDASDADLVVLTSGAPQKPGETRLDLVEKNLRIT KDVVTKIVASGFKGIFLVAANPVDILTYATWKFSGFPKNRVVGSGTSLDTARFRQALA EKVDVDARSIHAYIMGEHGDSEFAVWSHANVAGVKLEQWFQENDYLNEAEIVELFESV RDAAYSIIAKKGATFYGVAVALARITKAILDDEHAVLPVSVFQDGQYGVSDCYLGQPA VVGAEGVVNPIHIPLNDAEMQKMEASGAQLKAIIDEAFAKEEFASAVKN
atggctgataaacaacgtaaaaaagttatccttgtaggtgacggtgctgtaggttcatcatacgcttttgctcttgtaaaccaagggattgcacaagaattaggaattgttgacctttttaaagaaaaaactcaaggagatgcagaagacctttctcatgccttggcatttacttcacctaaaaagatttactctgcagactactctgatgcaagcgacgctgacctcgtagtcttgacttctggtgctccacaaaaaccaggtgaaactcgtcttgaccttgttgaaaaaaatcttcgtatcactaaagatgttgtcactaaaattgttgcttcaggtttcaaaggaatcttccttgttgctgctaacccagttgatatcttgacatacgctacttggaaattctcaggtttccctaaaaaccgcgttgtaggttcaggtacttcacttgatactgcacgtttccgtcaagcattggcagaaaaagttgatgttgacgctcgttcaatccacgcatacatcatgggtgaacacggtgactcagaatttgccgtttggtcacacgctaacgttgctggtgttaaattggaacaatggttccaagaaaatgactaccttaacgaagctgaaatcgttgaattgtttgaatctgtacgtgatgctgcttactcaatcatcgctaaaaaaggtgcaacattctatggtgtcgctgtagctcttgctcgtattactaaagcaattcttgatgatgaacatgcagtacttccagtatcagtattccaagatggacaatatggcgtaagcgactgctaccttggtcaaccagctgtagttggtgctgaaggtgttgttaacccaatccacattccattgaatgatgctgaaatgcaaaaaatggaagcttctggtgctcaattgaaagcaatcattgacgaagcttttgctaaagaagaatttgcttctgcagttaaaaactaa

SEQ ID NO: 6 - protein, 325 aa, Lactococcus lactis
MADKQRKKVILVGDGAVGSSYAFALVNQGIAQELGIVDLFKEKTQGDAEDLSHALAFTSPKKIYSADYSDASDADLVVLTSGAPQKPGETRLDLVEKNLRITKDVVTKIVASGFKGIFLVAANPVDILTYATWKFSGFPKNRVVGSGTSLDTARFRQALAEKVDVDARSIHAYIMGEHGDSEFAVWSHANVAGVKLEQWFQENDYLNEAEIVELFESVRDAAYSIIAKKGATFYGVAVALARITKAILDDEHAVLPVSVFQDGQYGVSDCYLGQPAVVGAEGVVNPIHIPLNDAEMQKMEASGAQLKAIIDEAFAKEEFASAVKN

SEQ ID NO: 7 - DNA, 1374 bp, Lactococcus lactis
  source 1..1374: mol_type = genomic DNA; strain = Lactococcus lactis subsp. lactis bv. diacetylactis SD96
  CDS 1..1374: note = UDP-N-acetylmuramate-L-alanine ligase from Lactococcus lactis subsp. lactis bv. diacetylactis SD96; protein_id = 8; translation = VIDKEIELLTENFIMEKTYHFTGIKGSGMSALALMLHQMGKKVQ GSDSTDYFFTQRGLEQADVPLLPFDEKNIKPEFELIAGNAFRDDNNVEIAFAHKNGFP FKRYHEFLGHFMEDFTSIGVAGAHGKTSTTGMLAHVMSNIVDTSYLIGDGTGRGIEGS EYFVFESDEYERHFMPYHPEYTIMTNIDFDHPDYFEGIEDVTSAFQDYANNIKKGIFA YGEDVNLRKLTAKAPIYYYGFEANDDYRAENLVRSTRGSSFDAYFRGEKIGHFVVPAY GKHNVLNALSVVAVCHNLGLDMTEVADHLLTFRGVKRRFTEKKVGETVIIDDFAHHPT EIEATLDAARQKYPDREIVAVFQPHTFTRTIAFADEFAEVLDHADTVYLAQIYGSARE VDHHEITAQDLADKVRKPAKVIDLDNVSPLLDHDRGVYVFMGAGNIQKYELAFEKLLS QVSTNLQ
gtgattgataaagaaatagaattactgacagaaaattttatcatggaaaaaacatatcattttaccggtattaaaggttcaggcatgagtgcacttgcgctaatgttacatcaaatgggaaagaaagttcaaggttcggactcaacagattactttttcacacaacgtggtcttgaacaagctgacgtaccacttttgccttttgacgaaaaaaatattaagcccgaatttgaattgattgctggaaatgcttttcgcgatgataacaatgttgaaattgcatttgcccataaaaatggatttccattcaaacgttatcatgaatttttgggacatttcatggaagattttactagcatcggggttgcaggtgcacatggtaaaacttcaacaactggaatgttagctcacgtgatgtctaatatcgtggacacttcttacttgattggtgacggaactggtcgtgggattgagggaagtgaatattttgtctttgaatctgatgaatatgaacgtcatttcatgccttatcatcctgaatatacaattatgacaaatattgactttgaccatcctgactactttgaaggaattgaagatgtcacctcagctttccaagattatgcaaataatattaaaaaaggtatttttgcttacggggaagatgttaacttacgtaaattgactgccaaagcacctatttactattatggttttgaggctaatgacgattatcgtgccgaaaatttggtcagaagtacacgtggctcatcttttgatgcttatttccgcggtgaaaaaattggtcattttgtcgttccagcttatggtaaacacaatgtattgaatgccttgtcagttgtagccgtttgtcataatcttgggttggatatgacggaagttgcagaccacttattgactttccgtggagtgaaacgtcgtttcactgagaaaaaagtaggtgagacagtcattattgatgattttgctcatcatccaactgaaatcgaagcaacattggatgctgcacgtcaaaaatatcctgatcgtgaaatcgttgctgttttccaacctcatacttttactagaacaattgcatttgctgacgaatttgcagaggtgcttgaccatgcggataccgtttaccttgctcaaatttacggttcagcacgtgaagtagaccatcatgaaattacggcacaagatttagctgacaaagttcgtaaaccagccaaagtaattgatcttgacaatgtttcgccattgttggatcatgaccgtggggtttacgtctttatgggagcaggaaatattcaaaaatatgaacttgcttttgaaaagttacttagccaagtctcaactaacttacaataa

SEQ ID NO: 8 - protein, 457 aa, Lactococcus lactis
VIDKEIELLTENFIMEKTYHFTGIKGSGMSALALMLHQMGKKVQGSDSTDYFFTQRGLEQADVPLLPFDEKNIKPEFELIAGNAFRDDNNVEIAFAHKNGFPFKRYHEFLGHFMEDFTSIGVAGAHGKTSTTGMLAHVMSNIVDTSYLIGDGTGRGIEGSEYFVFESDEYERHFMPYHPEYTIMTNIDFDHPDYFEGIEDVTSAFQDYANNIKKGIFAYGEDVNLRKLTAKAPIYYYGFEANDDYRAENLVRSTRGSSFDAYFRGEKIGHFVVPAYGKHNVLNALSVVAVCHNLGLDMTEVADHLLTFRGVKRRFTEKKVGETVIIDDFAHHPTEIEATLDAARQKYPDREIVAVFQPHTFTRTIAFADEFAEVLDHADTVYLAQIYGSAREVDHHEITAQDLADKVRKPAKVIDLDNVSPLLDHDRGVYVFMGAGNIQKYELAFEKLLSQVSTNLQ

SEQ ID NO: 9 - DNA, 2223 bp, Lactococcus lactis
  source 1..2223: mol_type = genomic DNA; strain = L. lactis subsp. lactis by diacetylactis SD96
  CDS 1..2223: note = GTP pyrophosphokinase from Lactococcus lactis subsp. lactis bv. diacetylactis SD96; protein_id = 10; translation = MPKEPDLTGAEVVNICSDYMNETDILLVEKALACASLAHADQYR ASGEAYFVHPTQVAGILAKLKLDAVTVSCGFLHDVVEDTNFTQGDLQELFGDEIAEIV DGVTKLGKVEYKSHEEQLAENHRKMLMAMSKDIRVILVKLADRLHNMRTLKHLRPDKQ KRISRETMEIYAPLAHRLGIASIKWELEDLSFRYLEEAEFYRIRGLMNEKRTAREALV AEVIGKLQERVEKAGVDAEIYGRPKHIYSIYRKMHDKKKRFDEIYDLIAIRCITETTS DVYTTLGYIHDLWKPMPGRFKDYIANPKANGYQSVHTTVYGPKGPMEFQIRTREMHQI AEFGVAAHWAYKQGIKAKVDVHEISETLNWIHELVELREEAGDSAEDFVKAVQEDILS DKIYVFTPNGEVQELPRGSGPIDFAYAIHTKVGDHATGAKVNGRMKPLSVQLKTGDRV EIITSSSSFGPSRDWINLVKTNKARNKIKQFFKNQDKELSVNKGREMLQEALEEGGFV PNQYLDKKHLDEVFNKISYRNAEALYAAIGFGELSATTIANRLTENERREVERAKQKA EAEELMKGEVKRESSKNIMKIRHDGGVSVSGIDSLLVRIAKCCNPVPGDPIVGYITKG RGVSVHRADCQNVRSMEDFEQRLVEVEWDDSENLVKEYVANIDVYGFNRPGLLNDVMQ VLSNSTKNLISINAQPTKDKKMANIHIALGIKNLSDLTLIVDKIKMTPDVYSVKRTNA
atgcctaaagaaccagatttaaccggagcagaggttgtcaatatctgttctgactacatgaatgaaacagacattttactcgtagaaaaagcacttgcttgtgcttcacttgcccatgcagaccaatatcgtgcatcaggagaagcttactttgtccaccctacccaagttgcaggaattttagccaaattaaaactggatgccgttaccgtttcctgtggttttttgcatgatgttgttgaagataccaattttacccaaggtgatttgcaagaattatttggcgacgaaattgctgaaattgttgatggagttactaagttaggtaaagttgagtataagtcacacgaagaacaattggctgaaaaccaccgtaagatgcttatggctatgtccaaagacattcgtgttattttggtcaaattggctgaccgcttgcacaatatgcgcacgcttaaacatctcagaccagataaacaaaagcggatttcacgtgaaaccatggaaatttatgcccctcttgctcatcgcttggggattgccagtatcaaatgggaattagaagatttatcattccgttatcttgaagaagcagaattttatcgcattcgtggtctcatgaatgaaaaaagaacagctcgtgaagcgcttgttgctgaagtaattggtaaattacaagaacgagtcgaaaaagctggtgttgacgcagaaatttatggtcgtccaaaacatatttattctatttatcgtaaaatgcacgataagaagaaacgttttgatgagatttatgacctgattgccattcgttgtatcacagaaacaacaagtgacgtttacacgactttaggttatattcatgacctttggaaaccaatgccgggtcgttttaaagattacattgccaatccaaaagccaatggttatcaatctgtccatacaaccgtttatggtcccaaaggcccaatggaattccaaattagaacacgagaaatgcaccaaatcgctgaatttggggttgccgctcactgggcttacaaacaagggattaaagcaaaagttgatgttcatgaaatttctgaaaccttgaattggattcatgaacttgtggaattgcgtgaagaagctggtgattcagcagaagattttgtcaaagccgttcaagaagatattttatcagataagatttatgtcttcactccaaatggtgaagtccaagaattaccaagaggttcaggtcctattgactttgcttatgctatccatacaaaagttggtgaccatgcgactggagcgaaagtaaatggacgaatgaaaccattatctgttcaactaaaaacaggtgatcgggttgaaattattaccagctcaagctcttttggaccaagccgtgattggattaatctagttaaaactaataaagccagaaacaaaatcaaacaattctttaagaaccaagataaagaattgtcggtcaataaaggccgtgaaatgttacaagaagctttggaagaaggtggctttgtaccaaatcagtatttagataagaaacatctagatgaagtcttcaataagattagctaccgtaatgcggaagctttgtatgcagcaattggatttggtgagctttcagcaacaacaattgccaatcgtttaactgaaaatgaacgtcgtgaagttgagagagccaaacaaaaagccgaagccgaagagctgatgaagggtgaagttaaacgcgaatcaagtaaaaacattatgaagattcgtcacgatggtggagtcagtgtttctggtattgatagcctacttgttcgtatcgccaaatgttgtaatcctgttccaggggacccaattgtggggtatattacaaaagggcgtggtgtttcagttcatagagctgattgtcaaaatgtgcgaagtatggaagactttgaacaaagacttgttgaagttgaatgggatgattcagaaaacttagtgaaagaatatgtcgctaatattgatgtctatggctttaatcgtcctggattactcaacgatgtgatgcaagttttatcaaattctactaaaaatttgatttcaattaatgctcaacccactaaagataaaaaaatggcgaatattcacatcgctttaggcattaaaaatctatcagatttgactttgattgttgataaaatcaaaatgaccccagatgtttattctgtaaaacggacaaatgcttaa

SEQ ID NO: 10 - protein, 740 aa, Lactococcus lactis
MPKEPDLTGAEVVNICSDYMNETDILLVEKALACASLAHADQYRASGEAYFVHPTQVAGILAKLKLDAVTVSCGFLHDVVEDTNFTQGDLQELFGDEIAEIVDGVTKLGKVEYKSHEEQLAENHRKMLMAMSKDIRVILVKLADRLHNMRTLKHLRPDKQKRISRETMEIYAPLAHRLGIASIKWELEDLSFRYLEEAEFYRIRGLMNEKRTAREALVAEVIGKLQERVEKAGVDAEIYGRPKHIYSIYRKMHDKKKRFDEIYDLIAIRCITETTSDVYTTLGYIHDLWKPMPGRFKDYIANPKANGYQSVHTTVYGPKGPMEFQIRTREMHQIAEFGVAAHWAYKQGIKAKVDVHEISETLNWIHELVELREEAGDSAEDFVKAVQEDILSDKIYVFTPNGEVQELPRGSGPIDFAYAIHTKVGDHATGAKVNGRMKPLSVQLKTGDRVEIITSSSSFGPSRDWINLVKTNKARNKIKQFFKNQDKELSVNKGREMLQEALEEGGFVPNQYLDKKHLDEVFNKISYRNAEALYAAIGFGELSATTIANRLTENERREVERAKQKAEAEELMKGEVKRESSKNIMKIRHDGGVSVSGIDSLLVRIAKCCNPVPGDPIVGYITKGRGVSVHRADCQNVRSMEDFEQRLVEVEWDDSENLVKEYVANIDVYGFNRPGLLNDVMQVLSNSTKNLISINAQPTKDKKMANIHIALGIKNLSDLTLIVDKIKMTPDVYSVKRTNA

SEQ ID NO: 11 - DNA, 73258 bp, Lactococcus lactis
  source 1..73258: mol_type = other DNA; strain = Lactococcus lactis subsp. lactis bv. diacetylactis SD96
  misc_feature 1..73258: note = deleted base pairs 1,823,878-1,897,135
tttatatcgttggtatataaggtttttttgtttatctttttttatttacccctctttttgccccattatttattgttttaattaatctctaagaattaattatattaaatttcatttaattaacaaataaattacatttcaagctctttatttatctttttcttttaaactcaataaacacaagatattgagtgtgtttacataatttaatctatatattgtgtcatcttgtattatctgctaaaataggtgtagaccattgagtaggaaacttcataagttttgaagggagttggtgccttggattattgtttatttaaaaggttattcccttatatgaaagtaaatctcaataatgattgaaaagattatttgcttaattgagcttattcatcttttacttgatatctataaactttttaaatagagtataatagttatcaaaaaaagtcgttaactccccagtaaacgacttcattgctatgttgcaaccataatactatgaaacttaccaatggttttaaagaacttgtacacgcaagttctttttttactttgtctaaatgttgccagtagatactctcttaataataaatatattagcttatttgttagcgtatttgttagcttatttgttagccaatctataaatattttataaactttttgtaaataatttagtaagtatttcagtaacacttttagtaaatgttttagtatattctatggtgtaattattgtataagtaaattatagtatatatagatacttctgtcaatgagaaaaaaaagggggtcttatcaaatggctttatttatcctttatttatcctttataaattagttaaaaatatttaactaatagctaactaaatacctaaccaaatacccaaccaaatacccaaccaaatacctaaccaaatacctaaccaaatacctaaccaaatacctaaccaaatacttaaccaaatatttaaccaaatacttaaccaaatatttaaccaaatatttaaccaaatatttaaccaaatatttaaccaaacttttttataattttctaattgcttcaaaacctgcatctctcttttcagataacaaatgacgataggttttttcaaattgtagtacagtatgacctagaacttttgcgcaaatatccacaggtattcctgaatgaataagataactagctcttgtgtgtcttaatccataaaccgataagttatagggtcttattttcgtatcctccaaaatctttcttaaagctttgttgcttgatactgtacttggtacatcatgtgataagccgtaatgctgaaacactaaattgtatttatttttgatttctaattcggcgttactcaactcctgaactttctttaaaccttttaaaacctttatatcattttcgtttaatggaatatctcggattgaaatattagtttttggagcaacaaatttccaaatttttggcgaggtgttaaatcttcgatatgtatgaattatttcatcttcaaaattaatgtcattccacgtaagagcaatcatttcgccatatctcataccagtagagagtaaaaaataaagtacataagggacaactgattttttataatttaatctttgtcttaattccccttgtagtttcataatatctgatgtactatgaagatatttatcctcaatcagttttgacttcttttgagtatttaaatctactcctagtgtaaagtcatcaacaagcactttatcacttttcgccaactgtatagacttatgaataacactatttattctttgtaaaacatcgtaactaactttttctcccaattcattcattttttgttgatactgacttgtcttaatattttttattggaatatcaaagttttcttgaatcacatttttataataagagtattttctaaaagttgctatttctaaatttttactattaattttcacatcaagccagtaagcatataagttaggcagagtcatttctggatttaacttacttccattttgaatttcttgaaaaataggacttccaactagttgagccattctttttgttttaaaaccaccttgataagctaaactttttcctgattcatctctaatttcatagtgccaaagtttttttactccacgttgcctatatcttattgttgccattatgctacttctccaatccctataatttcacaaataaatttaaatggtgcaaaattttgcttaccattataaaaggtaaagcctgctgaaactgcttttttctttgcatcatgtaccacccttttagcctgagttggtggaatacctaattgttctaaatcttcacttgtaattattggtttgtttattagtttttgattcattaaatttattctcctttatcattttcgtataacgcaaaaaaatagagcaagattcctcttgctcttttacttaacctttaaatatctctgatatatccatgacttgtttttgaataatcaccaattgctaaatctcgaccataagcaccaaagttaaaatatctttgtaacgtttctacgcttagaacttctaaaccgcccatttgctcgattaattcttgtgccaaatcttcatcattttcagcttccacaaagtccaaatcatctttattattgataatatcatcaatactataaatttcaagaaaatcttttaaatgatctctgtcagaaatttcttctaatttttccgcatactcattaagttcctgaattgacgaatactcgcctactttataaccataaaaattttcaaagtcatgtattgcatattcttccccatgctctacatctaaaaataagtctgatttaattcttgaaaaatcaacaggtaattcataccacttaccccttgtttttccattattataactgtttaaatttacaacataaatttttggtgtttccatattcttctcttttctgtttttttattttcctatcacataatgtaattgactagcttgtgcttttgttaatacttggaaagtataaactgtactaaaaccagttcctgactgtgaaatcaagactgaaccgtctgaatgtattccttccacaaaggccacgtgaccgtattgtgggtcagcagtccattgaccgcctactgtttgacctccactaaagctaaccgcactatgaagtgttggagtagtcgttacagtatatcctgcttgatgttgccaatcagcaccatttcccatatacattccaaaagtaataccgaaatcttgggctcgattccatgtaaaccacgtacattgtttatactcataacttgtcgcaagataaccacttgtattaattggtttatctaaagtccaacctgaaggtatatctggagtaattcctgaaccaccaccagttccactatcaccaccagtcgtcatgaacgtgtaccaatattccgcaaaaccttgacgttttggaaggctatcaaccgcccctgcacgttcccactttcgcataatatcttcagtcgtttgcttaacattagttcctttgattaattgtttaagtaacgttgaatctgtaccgtcttgctcaaacatgaactcaacttgaagattagtatctgtccaagattttttctgacttttcgcatagtttagcaagtttactctcctgccactatcccattgagccaaaccgaaagcataaccgcctaaagtggtatccattgcttttgcttcattataggttgcattagattgaataattgaaggttgaagttcactttctgcttgattattaccaagtaaagcagctgccccttctttcgttccaccttcttttagaactctatcccaaatcactttggcagtttgttgctgtggtgtattggtattaatatctgtatctgaagataaatcacttgaacctacaaatagagcaaagaaagctagaattggcaataaaagcaataaaaaaggaaggctaaataatataaccttccgtttaatcacctttttcatttttcacctctctattacttaatcacaaatggattatctggtattttttgattaaatggattgtcattatccttctcccacggctcttttttcttttgctgtgttttttttatcccttcaggcatatttttaccatgtaaaacctttggttttcctgcattttcagcaattattggtgatgcactagaattattcttattgccttcattaatcacatttttattaattgacggtttaatggtgtcagctgattttgatttattttttatcattgggtcaactttatccaatgtttcactttttgattttttatcattggttgaattaggtttatttttaaggaattcctctgctctagcattagttgcatcatacatattttttattccttcacgtttaccattgcgtttattttgaagattattatatgcttcagtatttttacgcaaacgttgtttatcaaacgcttcacggaatttagaacgctttgtattttctgactgattgaatttaccttgacgtttattcattttttctctcaacttttgctcgcgttgattaagtttttgacccttttcagaatcagaaccctttttttgcaatttatctaaggttttattgtagcgttggttatcccaaccatgtctaagcgaaccagtacccgctgtaactttatctttcgcaaatttacctgcaagcatacctgcggttactccaccagttacccctgccaaaacaggagctttcaatgttcccattgctttagattttatatttgacaacatattatccatacgaccagtaaatttcccttgccccatgtgatgagcagaaaatatgccaattatagtcttacgtttaacataaacaaagatataaagtccaatctttactagtgttatgaaaaagtagttactccctaatagagaacttagagctaggctaacaattccgtcaaatagaccaatgagagccgttccaaatacccctaaacctgatagaattaatacaccaaaacttgttttaccccattgaatgattgtcttttcaaaatttggaaaaaatgatgcaatcgctgcaaatggtaatataaacactagaataatgaaaaagaaatccataaagaatgagaaaatagaaaagaataaataaattgccccaagcaacaaagcattaaatatacttgctagtgctgttaaaaatttatcccctagttttttaaaggttaaatgtttatcttttaaatcttcttgtattttcttaatcgaatcgttatcatcatctgaagtaagcaaacggtcaatatcactctgcttaatatttatttttccgtccttatcaatcttgacattatcaaaattaaggtaaaggaaaggaattttaaccattgtggtaaataagtcgtttttatcagttcctgaaacacctgctaccgaaatggttttaacaacacttgttgtaacatcattcattgcatcatgaactttagtcactaggctatttcccccataaacaaaaaacgtacttaaagcaatgattaaaagaacttttctaattccctgcatcatttgcttatgtaaaaaataatatagaagtgtcaaaaacagccccatagcgactatcggaagtaactctggactcataaacttaccgtaaatagtttgagcagtttggaaaacagatgtcctccaatcactaagtattgaattaccatttaatccttgattgacaaaatttacaaactgataaatggtttttgtcacgaaaaattgtgaccgtataaacaccatttgaatacctgcggtaaatgaaggaagtatcacgactttatcacttgcatacatataagaggtatagttatctaaatcttttctagcttcctcatttttggcttcatctactactgtactcggagaattagcatctccatagtcattcatattatcagcaattggtggattgattaaaccactatttcctgtattagaggaaacttttttattagatgtatcatttgagctattctttgaatcatcttttgaattatttttattgtctgaaacagaaaaatcaggggttgacggaggagtaaagcctgaactatctgcataagtatgtagggctaaaacacttaaaaatgcgaatagccccacaaataacaatattttctttttcactatgccaccacctttgtatttttcattttagtttctacagtatcaaataaaactgtgacttctggtaattggaaatcaaccgtaattctagctgtccgtccaaatggatcacgcatgatacattgtcctttagtcatattgtcataccatgcctgcgttgtttcctcaactggaagtttaaagtatttaaggactttatctgtttcagtcggttcacgaaaagcaaaaattgtaccaaaagccgttgaatcatcttctgtttccacatctgaaatagattgtgtaatcaatcctaagaaattgttttgagaacgtccgacacgtttcattgacattaaaatagatgctccgatttctgtcttattaaagaaccacgcttcatcaacaaatattatggtttcttgtgttcggtcacgttccccaaattcacgacaaaattcgcctaaagcatacataataattaatgactctttatggtcgcttgtgattttttcatctgctttaggaagatttaaaccagtaacacccaatactgtaatgcgtgcgtccatatcaacggctgaatttttaccatgagaaaagacaagttccatttgagaatttttaatttttcttaaagccaaattagcacgtgtagaaatcatttcatctgttgaatctaataatctttcaaaaacatttaataaaccaactttttttcctttttctcgttcactcattactgcatctaaagattcaagaaaggcaatttcaaacttatcttcttttgtatatttttcaccaagcaattcttcagtaactttaacagaaattttcttacaagcatcaccatgtaaaaatactagagggtctaatgctccatgattgttttcttcttttgcatctaagctgacaaagtgaatgttattaatataatcaacaactggtttataatacttatattcaatccctcgaattacccagtaaagctgttcttcatcttcaattgcttcacgtgcttgttcaatttcaaactccatttttggaaaatcggcttcatattttgctttgattttcatatattgagccttcatttcatttttagggtcaatataaagcgtcagagctttaagaagtgagtggcaaaggaatagtcctttagttaggaatgatttaccttcaccagtttgtcctacaatggcaaaagctggattgtctgtaatttttcctgcaatcccttgcttattagcttgaaacaggttaagaaatacagcattggtagaactagcaagtgcttttttatagtcacctgcccaagattcgactgcatggtcgattcgcccaataggaaaaccgatatctgaaccaattttttgactagtaaaaaacagattttcacaggctgatgaaagtttcatagcttgaatacaatatttgtctttttcttccaattgttcgataaatcgatttttatagaagaaataaagttggtctgacatgccagaaacaacttgaactccaatatctttcatatcagattgaataatttttttataataattaacctctctcatactttgacctgcaactacaaaagtcgctaggtaattgacaaagagttcacgattatctacaccgtcagtcatttcatcaagaataatacgagcagttgcaagtgaccctttttgatttgaaccattttcataggcttcaatttggttctccttgattcggtctgatgcacgttccccttttgatgctaaagaaaaagcccctttcattttagagaaatggaacttccaatgtaattcaataggaaacggtaatttttgagcatgttctactaaatgaatattatttacaacgtttggcatattagcaataggtaacgcttttactaaactttttccgtagctatttttcaatccaattacaccatttgataatggttcaataactgtatcatcaatattttgtacgctactttcaattgatgctatttcttcatcacgattataattaagacctcgtaaataatctactcgttgataaaagcctgtttctttttttgttgtttttcgaccttgaattaatgaaagatttaactctaaatctccattcgccttgtcataggcttcataccagttcatctcaaattcaagtgtttgacgaacgacagcaactgctttttctttaaagcgcccccacgcatcttttaacatttgtttaatatcacctgaaacttcaaacgattttaaagggatagtcataaagaacttatagttaatcagttgaccaattgaaccatataaatcttcatttgttttttccaagaaataaagcgccatatcataagtatcatctgcgcaatcctccaacagcgccccaaagcgttcttctaaattaatcggaactggtaacattccaatatcaaacgataaattaggtgctaaactagcaaataccgcttcagttctttttttaaattttatctttccttctacgtccatacttgaaataatagacgcaggcacttcatacattgcaatgactgaaccgtcatctcttagtgctaagttatcatgaacagaaataactctattctcaaaatcgactttattcactttttattcttcctccataaattcttctacacttcgcaattttactttttcatatttttgacccttatatatttcaattgctttcaaaccatacccaaattgataaacgataaagtcccacaaaaatactggtggtaatttatcatctgggcgcaaatcagatactttaaccgaaatttgccatgaagcaaaaagcgtaagcgctactttcacttgccattgaaaatatggaatcaattttccaactgtaaaatatagcaaaatgaatactaagaaaaaataagcaaataaggaaagacggactgcctttctcaacgcaaaattatctgtcagttgctgtatccaaaaaggttggtctaatgacaatttataggaaaacttaggcacttcttctttttttgctttttcagacattccgccttcctttctagcctaaaccgagaacttttttcactaaattaccaactggtgtaagcaaaccacctggattattgacgagtagagccaataatgaaccaaaaacaataacaactatcatttttccatactttcgaccgaaaaattcttttcctgcaagataaccaagagctatccaaactacgatttgtccttgttgttgaaaccaagaccaacctccagtcaacccaatatcgccaaagataacattgttaattaatatatttcgtaacattttttctcctattcttttttaccttcaggtaaagtgtgtttaaagtcagtcgcaaaatatgactgcttttgtttttcaatcgtaaattgataattttctacatgtgtaccgagtgcatttttcattgtgatttgaactaccgcatgatatttatctcccccttttgcttcataggtcgcttggtctaaagatttaaattcttgacccttattaagagataacccttttgaaatcaatttcaacgtatcaccgtcagttgtataagcggtaaacaatgattttgtgaacttatccaaatctttttttacaaaatcactcgtttttcctgaatcagaccacgttttagtgggcacttgattttcagttgcttgtaaatcaggaacacttgaaaaataaggttcatctgaaatatagtaagctgaacctactttttggtaaggaatcgtaaacaaagttgtttcatcatgatatttcaccacattttgtttcgttgtatcttttccctttttgacttcttttgttgtgacttcaccagcactataagtcactttataagtcgcttcattatccgtcaaacttatcatgaccgcaccctcaaatttggaaggtgtacgttgaataccttgactaatcactggcagattttgaacaaagtaagcattaatatctttaactgctgtttcttgttctttttcatcttcagggaatgtgaaataagtttgaacaaagccgtcaagataatttttcgcttgcaaatcaagcccattacctgatgctttttgcgtagcactcacttcttcacgaatgattttatgagtgggtttatatccacttgccactagactaactaaagtaacatttgcaataaataaaaaaacaacccctcccacgaccaaagcccctgcctgtttagcagacatcttttttagttttggtggtttttcattcggctgcttctttaacattttgggtttattcttttcaggttttcgacttgaaagaagggaaaatttcgattcctttttttcaggaattttagttgtttctgaaactttcatttcagattcacattcttctttttcaataacttctgaaatatctgattttttatagatattttctagttttaatacttcagcgtcactcaaagattttgatgttgatttagctgaaattccaaattcttgagccttagagagtaactcatttcgctcaacccctaacttgtcagctaattcataaattttaattttgctcatattcctcactttcttgttcaacagctttaaaagatacatcacttcttaaccatttttcagccacttcttcagctttctttttagtatcaaaagtagaagcaaattttaagtgtttagtcgctccaccattggcacaaagatagctaccacgcgtttttacaacgtacattttcacctcttttttcaatttaaaataatatctcaccaaaataatttggcggagttcttgatttatattttgtttttttcattttcgcctaaaataaaaagcagacaattaaatctgctattttctaaacaatatcatgttcataagtttgctcattttcatcacaaaaaataatcacatcttcatcaaattccacaatctcaaagttttctctaataaaatcatggagtttaacttcatttgtttcgcctgtttgtctttcttgtaatttcagaaagaattgacgagcctgtctttcaagtattcgccacgctttttcataagtcaatttattcaccttcttctttagctgattttttcttttcgcaactcttttaatctttcagcaaatacactcataattcaacctccaaagaatttcattaataaataagttagtccaaaaattgctaagaaaatatcaaaataaatagcaattttaaatatttgtttctatccattttctaaccctttctaatttcccaattaggcttttgaatatttgattttggtaaaaatcgaggagcaatcgctattgacagcggagttgaaataggattatatttctcacacagccacatgactttgttttcattaattccagaccaaaattgatttgtaccgtcaggaaaaagaatttgtatatcatacaaatctttatatggtgctttgtgaagtgaaaaatcaatctgtggttgtgctttgctcatatttgttactgtcatatttttatttctcactcatggtgagtatcctttctcttgccttaccatgagtgaggcttttaacattgttatttttcagataaggaaaggcaaaccctacccttatttcaaagatttcgtggactatttatttagcgccactacgtgcttgctcttgtgcttcttccagttttccttttaattcattaaatgatttctttgtgaaattgtatgatgatgaagccttgacaagatttttaatcaagtcatttgtacggtttgcaataacttcgactgtatcatctgaaatagaagtatcattttctacagtacgaagtgctttcccttttcctttgagcttttgagcagcatcttgaaaggcatttcctacattaattacggcttcaatttcatttaaatcagttgcaatttggttaagttctgtttttaaagttggcatatttcatctcttttcttctttattttccctccactaaatttgccttcccttatctgaaaaataactttgtttttatttatgaaaattcataaagttgcttgtgctaaatcattatcaattaaaaagtaaattgccattttatagccttcaggcaaatccgccattcttttttcttgatagagtgaataatattttttacgtgtcattgaatcaggcggaaacatcaagcctatttcattaagcaatcctaatgcttcagtttcatttggaatataaaaaaaaagctggtcataaactccttgctcaaattttttcaaattaaattcatatccactcattttatactggagcattcggacgttgtttggaactctcaacattcttttttgaaaatgttctacattaggtataatcacctctccctttcctcctttaactaattatcaatcaaatatatccgcaagtattgcaataacaaccacaaatacaataatagctacgataactttagttactatttcatcaactggaaaacctgccaatttgaataagctatccatttgcaccccttatcattctaaattccttcaagaatttttcttgtctttctgtaagttctgcttcattaaaaatttcttccaaacgactttcacctgtcagttcttcagctttttttaacaatagataagttgatacaggaccaccacgttcaaaccatgtatattttcggttcaaatcaggcacttcaggacgaacagtaaaatgataggcatccattcgacccatcatatcgtaccattcagtatctaagtttccttccttatcataaaatgtcagattatcattaataattttgaccccttcatcagctaaatcaaaatcttgctcaatatatctctttataaagtcggttgatatttcttttcgcatggaaatttcataacgatttcgcagaccatacatttcacgtatcgcttcaaccgaagtcatttcttgacttgctcgctcataatctttttcgtaaaaccgaaaaaataaatgagtttgtttagatccaaaataaacggttagaccgtcattaatcattccttcttttgtccttcttccaccaagaaccgcagaatagttttgtttacgaccattccaccgaccctcacgggcagaagttaaaagtttgaacaagtcaaaatttccttcttgcgggttatattgttcatcaagtgctaaatcaaaacgtgtgatttttacaaaatcatcattttctggtgcttttttattggcataaagaaaacaatcattaaaaaaatcgtaccaagtacgctgtttttgatgatattcaaactcatattccatttcacggcacgcttgaccgctcatttcaatcagcacaccggcttttatatctttatcatgataataaacattgatgtgaccgtaactaataaaaaaagggtattgataacggttatactcattgagttgaaaattctcaacgtttaaacctaaaatcttagaaacaacttcacgaacatttaaagtctgaaagcgaatatttgtatagtcatttgtaccaacaaaaaccgttgaactctctctttttttgaaaaaggagtgtcttattttttcttccatttcagaagtaacagcacgttcgcctttttccatttttcggtaatagctcaaagatatccctaagaggtcacacataaactgttgagtaacattcgcactagagcgtatatcctttaaatcaatcggtcgttcctcactttctaaattaaaaactacaatatttgtttttaaaataaaaaagatacttaagtttaagcatctttaatcttttgtttcttcctcatctagagctctctgaatactttgttgtcttattttgttgatgtaatttaataccataattacaagtcctcccaaaatagaagagctaataaataaaactactaaaactgcaataaaatatcccataccatctctttgatacatttcagaataagcaatataatttttagtgccttgaataatcgttacaagcattcccacaaaaaacattaccgtaaaagtatttatttttccttgtgttccttccaaattgattcctgcaataatacttctaattatagtgtaaatagctattccaacaacacaaaaaagttctactgcatactgcttaaatggtgcatcaaaaaatatttgttgaacgattaccgatgaaaataggacaacactaattataatataggcttctccctgaatgcgattcatctgagtaataactctcatcacgtattattttattctttttcatcatgttcctcccaaaaaatatcgttcaatgttttttttaaaactctacaaatatctatgcatagccttaaggacggattaaactttccagattcaataagtccaattgtttgtcgagtcacacctacttcatcagctaaatctgcttgactcatttcagcctcaattcgagccattttcattttcttgtttttcaccgttcctccttccttagttatagtataccatatattttgcaaaaagcaatatatatattgctttaatattaacaatttaaaaatgccatgagataaaatactcctaaatagaagaacaattgaatatttatttttataggtactagaaatttacgtatttcattttttcataagagaacacgcttaaactcttgaataacattagtcctgaagtgtcccctttaaaatcaattgagcgttccttactttgtaaaatgtaaaagaggtgacacaagctatactgttggtatgactaggatagttagacttgtgtcacttgtaccccccctcattagcatttgggggggtcaagtccaagctgacggcaagccgtcagccgtgcaatcacacgctcactccttacgctacgctcggagctatgcttacgttcttgcactttttcaaaaaaagcattacgttttaaacgcaataaccttaaaacaaaacccaaaattttctctccaattctttcaagagtaaaccgctgtcgcgcactaccgtttgcgagggcaattcgaccgtggaaaatggcttgcctgcggcaagttgtctaaagacaaacggaaacaagttcctatcatttactcaccgccgattgcatgatgtttaataaaataaaaagcataggatatcctatgcttctcgttggtcaactaattttattatttgagaataatcattcaatgaactcttatatttatcaaagaacgccacttctacattaagaatttctgctaacatttccttagaaatacgcattgaataagttaaactttggacatctgttaaattatttgaaagaacaacatctaacatactcaaaatttttccaggcttatatactggtatttctcggtcaagaggttcaaatattttataattttttttgtgtatttgacgataaaaataactattttgtgccggagtaagatatcctaacttaaatgccttatattctaaagcttgtattgaaacattaaaatattgtttcaacggaatataacgatctggttggctaactttagtacctaccaatttttcaaaagtcttcttaaattcattttcaggtaataataaacttgaagcaaattcattagcttcttgctcttttaaatcatgttcattaatatctaaaagttcaaaatctacagccctatggagtagtaaatgacctaattcatgtgccaaatcaaaatttcttcgagcgtaagatttaccaattgccaagaccaaatacggaacattatcgttggtccaaaggctataagcatcagcgctacttccttcaggcataaaccttgatagcacattaattcctgacatttctattctgtaaagtaaatcatcattatgtatcgaaatgtctaagtgtttacgcgctaataaagcaatttcagaaataagtcttttatcgagtttcttcccttgaagtttttcttcaactacttcaaccaatgaatatatggttttaggagccgatgtcaaataactttctaaatcctcaattagagcatgtgccgcattgacatatacttcttgcatttggattgttttcttcgaagccaaatctccatttctaaaagcaatgcttcctctatttacatgagaaaccaatgggtttgcttcaaaaaatttaatatttactttaaataattttgcaagagttaaaacagtgggtgaaagctttggatttaccttattcgtttcaaattgccaaacagcttgctccgttacatcgagtttttctgcaagctctgccctactcatgccataaagtaatcttaattcttgtagcttttctcctttaaacatttaatcctcttctaagactaggattcgcccttaccttcttgttcacgaacttggaattggtagttattatgctctggcatacgttctttcccaacttccccatcatcaacagtataagcttgacgaatacagtcactaaaatcttgtaattctattgcaccttttttggagtctaaagtatatattccaagataagtaatttgtcctgaaacttgttcaaaatcataacataaagctaaaaaagcctttgttttttctggttttgctagaggaagactattatcttttttctcaagtaattccataaaatcaatagtttcaataggtttctcagtagataacgagttctttctggcataatccactaaatgaaccgctctatcttcctcaaaaccattaataagtgtacttcttctctttacaattaaaacactctgaccatagatagtttccccagtatgttcaaagtacttattaccaaaaccattatccttagtattggtagtgcattcaaaagaaattaaacctttttgaatatcagatttaatagaacaatcaataaagtctcttctagtttttttcaaattactatctagctcaagtgttgcttgagtaaatcgtttagtatttaaaaaatttttatttcctctcactacactattagcatattcctgtagtaactcctgcggtagttcttcatttaccatagttcacctttttcttttagttattttatacaattatatcatataactaaagaaaaaggtacatttttcgtgtttttattaaagtgtctaacatgttttacaatgatttattgtatcatttttttataaaaatataaagtttttttataaattaagcaacatcttctttgtgaaaagtcacaaattctttttcaaactctgcttcatcagctaggacagtatcaatggcttcattcaaagtacattcttctgtttctttagttaagaaaacagtttgtaaagcaataaaatcatcttcattgtaaggtgatttttcacttatagaatatccgccacttgttaaacgctctatggctgttttcacagtttttgaaggttgacccaacttacgagataaagaggtaatcttataagatttttcttccaaaaccccactttctgctcgttcctgttcaacgttagaaattggtggagcttcaagctgaacttcagtagtatcagccaaaactggtaattttgaaagttcctcatgaaaatcataacccttatcaaatggtacagaagggctataaaattcacgtgcaagttcgccattattggcaacataaccacgaccaattttacccttgatgtatttgaaaacttttttctcattttcatctccaaataccatacgataacctgaaccagtcaaacgaccaaccgccaaccgtttcataaagttatcacgtaaagctgatttaagatactcagcgtctggacgttgcatggcaagaattaagaaaacccctgactgacgcccctttaaaacgatttgagttaaaatttgaatcacttcatcaaattctctaaaatctaaactagaaataaaagcagcccattcatcaaaaagaacaaattcaggtgtcaaaccataatcagaatatcgttttcctgccttatagtcagggtgatttgtcatataatcatagcgttcgtccataaattgcatttttgaacgtaaacactcaatcatagactctttatcaaagaacacacgattttcaaaaactggaacatctctcattcccacaaagtctgaacgtttagggtcacatatatccacgtgacctacttttgcaagggctgataaaatagacatcaaaagaactgtttttccgcccccagtacctcctgcaataagcagatgtgggtcagcttcataattccagtaaaccccattcattaactttaaacctagctttttatcaactataacattattaactggaatacgattacgttctccgtcaattgcaaaaacatactcaacaaatgtctttttagaaagaattttattttcattgataaaattttgttcttgataatcagcaaaaaaagttgtttctaaaccgtcagctaaatcaagaaatttttcttgatgaactccaccgtcaattggaaaataagtatgaagttcataagtattaggttgtttcagataaataggcgctatttttaacttttcatgacttcctttttcatctttgactgtttctttttcaactaaattatgcttcacaacatagttcagtaacaatcttttataattattttttgaaaagaaaaggaacttgtgataaagcaaaatcaataaaccaaaagccaccgaaaatgcaatgagcaaaatacctacagcataaattgctagcacttttggtggcaaggtttcaaaatttttataaaagaaccctacaggactagcgacaaataaaccagtaatcaatacaaaaaattcacggtcatacttatctctaagctttaaacgagagcctttacttattgtacgttttgccatttatcatttcttcggttcattgtttttagcttcaggctttggagattgttcaggttttgaattagatttacctactttttcaatcccttcagcattccatttttcacctgaaaagtaattggttctgaactgtccgtttccagtaggtacgagttctttatcagaccaaaaattaattgagcgattcgtcaatttaatttcatcttcatattcaagccctgaaaaatcagcttcagcaggcatttttacaacaatttgcttgtcttgtgcgtcagaataaatattgacctcataataatcaactttatccgttggataacgtttaatttcaccagtttcttcatttggttctccgtcctcaaagaccattactgattcagcatgcaagaacaaaactttacctaatgctttgtcaaaatcaccgatttcttcagtcaacttgttttctttttcaaaaactttacttaattttaacgacatttaatttccccctataattacgctaatttgagcgcttcagcataaacaacatattcttgtgaatatttgccctttggtgtgcgattacgaacataagcaaccgtgacatctgtcaaaacaacttcttgacggcgagtaggtttagcccctttaccatttggcacacgtaattcaaaaccttgaggttgtaacggacttgtagctcgatatttcgtataacctgattcagtagatgtttttgcgactggttcaatggtcttatcgacagtaacttttgaacctaagagtttcgttgcgttaattggaattttaatcatgtttccacctttctttattagtttaatgacatctcggtcaatttatgcgaacgcagggacttgaaccccattcaaccgtgtaacttcgttcgcttaaagaaaaaaaaccaccctcatttaagaaagtgattttttatatataattttattttttaaacttcaaccgtatcaagaccataaccaatactatggtcaagccctaaattaattacttcagcagaatttactgtagaatctacataactttgagcttcagcaaccgtatcacacatttttgtatattttcgtacaccatctacagatacccaacctaaatattttgtagtatgtgttggttgacctgagttccccccccattagaaccattattatttgtattagaaccattcccacctgtgtttgaattagaattagaattattttgtccactattaccttgaccttgattacttgaacctgaaatagtattgttttgagaagtatttgaactaccttgtgaatagttattttgacttcctccaccgtaagaattagaaccactagcaacatcattatttactgtgctatgtgaatcagcacctgtattagaattgttactattattagttgaatttgattcttgtttattagtgtcagcagtttttgcttttgaatcactcgcactcttagattattgtgcctttgtagcgtcatcagcttgtttcttagaagtcgcaagttgtttattcaagctatctttcaaaccttttgaataagagcttgtgagcttagatattgcttgttgagccgttttatatttattttgattcatagcatctttttgatagttactaatggctgtctttgcttcattttctgacttcacacgatttttaataccaactacttgattatcaaacgctttttttacttcagcatttttaactttaagcattgaatcttcagctagtttaatatttttgtcagtaggagttttttcagcaatatcaagtaatttcttactgtctgcttcattagatttttcaagctgttttactttttcggctctagccttacaaagtttaatttttttattttttaaagcttcaatttttctttcttttgcttgatgattagcattagcgactactccagtagtaacaccaccgattaaaacaatcgtaactgctccaccgataatttgctgttttttagtccaattcaaagggtttaaattcttattcataaatacattcctatctttactctaatttaagtttattatatcacaaaccatttggtttgttaaagttaattaacattgtttcttgattcactttttttagtaacatatttattacgctgtgaattcttttctatattcttctaaagcatattttactttagttccttcagggtcaccagtcaactcaatattttctaaaacatctaataaaaattcatctccattcataaggtgactgattccaagcatatacattggagaaactttgaagaaattagcaaactttactatagtttcattactaggtgtccttgctcctctttcgtagcgattataatttgagcgcacaagccctaaagcatcacttacttctttttgtgttaaattgtgactgtgtcttaaatgaatcaaacgttctgaaaaactttccataaaaaaactccattctttttataagggaacaactggaatcgaaccaatatcaccacttataaaagtggttactctagcctattaagctatattcccataaaaaaatctatcctcaaattaagtctgacagatagtatcaacacatctgatagatacttatttcataagatagatttttactaaaaccattatttttctaaaataaaaaactgccatttcggacagtttaaacgactagttttttgtcatttcggacgattttttaaaatttatattttcaataatttgttattgtctttacgcaggcagtaaccttttagaccagaaccctcttcctctcgctttggtcttgatttatatagcgcccttactcaaaatcctcctgctctgcggtatgtatcttccccctcaggcacgcttgttatttcaaagtttgttttaagctttgtgcttgcattgaaaatctccttctgaaatgctataataataattgctacgttatttatataacatttctgtgggagttgccaaagcgcgcttccgcttatttttcttgtagtttttaacgttctacgttaactacatatatgattttaacctattacgttaaaaatgtcaactattaaatgcatttttttcgattttttttggagatgatacaaatatgttctacgaacgcttgaaattgcttgctaaagaaaagaaaaaatctttcaatgaaattgaatctgaactaggatattcaaaaaattctatgtatcactataaaaaagttaaaccaagtagtgataaattatctaaattagccgaatattttggcgtaagctctgattatcttttgggcaatacagatttaagagaacccaaaaaagaaccagttgaccttgaagaattaacttctgatgacggtatcaactgggacgaatggctttcttttggcggtaaaccaatttctgaacatgataaaaataaaatcaaggaaatttttggagatcgcttgaaagattaatttatgaataaagatgaacttattgaactaatcttacttaatatagaaagattaggaattgtcatggcaccgacaaaattagaacagtttgctattataaactgtgaaaaatttattggagtttatgacccttcaagcgtgaccccttttattcttgctcatgaattgatacatgccaagtatggtgataaactgcgccattgcgataatgatattttaagttgtgaagaaaagagagcaaataaagaagcaattattcttttgtgggaaatatacaaagaacaaggtggtaatgcatcttatttttcaaattttattgaaatagctggttgcccttttgaagaatctatcactattctaagagaaatagaaatgaatgatgaaaatattgaagagattactccatgttttgaaggaaatctaagagaatgtgctattcattatatttctggctttgatgtattaaattcaatcaaagtatatgattttcttgaattatataatttatcttatgatttatatgatgaagcagttaaagaatttcagcaacttttagaccaacaaaatttagcctattaaaaaaacttgtaagatttcctacaagttttttatttataataaatatatgaactattttcatttccgattctttgaaccattttcacttccactcgagcaggtttaaaataatcttttgtatctctcataccagatattaagtatctttctaagtccctcgcttcatactcattatcaaaatctctttcttcataatcaaaataatttgttgcatatctaattactcgccactttattttagaatatttttgttcaaattcaatttttttgtttttttgaagttcatctttagttgttgaatctgaattattactccttataggtataatattatctgattttgggtcataattttcaagcccccccattaccattaaaataaaaacagctacaatcactagtaaaactactataaatacccgaattgctagttgattatttagccatgacttgatacccgatagaatagcttaaagtctctggttccagtgatttagctgattttaacaataaagaatacgctaaaagtatcatctctaatttcaattgaaaaccttgaggcgaacgacttttacaacgctcagctcctagatttgtcaaaaaagagaaaactcgctcatcacttttctacgttttgaaaaattagggaacagggtggttgatagagaaaccctgacacttgctaaagcaaatcctcatgaaaatctggggaatcacaagaaaatcaataagttttaaatagaccaatttgactttggtaagaataggtgttactattaagaggtaggttttgtctcgagtttaaatgttttaggaatgcactttttcgcaaaataggatttacagatgatgcgaaaggatgtattatatgaataaaggaacaataaattggtttaacgctgacaaaggctatggttttattatggcagatgatatgcaagatgtgtttgcttatctcttatctattcagggaaatgattttaaaaaatacgatgaaggtcaaaaggttacttttgatatcaaaatgacgtctcgtggtcgttatgcttcaaacgtacataaaagataaatgagttaaagcatggggtgtcctttgcttttttatttattgctattgcaaaaatgaattaaaatgttatactgagtgagggaaagattagttgctttccctacagttcataaaatccatgtcattacgatatggattttattaatggagagtagtttgacattgactataattagtgttattatgaagaagtaaattttgtcatgagtaaaatgttttatagattcacttttcgcaaaatgagatttacaattttgtcgaaaggaaataaatattatggcaaatggaacagtaaaatggtttaacgcagataaaggatttggctttatcacttcagaagaaggcaaagatttgttcgctcacttctcagcaatccaatctgatggattcaaaacacttgatgaaggtcaaaaagttgaatttgatgttgaagaaggtcaacgtggacctcaagcagttaatatcacaaaagcataattgtatctgaaaatcgtgcaaaagctggagtcatgacttcagtttttttatattcagttgcaatgatgagagagtatgatatacttagccagcaagaggaaactcacacttcccatgcataaattagggccactctaatatagagtggtttttttaatgtttattaattaaaattaatatttaaaacatacaaattaataataccatatacccgaattgctagttaattatcaagtcatggctaaataatcaactagcaattcgggtattaatatatctataaattgttgttctagaaactccccaagaatccgcaatcgatttaatttgtgttccactttctatcaatgttttaagaagcttaacgtctttattcctccattgatactattgtaccataacttgaaagcacttatataaatgaacatagaataaattacgggttaatgaacaataaatgcaaatgattttaataatcttaataagaaaagaagagtgttcagaaaatgaccattttctgaacacaatataaaaaaagaaaaacaattattaacgcttttcttttttatgtctaattatttgataatagtccaataatgttgaagcccactttaataaccatactaaatagattaaaattctacgtttgttactctaaaaactagacttaacgtcacttggcatgatgattttctcctaacattaaaatctgtaattacataaactgatgacgacagaaaaatttaactattatcgacccacatctcgcattggccaattacggtagctactgcatcatccatgcctttaggtggatatttgtactttttaagaagtctcttaaccatacgcctcatcccagctcttgcagattctttcttctgccaatccactgttctactcttgcgtaacaagtcagttaattcatgagccaacgaaacgagttcatcattctcataaaaatccttaactgcttctggctttgtaatagcgtcataaaaagctaattcttctgcagttagtcccaagtcgttaccttcttgactggcattagccatatctttcgccatcttcatcagttcttgaatgacttcctcattcgagagcataccgttaagataagctttcatagcacgagaaagcatgtcagagaatttttctgatttcactaggttggtacgtttatacagtgagacttgctcagtaattagtttctttagtaattccactgcaagattcttttccttcattttagaaatttcatccaaaaacttaggatcaaacaaggagaagcccgtgtctatatctgagaataaattgattactccctcactcttgatactagacttcaacagttcgttgatacgctgattgatttccttcaaagataaaggttttctatcacctgtgatgcgtgtcaacaaagtacgaactgcttcaaagaatgcagcctcgaatcgctgttctggtgttaataaagaacgacataatgagagggcttggcgaagcaaaagtgcttgtttgatatattcctctttatccttttctttattaaccgcagaaaggaagttaacacctctactaatcgttttagcacgggtcaaatctgtggcagaatcgtccatgaatttggagtagtcaaagccatggaacatatctcgacaaacttctagtttttcgataaattttggcagagctgtctcagcaatatttgggtctccaaagttccccttatctcgtttcgtataatcattcatggcttgtttcaacgcactagcaattcccacgtagtcaacaactaaaccaccttctttatctttatagacacgatttacacgagcaatagcctgcatcagattatgaccactcataggtttgtaaacatacatagttgccaaactaggaacatcaaatccagttagccacatatctaccacaatagctaatttgaatgggtcatcatcatttttaaatttctttgccatttcgtctttatggctcttgtttccaatgatgtcatgccattcttcggggtcattatttccagaagtcatgaccactccaactttttcagtccagtttgatcgcttttcaagaatcttatggtaaatcttcatcgcaattgggcgagagtaggcaacaatcatagcctttccagttagttcctgcgcacgattgttttcatagtggtcaatgatatcgtcaactaatgcattcagtgtctgctccgcaccaagaattgagtccatgcgtcccagttcttttttgcttctttcaattacatggtactctgccgtttcctccatgaccatatattcattatcaatggcttcaagaacagactcatctagtttcaaatgaatgacacggctctcataataaactgggcgagttgctccatcttctaccgcttgagtcatatcataaacatcaatataattgccaaacacttcagtagttgatttgtccttattagaaattggtgtaccagtaaatccgatataagtagcgtttggtaagctatctcggatttttcttgccgttccgattttcacttggccagttgttgcatcgactttctcctctagcccatactgtccccgatgcgcttcgtcagccatgacaataatatttctacgttcagaaagaggttcatctgactcttcaaatttttgcatggtagtgaaaataattccatttgcttctctaccatcaagcagttcttttaaatttgcacggtcagaagcctggattggtgtctgacgaagaaacttctcacatttcgcaaactgtccaaaaagctggtcatcaaggtcatttctatctgtaagtacaacaaaggttggagaatttaatgcttgttgcaataaatgagtgtaaaagaccatcgacaaggatttaccacttccttgagtatgccaaaacactccgccacggccatctgtttctgtcgcttttttggtcgagttaatcgcttttctcactgcaaaatactgatgataggcagctaaaatctttgcatcgttagagaaacagataaagttttgaaggatgtcaaggaatcgctttttgtcaaacattccttcgatgaaggtgtcaaaagttgcgtactgcgtattttcataactaccgtcaatggttttccattccataaagcggtcttcaccagcagtaattgtgccagcctttgaagtcgccaggtcactcatgacacagaacgcattgtatgtaaaaagacttgggatttccttttgataggttctaagctgtaaaaaggcatccgaagcatccgtttcctcacgagaaggactcttcaattcaaaaaccaccacaggtaaaccattcacaaacacaataacatctggccgtttattgctatattcaataatcgtccactggttaatgacagtaaatgaattattctcgatgttctcaaaatcaacaatcttaactatgcccgcaagatgctcaccatcatgaaaataagacacttcaatcccattttgcagataattcatgaagacttcgttcttttgcagaatcgatccattctcaaaatgttttagcttattcaatgcctcttgaatagccccttctggtaaaaggggattaatccgagttaaagctggctccagctcatcaagatataacggctctgtgtaatcacgttctacatctggaccatattgatatgagtagcctaattcatcttgaaaaagctgaataatcgctttttcgtagttatcttctgcaaaagccatgacttatgcctccttatgcataatattgttaaattcctagttcatcataaactgactgcttataatgataagcacccgtttttccgtctttacgataacttgcatctgacgtgggacaattttctaaagcatattgcattatcttacttccaaatggaataaagatgcttaacttatcatctaaactctttggacaaagggtatctttactcttctttccatttaaatttacaaagataattggtaattctttctttatcgctagttcaatttcccacttaacaaattttgttaaatttttcgtactttctccaattagaagtacaaatactttactgtttgccattcgttctcttagttggcgtttgataggttcttcttgacttgaatctctggcagtatttaaatcgtgggcatcatagaaattaaaatctgttttatcactctgtttctatgccttcatcagatgataatatctaatgtctttatctccatcaaaagcaacatatgttttgtttcggtatgccatattattcattctcctttagttcatataaatttactttttttaacaaatcctcgtgtatgattatctgaagtttagcaggataagtaaactttatcttgctgactttaaatgtccagaccatgatttgaagtaattcattgatgtctatatcctcaaagccatttctaaatctagttatacctgctccaaaaacaggaactgataccgttctctgtgcatatagcctattaatctcattccaaaaagccaaaagaaagttgatatattcctgcatcgttagttctgctttattatcatcatcaaatcggctaaaagcagtaagaataaattcatcatcaattaagacagaactcccaagcttatatcttttctttttcccgacttctcgattcatctcttcaatcaagcctttttcaagacttttatcgctctcaatctgcttatccaatccttgaatatctggaaaatatttttcaatcacctgtccatttaaagacctcttagcaataatcacatcatccacttgtgtgtcaaagtactcattaaaagcaatagctttcaatccatcctgtttaaataaatctcccgattttatttgaactgttgtcgctcctatacttattgaaatatcgtttaaggtattagctctgttccaagtcaaaaaataaatagcaaccaaaaccaagagaacaacaccaccagcaattagtcttaaattcactggaatatcaacaaatattagtacggcagacaaaatggcgaataatgttgcagagcgactgccaaattgcattataacttgcttatcaaaaagtggaacttttttagttttcaaaatgattaacttttttctttcagtatttgataaatcatcatttagcggcgtaataacatacttttatttcgcctgacattaatttcggtaacaaactatctctgagggatattagagattcagtttctttaatgacaaccttcattctttcaaataacggttctactagagagttaaactccttgaaaattttttcactactagggatagctaatttgtaatttggaacagcttttttacttaaatgaagaacggtcgtaccgttaacaaagcccaaacaatattgtttgaaaagttcattctttgtcatcaagagcagtgtgtaattacttattcctgatattttagaattgattttgactaagtccattgaagcaacgattgcatcgtagccgaaatcattaagaagtatttctgaatttccaataatatctgcattttgtgttaagtcagtatgagcaatcaaaatttcatttatagacacaaaatgagtcggctttatcttgtcacttggcactagctctttgaaaccatttgatttaaaaccaccgtttctatcaaaatttttgattgtagccattgcagtagaagattctttaagttctttccctttatatgaataaccattgctgatttcagcaatttctgaaagtgaaacaactttccagcttgagggaatcatgccaatctcagtttcaacaaaatctgttgagttctcgtaatcaatgaaccacgacttgaaaatcgcctgcgccatcaattctaaatgatgatttatcttcctattattctcgatcttgtcatctaatgagccaaggatttttgctatctcatgttgtttctctttattagacggaactgtaacatcgatagcttttacagttttaccagaaacctccttaaaggtagttccactacccatattttcaattttatctttattgaacttgagcaaataatataaaaacaaataatctgtgtcatcattgggaacaatggacttaaaaccctgattagttgaaacctcattttcagctatagccacatacccaataggtgctctagaactcaacaaaatggtatttttaggcattaatctggctgaactacccttcaatccctcttcagtaatatttcgttttccatgtttaatgaatctccctgaaaaggtagataaatcttttggagtaatccaagcaatatctcctccataatactcaggctttttggtagaaggtgttccaccaccaacgacttcacctaaatctcccaaagtacaatttttccattcggacatctaatctctccttatcctataaacttccgatgcgaattcttcacattcgcttgattgaccatcgcataatgcatagtcgtatctatctttacatgcccgagcaggtgttgcacctgctctattggcattcctttatctattgctcgtgttgccaaggttcttctaaacttatgaggatgaactttttgcaaatcaaccctttttccgatttctcgaagtctcgtctccacgccaccaatttgtaatcgctcatgcggttttcctaatgtaacaaacaatgcaagattgctatccgttcgactttccaagtaagtcatcaagtgtattttcgttctggcatcaaaataaaccaaacgttcactaccacctttaccaaataccacacattctcgttcatggaaattgatgtctgcttggtctaatccaaccagctcaccaacacgcattcctgttgaagccaataagtctatcattgccaaatcacgaatttctgtgcaagaatcacgtaataattccaaagattcatccgatagcgtttccttgataggtttatccgtctttatcttatgaattcttcgaacaggacttttcaagatataatcctcatcttctagccaaccaaagaaactactgaaaatgcgacgcatattatcaatcgttaccttgcttgatttgcgttctttttgataatctgacaaatacccacgaagattatcagtatcaatctcacgtattggtatatctgattttgtaagaagcatctgaatagttgattcatagtacttgagcgacttatcagaacacccctcaactctctttgctgacaaaaacattttgagcagccctacgttatctatactttcttgaatagtgttgtccgttctttttacgacttcaacgctttgaaaaacagccgacagtacttgcgttagttctgttaattgcacttgactcaacacccttgacatcctcatttgtatttctgtaataaccttatctttcatctcttcctccaaaaaacagttttcctctattttatcaaggaatgatgagaaacttgtgtactaaatcatcatttagctgagattgccaaaagcattttcgacagctactttattgatttagagccgtatggtgggaatgtaccagttgattggcaatcatcaaacctaacaggtattgcaaactatcttaacggcttggctatgcagaaataccgccctcaaagtaatgaacaaggcttgcctgtcttaaaaattaaagaactccgccaaggttttactgattcaacaagtgacttatgctcttcaagtattcggaaagactatattattcatgatggagatgtgattttctcctggagtggtagtctacttgtcgatttttggactggaggaggctgtggtctcaaccagcacttatttaaagtgacgtctggcacttatgataaatggttctattatgcttggacaaaacatcatcttgaccgatttattaagatggcagctgaccgtgcaacaacgatggggcatatcaaacgtgatgcacttgaaaaagctgaagttctaattccaagcgataaggactatgacgaaatcggtaatatgcttaagccgatttatgaccagataattaataaccgcattgaatccagaaagctagtcgctttgagagactccttgcttccgaaactcatgtcgggagaaatctcggtcaattaagccactaaatgatgatttatcttcttgttttcagcaattctatcatccaaagaacgaagtatagaaccaatttctttttgttcgttaatcggtgggactgggatttctaatcttctaaagtcagttttattaaatttaggttgagcacttccagatactatagatttgagcatgtgttgcccaaatctacttttcatccagtaatagtaaaaatcattatctcctttaggtataaacataatcgagtttggtcctaaagacattggtttagatagactcggagctttgaacaccgtaccaacattagctccaacatttgaaataattatctcattcccaaataatttactttttttcaaaaaatcatatgaatgtcggcttagatatacaaagttcccatcaaattcattgttaaagtcaactaatctaattaagattgcataatccggttcgttcttatattgcacattttctttaagggaagcaaagctgccatttgcaacataatcagtcactaatttggaattttcaagaatcgttccgactccccaatgttcaggaatctttccaaacggagtttctttataattcaaatccaatcgcccccaatctttctttgatttctttctccaattcatgagaacgtgcaaacatttctgaaagctctgaggttagtcgttccatcttttcttcaaatggttcatcgtcttcctcttgttcttcaattccaacatagcgtcctggagtcaagatataatcctgtttctcgatatcttttgtttctacaaccgcacaaaagcctttaacatcttctaactcaccattttgaaaggcttcaaaggtatctgcaattttcttgatgtcatcatcactaaagtcacggtgcttgcggtcaaccatttcacccatcttgcgagcatcaataaagatagtcttgcctttttgtttcttatcacgagaaataaaccaaaggctaacaggaatcgtaacactataaaagaggttagatggtaatgcgactattccttctaccaagtccgcttcaatgatgttttttcgaatctctccctcaccactggtcgtagttgaaagtgacccatttgccaaaaccaatccaatctttcctttcggtgctagatgagaaatcatatgctgcatccatgcatagttagcatttccagtcggtggaacaccgtacttccatctcacatcatcggtaagacgttcgccaccccaatctgaaatattgaatggaggatttgccatgacaaaatcagcctttaatgttgggtgcaaatcattatggaaagtgtctgcttggtgagctccaaagtttgcctcaagtccacgcagagccatgttcatctttgccattttccaagtgtcaggatttgcttcttgaccatagatagacaaatcattgatatttccgctatgttctcttacaaattttgcagattgtacgaacatcccaccagaaccgcatgcgggatcgtacactctgcccttgaagggcttaaggattgcaacgattgtttttacaatactggatggtgtataaaattctccaccattcttcccttccaaagcggcaaatttctcgatgaaatactcataggcacggcctaacaaatcttgctcttcattttcactgtgcaattcaatatttgtaaagatatctactacattgcctagaactcgtttgtcaatatctgggcttgcataaatgataggaagtacatttttcaaagatttattttctttttcgattgcacgcatcgcttcatcaagttttacaccgatttctggcgtgtgagcactacttgaaatcacgctccaacgtgctgtttctgggacaaagaatacatttttctcaatataagcatctctatcttcttcgtctccctcatagtaatcctcagcgtttaacaactcttgatatctgaaatcaaaagcatctgagacatactttaagaaaatcaatccaactacgatttttcgatagtctgaagctgatacatttccccgcatagcatctgctgccgcccataattcattttcaaaaccaatttttgcatttgtctgttttgccattctatattttctcctacttgcttattttttctctggaataatctcaaaagcatctgatactggcaacttctgataaaacgctctctttatattatattccttttgtcgtaatttctctaactctgcttgtacttcttctaaaccgatttgatattcttgtttagccagtttttgctcctcaagagtcaactctgggatagggaagttcttcaagtcctgaaggctgatttgccgaatggttgtccctcgtgaaatcctttctagcagaacttttgccataggactttctagataaatttttaaccattcagggtctgtagttctatttgctctaattctgattaggttggcattgaacagcagcgaatcatcatctttatctaccattccgattttatttgttgtaccacgaatacttaagagtaaatcgcctttctctacccgataatcatcaacattctttacatctgatgtaaccctgactgattcatcataattgattccatcttctgtgatgtctgatattctaatgacttggtactttccgttttcactttcagtggcacgagtattattaaagcctctacttaaacttgctacttcactcagttttagagttttaattctatctagcttattgaaatctgctttgacctcaaggccatcgaaatgataactatcctgaacaacgtatgaatctactacaaggtcaccgttatggatatcatttagattaacagtctccgaaaatcctttaattgattctttgtttccataaacagtagagattttggaaatcaattcatttgacaaaacccgttcacggcgattgacaaccgtcaccatatcttcggtcactttgatgaactgaactcgattttttgcattctccttcttattggtgtttagcactaatattactgttggaatcatcgtgttattaaacataccattaggtaaagaaatgacggtttcaataaaatcaaagttaagaatagattggcggatgattcgatctggcccacctctaaacaatgctccatttggaacaacaatcacagcttttccattatcgggtttaagtgctgataaggcgttgctaacaaaagcccaatcccctgcagaacgtgataaggggccaaatttgaaacggtcatcatttgataaatcagcctctactttcaccccaaatggagggacttgaacgactttgtcaaatttaccatatttgggatactcaggattttgcaacgtatctcctgataagataacagacttttcagcaccattcacaattaaattaatggcagcaaacgtacttaattctgcgttaatttcttgaccgactaagagtgatggctgatgtttgatagcctcaacaagagttccagctgtgcctgctgttgcatccaaaaccatatctccagtctggatattggctaaggaaacaatcagtttattcaaactctccgagttaatatgctcttgatttctcattgcctcaaatccagaatttattaacatcttaggactgtagtcgttaatcagtttcagtatttctcgatacttgccatcagggagtttttcaattaattcttttgttctacttagaaacacttttagctgtgggatattgtcttcttgcataatctcatgttccaatgaatccaagaaggccattttatattgaagcattagagtcttaaaaatgtcgttatttttgtatttcaaatatgcacttgcaactaacatattcggtatatcaacagaactcatagtgccacgccattcatttgttaagtttaattcaattgcttgtgtcatgttttggacctccaccttttactttaacttagtatttgaaatactaagttttgtatcttaaaaaaatatagcccttttctagccgaagaataaactgatattttcttgattaaataaattatatcataatattgatttgatgtcaaataaaaaacatgtatacttaaaattagtattgctaatactaattagtgtttaaccataaaatatcctcactttagttttcgcaattctaaagtcacagaagttcaaatttatgatttgagcttcttttattaaaaaaggagtttgatatgacaaggattcaagatgatttattcgctactgttaatgctgaatggttagaaaatgcagaaattcctgctgataaaccaagaatttcagcttttgatgaattagtactaaaaaatgagaaaaatttggctaaagatttagctgagttatcacaaaacctacctactgataatccagaattgcttgaagcaatcaaattttataataaagcaggagattggcaagcaggagaaaaagcggatttttatgccgtaaaaaatgaacttgctaaagttgaaactttaaatacttttgaagattttaaaaataatttgactggttctgttgcaaagtttgaaaatcaaacgcacccgctctgaactccaaatatcttaggctggtattcccattaataccttgatttcagtagacaccgaaaagccgaagagcgttccatttcttcggttctttttatatattcctcgaagggtctccatgcccttaatcgtggaagaggctgtacggagactttgataaaatttattccgtcgtttaataggtcgatggtcttgttctattaaattgttaagatacttcacagttcggtgctctgtcttagtatataaacccacactctgtaactttctaaaggcggagccaagagaaggtgctttatcggtcacaattgctttcggctcaccaaactgtttatggagtcgttttaagaaagcataggctgcttgcgtatcccgtttctttcgtaaccagatatctaaggttaagccgtccgcatcaattgcacgataaagataatgccaacgtcccttaattttgatataggtttcgtccattttccatgaatagaaggattgtctatttttcttcttccaaagataatagaggactttgctgtactcttgcacccaacgataaatcgtagtatgacaaacatttattccacgatcatataacaattcctgaacttcacgatagcttagattgtaacgcaggtagtaaccaacagcgacaataatgacgtcttttttgaattgtttgcctttaaaatgattcattactctgtcgtctctgtcttttttctcaattttacactaaaatagattttttggaaaactttgcaacagaaccactttgagcaagttggtgcttagctttaagcgtttcactaaaatgcagtacgccccataggtaacaagcgatagctaagcaatctgatgttgcgagatggacgttctttcggttttgaacctcaaggggaacactcgtttgataaagcgtctcaatggttgtcagtaaataaacaaaaacttttggaagtgtgctattataagtcatataagtcgtgcgctttctaatgcttagtggtttaagattaggatagcaagacttatttattttccaatgaaattaactagcaattcgggtataatgtagaatagagctatggagctttaaaattcctgaaatgatttattaagttaatcgagatataatgtgtctaaatgttcgttctgttcgctcctgatgagcatgataatatgtttcggtaattcgggtttttttggaaagtgagaactaaaaatgactaaagtaaataattttgaagaattgattgagaaatctttcgcaagtttattacaaggaaaaacaaaatatatttttttcttgaaaggttttaattattggatcaaaaattttgatctaaatttcttacttgagagtaattctgctggaagcaatgaggagatttcggtcgaaaaaatagttagtaaactttctggaaatgataaatcatactttgcatggtatgaagacattattttattacaagaaaataaattggaaggaattctgaatagcttacaatataagtgtataataatagaaaatgatttctttttaagatattatccttattttaaaggtcaatatgaagatgtttacacaaaagtagaatctttctcagagaacatttatcaaacttactataatgaatttcaaaatattaataatcagtcatatatttcttatgggggtgtgaaagattttaaatgcagatatactaaattttcggacttaatttctcaaaacgaatttgagttagtgctaaatgatgcatccatagaaatttcatcagaattggaaattgttgtttcagattatatacttaaagtacttaatatcctgccacaaagaatatttttatctagagaacttaagaagaataaagttaattttgaattgctgagggaattaggcattaatatagatattatggacgaatcagaagatagtgatcaaatatctagaattaaagaatatgaaaagatactaaagagaaagaacaaaaattatgaattcaaaaatattccgatttatgaaaacccattcgaaaataatattttaaaatctgtaaatcagtctgtcattattaatgaattagttcaaaattctgagaatgcacaagcctcaactacttttaaagatgtttttgtaacagctcccactggtgcaggaaaatctgttatgtttcagataccggcgatttatcttgctgaaaatttgaatttagtgactcttattattactccattaattggtttgatgaatgatcaagtggcaaatattgaatctatgacagattctgcagtaacaattaattcggactatactcccattgagaagcaagagactttagaaaaaataaaaagtggggaaaaatcaattctctatttatcaccagaaactcttctctcaaatagcgatttaacaaatcttattggtgacagaaaaataggattattagttgttgatgaagcccatattgtagcaacttggggaaaaagctttcgtccagattattggtacctaggagattttatcaatcgactaagaaatgggaagaaatcttctcaacattttccaatagctacatttacagccactgctacatttggcggagacgaagacatgtatgaagatatagttggatcattaaaaatgaacccaactcgtttcttaggaaatgtaaaaagagatgatattcactttgatattcggttgaaggaaaaacagctagcttatcgagaagaaaaattagaaaccgttgtaaatacagtaaatgagtcaatcaataataatataaaaacactcacatatactccctatacaaggcaaattcaagaaattattgaaaaagtatctgatagaactaaagtgagtggttatcatgggaaagttaatccatctaaaaaaaatgagagcttaaagaaatttaaatctggtgaacttgaagcaataattgctacaaaagcttttgggatgggtattgatattgatgatattaagcaagtttatcactttgcacctactggaaatattgctgactatgtccaagagataggacgtgcagcgcgtaatccagaaatgaatggtatcgcaagtatggatttctataaagaggatttcagatatattaagcaactttttggtatgtcatcgattaaaaactttcaaattgttggtgttttgcgcaatattaatgaattgtttaagaaaaataagacacgtaattttttagtatctcctgaggatttctcgtatatttttccagaagaaacgattgaaaatatggatacaaaactgaaaaccactgtgctcatgattcagaaagattttgaaatggatccaaggaatggatttgtaccattaatttttaaaccaaggagtatgtttactgaaggatatgtatgcataaatgacgattttttagataatatcaaattgtttggtgcagaaaagtatttttctaaaatttcattgcctcgaaaagtggataagattgataaaagtggaaagatatatacatcagaatttactggagatacttataaattaaattataaattgttgtgggaagaaaagtataaagatatgtcatttggggcttttaaaagggctttccaaatgaacgaactcaaggataattactccgtagggactaagattcttaatagagtaattattgagatcgatggaggagaaaataattttaaattaataaaatcgaaatttaattacgttctggattctttgattacaatctttgacgagttaaaacaggcaaacaaacatttttcagttgatgaattaaagaaaaaaatagtagaaaaaggatttttggatcaaaatcatcaagcagaacctcttgctgcttcaatcatacatctattaaatagagttccagtacagcaattcagtggtaagagattcacagagtttaatagtcaaacaaataagttttcaatcaaaaattcaacatatgaaaaaaggatcaatgaattgaaaaagcattgtagaccaatgttaggaattgatgcgaatacaaaaacaattatctatacttcaactttgaaagatagcatggaaattcttgtgggacaaattctgaaagtattagaattggctgaggtaaaaatctctggaggaaataagcccgagtactttgtccgtgtaaacagtccttatgcgattgaaagaataataaatgacgaaaactaccacagtgagatgttaaaaattgtttatgcaagacatcaaaagtctattgaactcatggaaaagttttttacagagttgaattctgatgaagagcgttgggaatttattgagaaatattttctgggacaaatttaattgtaaaaagaaaaatatgataaactatattaaaatcaagaataaagtatgatattgtttttaaaaattattcaatttttaattcattatcccattatagattcattcaagatgagattattctttggtacactttcaaatattcttaagctttaaaatgcggggaataataggtaattaaatgttcaacctgcattattggtattgggtagatgatagtttgtctggatgtaatttaggaaaagtttcaaaaaggattatatatcaataattatgtaatgaatatctgcgttcaagttattagaaaactattgaggagaaactgacgttaagtcaagtttttagagtaacaaacgtagaattttaatctatttattttaagcacttaccttttcaaattgattaggtgtaaaataccctaaactttgatggattcgttttgaattataaaaggcttcgatgtaccagaaaatactctgataggcttcttcaaagttcttatatttaaattgatacacccactctctttttaaatgtccatgccaagattcaagactggcattatgataagggtatctttaaattcttttgagtagcgttttttcatgtttttgtcctttgtctaaattatacaatagtgactctaagatttaaggataacatcacctctggcgaagaatagaaaattaaaaagccttttaggcttttttataaacgctcgaagagcgtttcttcttatcttgattgtttgcctttaaaatgattcattactctgtcctctctgtcttttttctcaattttacacaaaaatagattttttggaaaactttgcaacagaaccatatttttttataattgagttagactatcaactatttttttagttaattctttttgttgggttgcagttagtttacttgaggttctaattacacatgtaccacaaacttcatgagacccagagttgaataatcctgccccatcgaatgctgataaataagtattgcgtttttgcgcttcttcaggtgatttgtacacttcaatttgtccccctgcatcagtacctttagcaataatatctgatccatctatttgatcttgtacttcagagtccgtaaaataaatagcagaggtgtaccccccaggtttattcaatgatccattgggatcattgctttcggttactgcttcaactcctgtaatggtaggtatcgattttaatttttttattacaaaatctgaagatggattagtcaccaacttatttttattgattgattctttaaaattagataaagccaatttcaattctttttggttatcactatagtcaactgtttttggcaaatcctttattgatttgttaatatcagaagttttagtggggattttgggaactatgaccatatcattctgggattgctttaaaacattgtcaaggttcgccttagttgaattgtccagtggtaattcgttagtttttagacttttttctgcctcttgaataattttatccaaagatttatttttttctttgataggtttataagcactattaaaattatcaaccgcttcctgatgtggttttgcaattccaaagtagtatcctaatcctataccgacaaaaacaactatcatagaaaatatagtaattctttttttcatttgtatctcctcaaatataattatactccatacattagctattatatcactaaaacaaaaaacaacccctattatctaagagttagataataggggttgttttcatttgatttcttaggggttattatataattatagtccctacacaaacatcttttgtaaaaagccttttttctgttctttcaacaaatctaacttacgttgatgaagagagataataaagagctcaaaacttaacagttgtgagctctttttttgtgtgttaaaattaaaggagaaagaacggctcgtaacttagggttattaatagaggtcaaaatatgaaaattgggtatgcacgtgtcagtacttttgaacagaaattagaatctcaaattgaagttctgaaagaagcaggagcagaagaagttttccaagaaaaatttacggggactacagttgaacgtccacaatttaatttagtgctcaaaaagttaacaaatggcgatactttaattgtgacaaagttagatcgattagccaggaacactaaagaagttttagaaattgttcaatcgttatttaatagaggaattaaggtttatatattaaatattggacttatcgataatacaccgacaggtcaacttatttttaccatcttcagtgcttttgcgcagtttgaacgagaccttattgtaaccaggactcaggaaggaaaaaactttgctaaaatccatgatccgaactttaaagaaggacgtcctcagaaatttactgaagaacaaattcaatttgcttatgaattaaagcagcaaggtatgacttacaagatgattgagcgaaaaactggtatcagtattgcgactcaaaaaagaagatttatgaagatcgcaaaaaataaatctattgacaaagactattgaaatgatataattaacataacgaatgattgtgggagtattatctcacagagagtcgggacacgtcgaaaaggtctcggctttttttataaaaaaacactcactccgtcgaaaagtgagtgatgtggaattagtggcgctttttatgtcggtgttttaaccagtccttgaaaagttcaaggacaactcctaccacaatggaagcgacagctgtaacgaatgactgcagaagcatagatctcaccccctccctaaggcatttctgcattggtcaggtgcgccaatttatattgtagcatatcaggaaggattaggctatgtcaaataaaaaatcaaaatattgaaatacaaatattagtaaatttctatattttaaaagtggttaagtttccccgcatcatcactggatttgtaaagcctcacacgttaaatctgttaaggatctcgaaaatagagaaggccgtaagataatcactaactaacaattaataaagaattatgaatcagtagtagtgataaatcaaactgacggaaagaaaaacagttaccccatagctaacgcttgcaatggagaagttagtatttcagaagaaaaaaataatacaagaaaaacctaagaactggaatgtttttaggtttttcattctctactgaaattaatattttgatggggatacaagcataatcaaaatataagtttaagaatttgatactctttttgaaaacgttgcaacagaaacgtcggaggggattttaaagacctttctgtttgatgatttaatcttttataaattatatgatgcatactataggatttaatgataacatcaccattcagagtaccccgacaatccggaaatttatggtaacacttccttaaggaacctataaatattcatcttgggtcaagattttcaattctgggatgatcgttactacaataccattatctagatctgccttatcatcaaacaatatgcccatttttccatctcgttcaatcagtaaacagtttggagtaaatctattttttgactgcgatgacactgaattgagcaatatattttcaatctttttttgaatttcaactctattttgaagaaaataggtataggcattttcttggttctcagttataccttcatcctcaaaatatgatttcgcgtttacgactaaaaaaaatactgtccccaaaaattcaaattctaaatttgtcttccaaccataatcaaactggacatcaccaaaaactttattttccattatttacctccttcactattttcaagagctctcatcatcgaaacacctccactatgtctgatattactgtgaacttcacttggtaatttttgcatcgtttgtccatctctacattcgtgccaagtatagccgttatctttcctccattttgaaacttcttcaggagtatatccagtattccttgcttgagcaatatcagcttttgcaaaattatcaggtcgactgcttgacatttcagaaattttaacatctcctttactaattggtgaaaagtcaggctcaccttcactaaaattaatgccgtcaattccatatttgggtaaaatttctccgaatgtttttccttcagaattcgcaatcttaggaaccgcatctgaatctggaataaatttcgattcgcctcgttccccctcccaatgcccgctcgaaccttcaatgggattgtgagaatatttattttcgaatggcttgtcatattctgtcattggtttatcaaaatccttaccttctacagatttacctaaagggctctcagaaatgttgttttttgaaatttctgacacttccttcatcatcatcggattcatctggcgtcaccccctttcccgtgtatactcaatccattgttggtaaatcttttctctgacttctgtttcatcttttcctttactaaagtcctttatcatacaatgacaaagcgccaatcggacatttctagatgcttccggaataacttcactagataatttcaaaagtccaatagtcaattcctcatcatcccgggcagtcaaaatattgttctctactcgaccacgaatacccagaatactaatcagtctccgggataactgactaaaatcaccatcccatgaattaccgaactccggattttcaatataatctgcaatctgatcaagaatatagagccgatcaactgagctgagtcgcaatatatcaatattattcacgattttagaagcattgaaatctaaatgctccccaactctcagatgcaggagaaattttactacttgattcctaatattttgctcagttagaactttttgagtttccgagtattgaaaagatgtttcgtcaactgtcgctttatgcactttgactaaaacaggattaacccagttattttgataaaccgcggcaacaccattgtcaagctttgcaagttctttaatctgttcatcattcaaattcgcagcttttcccactaattctcgatcactctgggcaggcaatctcaaaataattttggtattcgtattacgaataaccgccatatccagcaactctggagactgatccgcaataataaagccttcgccataagttcgcatttcggcaatcgagttggtcaacatttctacagattttccaagcagattggctccatcagccgattgttccgtcgaggttcgcttgagcaggttatgagcttcctcgagaacagtaatgtgggacaagggactgtttgactttccagaagtcatccgatactcattaagcttcatcaccagcaagcccatgattaaagctttagtttcaactgaccctactcgactcaaatccacaataaccttgctgtcaaatagtgccttatcagataagtcattgacagtaaaaatctgtccattcagcccattggtcaatgaacggactcgcgtcatcaaggcaccgatataatcccctttactgtcagcagaatacctagagttttcaataacctgctcaattttgtccaaaacatctgcaaagttcgggtatttttcaccatttggattctcagatgtttccaggtcccatcctactgacttgtaagcatactccatcgcttcttttaatattgcaggcatcgcagcatacatcggccagcaagcattaaagagttctaccaaacggtcaaggtgctctaggatatgaatatcagcagggaatcggaatggattaatccgtagcaagggcattttcttaggattggtaccatatacagcaacatcccttctatttccaaacgcatttttgtattccccttttgcaggttcaaccactaaaaatggaatattaaaaagtttttctgtcttatcaaggatctcataaatcgtattacttttcccagcccctgtcgaacccgtcacaaatgtgtgcatagtaagcgaatcaagctcaatttcaactctggtcttcccttcaattcccatagaatagatttccccaagcgaaaatgaccgttttgaatcatccgcgttataacacacaacttctttcccaaattctgcgtgatcgacgacagggaagccagttactgattttctaggcaaccccatatgaagagccatttcattaccactaaccagtgatgctggagttacttggagcgtagctaaaggagattcataactaaacactgggtgaatgaaatttgtaatatattccttcagacgtttgtgattttcttggtcatctttcccccaagagttgatagctgaaacttctacgccggttttttcaccccgcattagggaattataggttccagccgccatttctgccgtttgttgactatcagacaaaaagtaggttgcacattcccacattccaagactttcacactcatccattcgtttgagctgtgtgtcgattcgttccaacgtattaatcagcgttttgttctgaatcgtgagttgcaaattatttgacgttccattcgaaacgctgttagtttttgttgctgattcattgacactatcattttccccttcagttcttccctctgtattgccaacacttgaagaatcagaatctcccattgttgtagtgtctttgaagagtgtagctccaattcctgaaactcctgcgactatcatagcacctcctacaggagcacctactccagtagtagccaaagccaccccactaacaactccagcaactgctccaattcctcccagcaacttacttgccatattatccttggatacagattttgaagtactggtacttatattttcagattttgtttttgaaaaagaactacttgtccctttgctagatcctttagaaaacgcatctgaaacactgagtgccatattttgtccatagctcatttgcagatttgcaaagggagagagttgcgtgtagattgtttcataagcgttgcgagtctctgaaagttcttctggatcaacacttttacccaaaacaattcccgtatatttttttccttgcattgccaaagcaaacttttcaagaccttggataaagtggagattatccttggagttttcgtctttgctttttgcaaccactgacacactagtaatgtgctcgcttggaagtctcttcagaagtccttccgccgggtcattcaataagttttcagtctttacgcctggaaattgacctttaagtgaattttccagcatagatttcaaagtgctagttgtccgtttgtcatcaagtgaacgaacacccatatagaattccgtcttttctccatcactatctacgacaataaatacaccgcaattcagattttgcagaacatcaaaaacggcagaaaatttatgagtagaatattcatcggtttgataaaccatttccgtgattttgaaaagttggatgtcattaatttgaatctccgggttttcaagaggtagcacttccagctctgataatcgtgttaaataatgtttgagaaccacgtcatccaccaactgattcgcttctgcaagtttctgtttcaacccgtcaataggtattacattagattgttgcttagttaggtctgaaattttcttggcatcagtcattatttttcaccattagtcgctaccgcagtaacaactcctccaacaacggctccagctgcggctcctaaaataattgatgatcctgatgctgcggcaactgcagcacctactaccgcaataaccacagctccacctgcaacttttacttctctaattggccctcttttcttgtcacgcttcttttgctcgtgataactccaagattctgatgtcttaaacgtagtttttggccgatctgttgctgttggacttttcaaagtcgtttctgggcgtaattttttcacaacctctttaagatgcgaaatcctctcatgggaaaaattacgagtgacttgagccaataatatattcatgtagtcttcgtcccaagacctggaatcttcttttaacactttttcatcgtgcttatcgtaaagtccattcacagttgaagccagacgatccatctcgtcgaattccttaaaacttgggtcaacaagaagactatccttcatcatgatgcggattttactgacattattttcttcaactgcttgtctaaaattattaccaatcttcattttgattgtttttctctttcttgaacttagcggcaatttttttaattgcttcaattttgcttggatttttttccagtcgtatctctaactgcttaagatctttatttttttgttgctgataactttttcttgagctaccttttgtacttacaactttgtccctatatcttttagaatttggaacttccatgggatagatttttttcacgatctgcttgatgtgcctgattctctcccgtgagaaattgcggagcagctgcaccatcagtaaatctaaatattgactgtcccatagactctcgtctgcacgaagttcacgattatcaaaatcgtcatacagtccttcaatttgagaggccagagtgttcattttattgaaagtttttagacttggatcaacaatcaagctgtctttcatcataatccgaaccaagctaatgtttttatttttgacggcttcttcaaaagcttttgtaatcatccgttttcctcagatttccaaaattgactctactttttggttaattgcttctaaccaagcaagtttcttttgcgattctttcatccgttccttagccagatttccactcttagcataatcgtctagctttgttaacttatcaaccaggaccgtatccaaagatttgatttttttcttaaaatcggcgacaactttagttgactgttccttagcatagattgttgcttcagtagcattcgtatttaggttctcttgaacaggggtcaagaattcttgagcaaactcacttccgctaattttttgaacagttttgtattttgttttataataaccttgctcttgcagccaagtccatggtttgtaccaagccttgttgtaattttttacatattcttgtccatcctcctcttcagtatcataaattaaatcttcaacatttactctattatcagttatgctggcttccattaatttcaaaggttcaattttaataccatcagccacatgaatttccgtaaacgcagctaacttgtttcggtattcttcaagaagtgtttcgcttgtgcttcgcaagctgttaatcaccactttatctagttcaacctctaattctggttgaagatttcgtgaaaaggtttcaagttttgctgcttcagcttctgcttgatctatatcaatctcttgacctctaaactgatcaatttgttttgtaatttgcctctggaatttagcaatcacattatcgacagcatcctcagcatctcgacctactttgatcatttggtcatccactttatcaataaacttcttcgcttcattaccatcagaaattttagtttggagttcttttatttgatgactaattctttcactttcttcatgagcggctgccagctcttttttcgcattttcaaaagccccgacttcttcaagattgtggctgaaggtatccacaatgttttttatcttggctgttttcgcatacttttcaacatactgttgaatcgctgcctcgattgaagggattccagaatgaatcaaggcttctttcggattgtttaattttttccctccgctttccttaagttggacaaccgaacttaaattatctgaaatttttcttttaatacttgctggcaaagttgcaaattgatcaaagtaaagttcttcatttcggttcaactttttgaccttagtttcagtttcatctataacatcttcgtctttctcttctccgctttcaagaagtctgatattaagtgccgtcagtgctgaggcaggaaatagatttggattttcaattccgtgattttgaagatatttttttgtatcctcaagaaacgtttctaaggaaccatcatcttttgtccgaccatctaacttattaacgacaaagataaatcggtctttggattgtttccccttaactttcatactttctgatacccggcggagcagcaaattgtcatcatccgttcctcgctcacctgtcatgatataaagcaccaaagcttttgaggattcactcagcatctccacttgacgctttttgtgctgtgggtctcgagaattattcggccctggtgtatctacaatcacaagagcagcatcttctgcgataacaaatggaatatctcctaaaatggtaatttcagacactttttcatcactattgagtctgaccatatcggcataagacaatattggcagttcttcaatgagctcatgctcatcatcatagactttagcctgataagtctcactgtctgtgtctttgattcgcgtgataattgctgtgcaagcttcttgttttgatggcatcagcttggtttgcagcagtgcgttaatcaacgttgatttcccagcactcattgttgcgacgacatcaacttcaaactcattatttttcgcttgttcaaaggcctttttcaacgcaggggctttgagttcttcaaatggcccctcttgaatttctttgaaaacctctgaaatgagtttttccttatcctttgtttcttttgcaggtttgtgatttaatctacgaatcgtcaaatctgacgattcaaaaacttccttcatgtcttcgaaatctaattgtgtcccgaagaattcaacgtcaaaatttaaatcattgcattcttcaacgagaattttaggtaaatcttcaatccactcttgaagacgtatatctgatttcaacaaattgctatccgcttttggaagcttattgtcaattttgatttcagtttcgagcttatagggattgtattttattgcaatattcttcataagatctctctattttctagatatttttctaaagctgtcagtccactattggcataggacgcattaacttcgaccttatcgaagttaaggttataggattccttttttagctttttagtaagtcggtaatattcatccttatcgtcttcatcagataggccttcatactcattttgcttcgctagaaaagcataataagccgatattggaaagacctttggattcttgtaacccagttcgatcaaatcctttatcacctgttcaaggctctcaggtatcgaatcttcattttgattgaatcggtcaaacttattgacgacaaaaacgactttatctttcgggacgttttttgataaccagagcaagtgattttgagcatcctctacacctagatttgtcgcgtctaaaacgcaaatcacctcatcatagtcttcttcaagcaatgctctcttcgtaatcttttggtggtctcgattcgttgctgagtttacaccaggtgaatcaataatgcacaaacgagaattttttttcgaactttttctaaaataggatgaaataattaggcttttatctcgctcagctgctttcaaattcgtaaggcttatatcgaagtccagattatcagtctcaactgcaatcttgtcatcttcaaatggtttattaaacaagtagttaatgtgactagtgcaggcaacttgcgaaatgttcattagaggttttccaatcaaggcattaattagcgttgattttccggcactcatcgttgctgtgatgaatattgttcgcaacggttttttttgaaactcgatgtttttcctgatctgaagtatctgctcttttaggtaagaaaatttttcttcaacttctttattactttctattatttggatcacttcactaagttgatcgaattgttttttattaagcttctcacgccattttgacatagcttgaccaaaatcgcttttttgagttatcaaaatcagatataggtcgcaaagcaaaatgaatctcatttttttcctccagagaacattccaatagcctaatactttctgattttcagttattacagagtctgataggccaagtatcttccaatattgttccaatattccttgtgcgtactttgttcttgtaacatggttcacaagcaaacttaaagactgaaaatatttttcttttgtttgtgatttacagtgcaacatcggatggttatctaaatctaacgctgtgctattctcaaaattcatggttttcctttccctatcatcctcatagtaacccaataacaccatttaattctttctcttttcaaaacggggagatgacggaattaaacactactcaaaagttatttgatactaaaatatctttgtcttgcattatcatatccgtctgcaattattattacatttgattctgcagatgctcctaggaatgaccattccaatgcggaccctactctaaattctttttcctgtacactgcgactatattcgttgaacaatttttcgctgaaaaggaagtgtaacagcaaaaaataaaaaagtataattttaattacccaaactatctaagcaagataacacatcttcaatatcttgatttaaaaattcttccatttcttaaaaatcaaattcttgaaaaacctctgattggtttttgaactttttcattttcatatcaaattatccaaaatctaatcacaatcttattatcttattttatgataataaaatcaaatatcattaggatagatatatcatactaatcagacctttttgagataatccatctgtctaccctcatactctttttcttacattttacgataaaccaggagttataggaaactttgcaagagaaccactttacctaatatattaggttgggtaaacacttctctttcgtcatctaaatcattcaaatctgataatatttcttcaatatcttgatttaataagtcttctatagaatcaaaatcaatgtcttcaaatccatctcgtatttcatttttacaatctatcacatttttttgaaaactagtctttatttttcatcttctatctttgtttcagactattctaacagtttgactgcatctccttgacttctcaaccacatcataagcccttctccaaaagcttctgctcgaatgatccactcccctttggcaacttcctgttcgatttttgctgtgggtattctatctagtaaggcctccaaaactcctgtgtatttgaatcggatatgctgaagctctccggtgttcatgaatagtattcttttgtaaaattccgattcactaaacttctctgaatatggaattgtaaatttttctccatctgataacagattttttactcgatctactctgtaaattgttggtccatcattattagcatctagatggtaagctaataaataaaagtaaaactcagaaaaaacaatactaacgggcttaacgtgatgttttttttctgtgccatcttgacgttgatactcaaaggaaattaagttttgattcctaacagatgtggcaatttcccagattctatttattaaatcttttccatgtttaagttctatatagtggagtctttgatttcctataagctttgaaacttctttggattctttgatattgcattgtctcaataatttatctattaaagcgttaaattcatagtggttgaatgcacgactttcaattaatattttacaaagtgcataaacttccttcttagtgaaccttttgggttctgatagaagataataagcgtttaaagttttatcatagacaattttctcatctgtttcaaaataaaattcacgtaaaaacttaatatcccgatcaaatgttttactaggaatatcaaactctttttttatttcttgctttctaagatactcccccttgtttagtcgttcgttcatccttaaaactcttgaagtcttatcatttttaataaacatttttcctctttaatattatacaactctatcatagacaatttttgggtatataaaaattataccagttttaaaagttaaaagcttaaatggaaagaattccttaaattatgacaccctaaacgtattacccatattttgacccacctatagtgctattgttatagacaggagattaaattatgacacaaaaatcgtattgtaataattgtggttcatcaaatactctagaagactcattttgctcagagtgtggagcgccactaaatcaggggaagccgacaacaggcgaggctaaaactataacaaatagaagagtgaatcattctaaaccgattaaaaaaatcattggaagtatttgtgttggtctgttagtggttgctgctggggtcacagcagttgtaatctatcataatgaacagactcaaagcaaaaaaactaccgcacacgagaaattcctatccagccaggcatctcaagaaaaaaagcaaagttcatcctcattatctgattataaaaagcttcaaaatcttgcaatagattctgttgataaagcctataatactaaaagtagcgctgacatttcctctgctcaagatgtgataagcaaattaaaaaagtcagaccaagcagccttatcacaacgattgaaaaatttaacagatcaaatttctaaagaaaatgaaacgacatcatcttcctcgtctagttctaaaacaaacccacaaacctcttcattattatctggttatagcgatgatcaaattgaatttgcacgtgtaactgaagcaattgttgtttattataaatatacttatcaacctatttcgattagtgtaactaaaaatgaagcaaatcaccaagtgttaccatttgatagttccatagtcattcctcaagaatcagttactttatcgtttagttctgataatacaatggctggaacaactactattacttatacttctaatcacaatggttctattaatttctatcatgaacccaatcattatcaagatgacagatatttaagtgatcctgattgggttaggcaacaatctcaagaaatattaaacgatatgacaaccatagtcattcctacatcagtcgatgataaagcctccttaatcatttccaaaatacaaattaactagaaaaggaataaaacttaacatgaaaaaaaatataatttgcccacagtgtggcgctagtaatacgtcagaaaatatattttgttcagagtgtggaacaccactaaaacaagagagcgctccaaaaggtgagcctaaaactgtaactaatagaagagtgaatcattctaaaccaataaaaaaaatcattggaagtatttgtgttggtcttttagtagttgctgggggagttacagcagtcgtaatctatcataatgaacagattcaaaatcaaaaaactgctgaacaccaaaaattactatctatccaggcatctaaagaaaaagagcaaagttcttcctcattatctgagtataaaaaacttcaaaatgttgcgatatcttccgttgataaagcatataatactaaaagtagtgatgacatttcctctgctcaagatgcgatcagtaaacttaaaaagtcggaccaatcgtccttatctgaacgattgaaaaatttaacagatcaaatttctaaagaaaatgaaacgacatcaaaatcatcttccgaggataatacaaccacgtcttctcaagaacaaataaatcaaccaccaattttaaagaaagaatataaagcctcaggtaatattctacgtttttatccgaaccctgtggggcaagcagatggtgtatacagaattgatacagctaatgatgatggaacagcaaatatggggatgtggagagcttataaacaagtaggaaatattatttatgattctactgatggaaacccaatttctgcgggagatattaatggcggggaacaaaatgcagtatttcaaacagacggtccatcggctcgatataatataacaattgaccctcaatcaggaaacatacaaataggtggtgcaacttttgtaccaactaactaaggagaaaaaatgaaaagttattgtgaaaaatgtggaaatgaacttaaagaaggttccaaattctgtgtcaaatgtgggaatccgaccagtacaaaaaaagaacaaaatatttctcctgattcaatagaaaccgaactaatccaagccgatgaaacaattatggagttttgtacaaattgtggggaagaaatgacccctgaagctgatttctgcatcaaatgtggagtatccaaaaataaaatccataagtattgtatacattgcggttcaactgtaacagcagaacaagatttttgtattaaatgcggaactaaaatttcctcaaactttagtatcgggaacatcaaaatatcagatggaacacaagaacaagtggataaattcgctaaaactacagtcggaagagcctatattaactactggttgaagtggaacaattatacagaaaaagcaactagaccagatttttggtatgcgttgcttgtttgtgccattgtttattttgttctttttatcattactgctattttatcggtaataccattagtcggaaccattgtatctatcatttttgtggtatttattcttataaatcttgccccggttattgccctctatatcagacgctcaaatgatgctggtctcaattggaaatggttcattttagccactgttttatcggcaaacctatttttagctattttaccaagtaaagaataaaaaataaaacaatttagtaaataaggttctgttgcaaagttctaaaattggtaccaagaattttagtttaacaccttaattatgctgatatcctccgtaaaccttaaatttcaagcaataccgaaaaaccgaagagctttccatttcttcggttctttttgtatatgcctcgaatagtctccatgccctttatcgtggttgaggcagtacgtagacttcgataaaatttattgcgtcgtttgattggtcgatggtcttgctctattagattgttaaggtatttcacggttcgatgctctgtcttagtatataattcattactctgtaattttctaaaggcagagccaatagacggagctttatccgtgacaattactctaggttcaacaaactgcttatgtagtcgtttcaagaatgcataagccgcttgagtatcccgtttctttcgtaaccagatatctaaagtcaatccaaactggagctatactataaaagtggacagaaaaatgtgtgttataatctgtctattaagacacaaaaatgaaaggacttttatgtcaggttttaaacgttacgacgaggatttcaaacaatcgctcgttaatctctatcaaactggtaaaacacaaactgaactctgtaaagattatggggtatcttcttctgcacttgcaaaatggattaagcagtactctcaagtgcgtcttgaagataatacggtgttgactgctaaacagattcaagaattacaaaaaaggaatgcccaactagaggaagaaaatctgatcttaaaaaaagcgagtgccatattcatgcaaaactccaagtaagattgaaagccatcttagactccgatttgaacacacgacagtcatgctctgtcgtgttttacatgtcaatcgttccatcaactataactttataaacaagaggccttcgaagcgtgaagtaaaaaatcaacgcttgtgaaaacacctccttgagatttatatgaaagccaagaaaagaattggaacgagagcttttaaaatcattcttctgcgtgattatggcgttaatatttctgaaggctgtatcttaagacttctcaagtctatgacactccctaaaatgtcaaccataaaacctcgttttaaatcaaataaatctcctgtattttcttctgataatctgcttaaacaagaatttaacccgaattccccaaatcaagtttggacaacagatttcacttatatctctataggagctaagcgacatgtttatctctgtgctattcttgacctctactcaaggaaatgtattgcgtgaaaagtaagtgataggattgatgccaaactcgcctgtgataccttagaaatagctataactaagaggaagcctaaggaaccaattatctttcattcggatcaaggaagccaatttaaatcagcttcctttagaaagttattagatgagcatcaattgcttgcttcttactccaagcctggatatccctatgataatgccgtcactgatgtctttttcaagtatcttaaacaacgagaaattaatcgcagaacttatcactccattcaagaagttcaactctcttgctttgaatacatcgaacaattttataacaattataatcctcattccgccaacaatggtttaactccaaattagaaagaagaaaattattttaagaaaatatagctcattttgtgtctaaatatttgacattagtccacctaggattaaattaaaaaaaggaacaccgatattttcaacttttttaaaaccaaatctgataactttattgaaatgaaccaagccaaagcctgtcctgtaaaagacatttgattcattctctgataggctcgccttaagtatttatcctttttttctcctcgttcactttcaaatgataatatgcttaactctattataacaataacattttatcagttttaatgttttttttattgtttttcactctaaaaaaagatataatatcagtaaaactgatatttttgatttaggaggaacaatggaacaattaaaattaaatcaatactttgactattcccttgagccacgtcgtgccatcctctttcaagatgtcaagtctaattatgcttcaattgaatgcgttcagcgaaatttaaatcctctgaccacttctctttgtgtcatgagtcgagcagatcattctaaagggttaacgcttgcctctagtccaacctttaaaaaagtttttggtatgaaaaatgtcagtcgtgccagcgatttaccttttcttatagaatcacgaaagttcaattatccgcaatggtatcgaacacatacagacattcatggacagcgaactgaaccaactttgcaatatgtcgctttcattgaatcttgggcgaaacgcacgtggattgttcctccacaaatgcagctctatgtggactataaaatagaagttacggatattctaaccaattatacatcaattgatgaaattcattcttactcgattgatgagtcgtttcttgacgtaacagaatcactcaatttcttttatcctgagattaaaaatcgttatgaacaaatgaatcgaattgctttagacttgcaacgtgagattcgagataaactcggactatatgtgactgtgggcatgggagacaatcccttgcttgccaaacttgcaatggataactatgccaagcacaatgataatatgagagcgttaatccgttatgaagatgtccctagtaaactatggacgatccctaagatgacggacttctggggaatcggaaaacgaactgagaaacgattaaacaaacttggaatcacttctatcaaagagcttgcgaatgctgacccttttttgctgaagcaaaaattaggaataattggacttcaacattttttccatgctaacggaatcgatgaaagcaacgtaagagaaaaatatactccaaaatctactagcttcagcaattctcaaattctgccacgtgactatcataagcaaagagaaatcgaacttgtgattaaagaaatggctgagaacttagcgataagattgagaaagggcggaaaactagcaagcaatctttcactttatgcaggagctgcttctacttctgaatactcatctattaaggtttctcgcaatatcgaagcaacgcaaaacacaaaggaattgcaagacctcgcaatctctttatttcgtgaaaagtaccaagggggagcgattagacaaatagggataagcgggaatcaactttctgatagttctgttaaacaactgtctttatttgaaagtgttcaagaaaatcaaactaataaaaagcaagaatcactccaaaaagctattgatgagattagagaaacattcgattttctatctatacaaaaagcaagtagtttatccgagggatcacgtgtcatttatcggaacaaactcattggaggtcatgcagcaagtcaagacaaggaggaaaaagatgtcagttgaccgttcatatagtccctacgaggttattaggatgtatcacgatagaggaatgatgaaatggggggcatttgcgactggggaattaactgaagcccaaaatacttttgaaaaagaaaaaaaagaagataagataattcaagcattaccacatcatctcgtgcttcatcttcttaaccagtccttttctaatcaggtgcaaattaaaattcaatatcaagccaaagataaattaactgaagtctatggtaatgtatcagaatttatcaataatcaagtaagagttaaatcaacggataaaatctatctcatatccattaagcaaatcgtaaatatatcataaaaaaagctatccttgttgtatattactacaacaacttggatagccttttatatgacgttaagtcaagtttttagagtaacaaacgtagaattttaatctatttattttaagcacttaccttttcaaattgattaggtgtaagataccctaaactttgatggattcgttttgaattataaaaggctcataagtggagggagaaaatcatgaaatatggatatgctagagtaagtacaatagaccaaaaactagaatctcaaatcgaacaattaaagaatgctggtgcagaagaaattttccaagaaaaatttactggaacaacaaatagtcgcccagcctttataaatttattaaatacattggaatcaggagatactttaatcattacaaagctagatcgcttcgcaagaaatactagagaagcattagcaactattcaagaactttttgacgcaaattgtaaaatgaagttagccaccttgagtcaagcaaaaaacatctgaatctcttatactagaagttcgaccaaatagtataaaaaggaaattcagatgcaagactattctatcacaagccagagcaaagggaaacacctgaccaatcaagaccgtttaaacattgaacgttggcacaataaagaaggactgagcaaccgtgagattgcgcgcttattgaataaagctcatgggacgattgacagagagatgaagcgaggggaaatccagctcaagcggaaggtaaaatactctgctaaaaaggctcaagaaaattatgaaggattgagggctcacagtgttcgtccgagtaaactcacgactgagattgattgttatgtgagtacacgtcttaaagaagacaaggactctttggaagtcattcatcaggctctaaaaggagtttctctgagcagcctctacaattgggtcaactggggctggttagaagccaagcgtcatcatcttttctatccgcagtataaggcggcaaagaaaatcaaacctcgtgcgccaaagcatccttttggcaagtctattgaagaacggcctgagttcattaacaatcgcttagaagtggggcattacgagattgacacggtcattcttacgagagctaagaacaaggtgcttttgacgctcacagagcgaaaataccgcacggagatcattcgcttgattgatgggaaaacagcacaggcggtcaatgatgagctggtggaacttcaaaaaagttatgacttcaaatccattacccctgacaatggccatgaatttgcaaggctctctgaggtcgtaagctgtccagtttactatgcacacgcctacgcgagctttgagcgagggacgaatgaaaaccataaccgaatgattcgtaggcatctgccaaaaggcacaacaaagacgacccctgaagaagtcgccgctatcgagtattggatgaaccattatccaagaaaaatgttcaactataagagttcatatgagatgagcttggctggctaacttggacttgcaatttgcgaagaactttttgataaggatataaaaataaatattttaaatatgggagtgatagataataccgcaactggaaaattaatctttactatttttagtgcctttgctcaatttgaaagagatatgattgttagcagaacacaagagggaaaagaatattcaagaaaaaataatccaaactttaaagagggtagacccaataaatttacagaagaacaaattcaattagcttatgaattaaaacaacaaagaatgacacacaagatgatcgaacgaaaaactggcattagtgtttctactcaaaaaagaagatttaataaaatagtttagttttttatttattagataaataaataattaaataattaaataaacaaattgtttatttttttaattatttgtgttataatagatataggaaggtggtagtacattgaaaatcattacatttacagccattaaaggcggagttggtaaaacaacactaacactaaattatagcgattggttggtaaaaaaaggaaagaaagttttattaattgatttagatcatcaatgcaatttaacaacaatttttcaaccaacacgaagaaataatactattgcagaagcatttaaagatagtgaggaagcacaagaagttatcattgataacattaaagagaatttagacttagtagctggttttattgatttagatgaactaggaagcaaattagaaaacaacagcaataaagaaatgttattatttctttggttaaaaaataattttgaaaaactaaatattggaagctatgactatattttaattgatacacaccctgatttttctacaataacaaaaaatgcagtagcaataagtaattatttagttagtccaattacacctagtgaacatggatattcagcaaaatttgatttagaagctcgtttagaaaaattcagaaaatctttgtttgattataaaactggagaaacttatgttgacgcacaattattttttgtagctaatatgattaaacataacaccagcacatcacatgaattgttaagccacataaaagatgatgaaactgttatttctactatttctgaaagagaaatttttaataaagcaactactaaacacttatctatttttgattttgctaaaacccaagataaaaaagacgataaagttttaaataatattaatgatagtttttcaaaaatatataatcatacaaaataaaggagtttaaattatgagttttgatacatttaacaaagataaagatgaaattgttaaacagaatttaaaagaaacaattgatcctgaaccaaaaaaggaagatgaaattcacagcgtaattagttctattttgaagtctaatgatatgaaaagtgaaaaaagaaaatcagtcactttttcattgactgaaagtcaaataaagaaaattgaagaacaagcacaaaaacacggttttaaaacaaaatctaaactgttagcttatctcatagatcaaatgtaactttaaacaattaattaaataatttgtttatttaataattaaataatttgtttagttgaaggaggttctcatgaaaaaaatcatggacttatggctttacttttatataagttgtatctactttttgccattaattgctctcatgcgttctagcaataaaagtagcaatttcttattaagaagattactatttccctttgaatatttaattcaaagaagacttgaaaaaacaacaaattataatcgtggatcaattcgtgtcgttcatattttcatttggtttttctgtatttttagcttaatgtttgcgacagcaccgctaatttttttccacgaaccacttgaaaatcacaccacattattattatttattacttactactgtatgttagccccattctgtttttggtttcaaccaagaaatctaaaacaataaaaaggagaactttatgtttatagcaactgacaatcaatttacagcaagaggaagattagtagacagtcctcaaattaatatttcagaaaaaacaggaaatgctattgtttcaatcacactagcacaagaacacccttttaaaaaaaatgaagctggggaaagagaatctgtttttattaaatacaccgcaattgatacaaaaaacaaccccattgccagtcgcattgcagaatatgtgacaaaaggatcattagttagtcttattggttatcatgacagttactttaaagaaaactcagagggtaaaaaagaatattttgaagtgaaaagaatttcaacttttagaaatgaagaatcaaaagaaaaaacattggagcgtagaactaaagcataataattaataaaggaaagtgatttttatggaaaaacacaaagtggacttttacaatataccgatcgtagactacttactttcaattggagaaccccttgaaagtgttggtcataactactatcaacataaacatcatgatagtttaaaaattaatcaaaggaaaaactattttgtttggaatagtcgttcatctgaaaaaaattcacgaggtggagttgttcagtatatgcagatcatgcataatttatctttacaagaaacactctcaaaattaagtgaagatttagacggtaaagttcttccagcgatccctaaaaaaagctatcctaaaaaatttaattacccgaattgctagttgattatttagccatgacttgatacccgatagaatatcttaaagtctctggttccagtgatttagctgattttaacagtaaagaatacgctaaaagtatcatctctaatttcaattgaaaaccttgaggcgaacgacttttacaacgctcagctcctagatttgtcaaaaaagagaaaactcgctcaatcacttttctacgttttgaaaaattagggaaaaggattttcttttgcttcatgttcttcctgacaggtgtaattagatcaattccttttaattccagcctatcatgcagtgactgacctaaatatcccatatctccaaggactgttggtgtcccaaattgactcaacacttcctcggtcattgaactatcagccattgaagcaggagtaattgtgtagtctatgacatagcctgattcactgactaaagcatgacatttaaatccatagaagtactgtccctttgtagcattgtagccaacatttgcataatctccaagaactttgcttctgaaattacgaatcgactgacacaaaggaatggggaagctgtcaataatggatacactcattccttcaacctctttaaagacgagtgcttggcgaatgacttggatactcggtaagagggcattacaacggcggacaaagcgagaatattctaggaaattaggaaataaactttgagccaattggtgcttagctttaagcgtttcactaaaatgcagtacgccccatagataacaagcgataactaagcaatctgatgttgcgagatggacgttctttcggttttgaacctcaagggaaacactcgtttgataaagcgtctcaatggttgtcagtaaataaacaaaaacttttggaagtgtgctattataagtcatataagtcgtgcgctttctaatgcttagtgggttaagattaggatagcacgacttatttattttccaatgaaattaactagcaattcgggtaaaaatataatgatgattgaaccccgtgaaaccctcttgatatatactataatataaaaatgaatgtaaaaaaacaaatttttaaaggcacgctctatgcagttgttgccggttgtatgtggggaatttctggcatttttggtcagctttttttccgtgattatcatggtagcccactttggattacatcaacacgtttaacgattgctggtattattttactggtcatgtcttattcgcgagaccataagcgtttttttgatgtctggaaatcaaaaaagaacatgccaacgctctttttgtatgtttttggtggagttttctctgttcagtatttttattatgttgccattcaattgtctaacagtgccacggcgacaattttacaatatacagcacccgtttttattatgctttatttattcattttccaaagacaagtgccaaaacctaaatcggtcttatttgttattttagcaatgataggtgtttttttattgattacaaatggaaacatcaaacatctttcaattagtccacttgcccttgtgacaggccttcttgcgggtgttgcggtcgtaatctattctatcattccacgtcctttattggataagtatggggcgctcaatatgactggatggggaatgatgcttgcgggaattgcttgtaatatcttttatccggtttggcggattaatttccgaattgaacctctttcaattttttacgttttgacgatttctatagtgggaacagcattggcttttttgttgcttttaagagcattccagctaatatcacctcttgttgtttcggttgcaacggccactgaaccactaacctctgtctttttaagtattcccttatttggcttaaaattaacactactagaaatggtggggattgttatggttattgtttcagttattcttttatcaaaatcagaacaagcttaaaagaaaccagtaggtttctttttcttatatacaagattttgacttttttcagaatattctatgatataataaatgcaaagatagcatttaacatcaaaaataaaatgaattgaggaaaatttatgattgagattagagaattaacaaagatgtatggcaagcagtatgctctaaatcatctaagtttaacaattccagaaggtcaaatattcggttttttagggcataatggggcagggaaatccacaacaataaaatgtctgacgagtattattgaaccgacaagtggacaagttttagttgatggtttaaatcttgctgaacatcattcagaaatcaaaaaaagaattggctatgttcctgatactccggatatctttttacaattatctcctaaagaatattggaatttaattgcctcagcttttgcacttgaaacctctgaaaaggaaagacaactagaaactcttacacgacttttcgatattgaagggaaagaatatcaaacaattgaaagtttttctcatggaatgcgtcaaaaagtaattgtcattggggctctcttatcgactcccgatatttggatacttgacgaacccttgcaaggattggaccctcaagcagcttttgatttaaaaaatctgatgaaagcacacgctgcccaaggaaaaacggttattttttctacacatgctttagaagtagcgcaacagctatgtgatcaactagccattttaaaagctggcgaactaatttattcgggaacagttgatgctttattagattcagcagaagatcaatctttggagtcggtttatctcaaaatggtaggtcgtcagcaagaagagctaaatagaggagacgatgatgaatcttcaacaaattaaaagtctttttagtgtcaacctcctttatgccaatcctcaagctacccaacgtcaaagagaaaaaggtaaaaccaaaaatttaaccaaatcacttttgattcaatatgggcttgtctcgctcatcatgttgccatttttcggactgatgatgttagcaattgactttccaaaatatccgggattttttactttttattgtgctctgtttgccctgatgactttttctcaagccttgtcagcaattttaaatattttttatgagagcaaagactttcgagattatctgcctctcccttttgaaaatgttaatatttttgctgctaaatttttagtcgtaatttttgttatcttaccttatttaatgcctcttttggtgctttttggaataacaggttttagggcaaacggagtgattggcctctttctcggattagttggattttttctgtttgctctattattcttactgcttagtacatggttgattacttttttggttcaatctaaaatatttcaacgctataaaaaagtttcccagaccatactattattgatttcaatggttggaatttttgctgggattatgtatctaaataatgctcaaacgacgattagtgcgacaggtaagttgttggatagaggaacttttctgccatttttacctttttggaaatggttagtaaatccgttctctttctatagtttatttagttttctcttttttataatcttattgattgctctttactatgtttttacacttctagtggttccaaaaatgtttaaaattctcttggaagagtcaaaagaaacgagaattgttcaaaaaagaaaaataagaagaacgagaaattttacctcacaattaagaatttataatctaggactaattaaaaatcctaccttatggattcaaatgctgacaggatcctttatttttccgcttgctatgctgcccggtcttttatctagtggtctaaatttgtcaaaattggatttgaaattttttgctatttttcctatcatcggaattagtttagcttttctgaccattaacaatagctctcttccttcaatgattatctcacttgatcgggaaaattttaattattttaaaagcttacccatgaagttgaatttctatctcagagtgaaatttcattttgccttttttattcaactagttcttgacttaatcttatctttagtgattggtattttactgaaagtccctgttaccttgctctttgggctgttaattgggaatgttattggggtatatctgatttcacttcattattttgtcagagattggcatctcttaaatttaaattggactgcgttgactcaactttttacaagaggttctgggggatttcgggtttttatcattttctttgctacgcttttgggagcaggagcactcattggagcaattattgccctgattatctttattccatatcctttagttgttaaccttgccctagtcctccttttaggattttcggtaggagtgatacttcatttttatcagaaaaatttttggaataatctctgataaaaattaacaataaaattactctgctttaggagtaatttttttataaaaaaaattaagaaaacccgaattgctagttaatttcattggaaaataaataagtcgtgctatcctaatcttaacccactaagcattagaaagcgcacgacttatatgacttataatagcacacttccaaaagtttttgtttatttactgacaaccattgagacgctttatcaaacgagtgttccccttgaggttcaaaaccgaaagaacgtccatctcgcaacatcagattgcttagttatcgcttgttacctatggggcgtactgcattttagtgaaacacttaaagctaagcaccaattggctcaaagtttatttcctaatttcctagaatattctcgctttgtccgccgttgtaatgccctcttaccgagtatccaagtcattcgccaagcactcgtctttaaagaggttgaaggaatgagtgtatccattattgacagcttccccattcctttgtgtcagtcgattcgtaatttcagaagcaaagttcttggagattatgcaaatgttggctacaatgctacaaagggacagtacttctatggatttaaatgtcatgctttagtcagtgaatcaggctatgtcatagactacacaattactcctgcttcaatggctgatagttcaatgaccgaggaagtgttgagtcaatttgggacaccaacagtccttggagatatgggatatttaggtcagtcactgcatgataggctggaattaaaaggaattgatctaatgacacctgtcaggaagaacatgaagcaaaagaaaatccttttccctaatttttcaaaacgtagaaaagtgattgagcgagttttctcttttttgacaaatctaggagctgagcgttgtaaaagtcgttcgcctcaaggttttcaattgaaattagagatgatacttttagcgtattctttactgttaaaatcagctaaatcactggaaccagagactttaagatattctatcgggtatcaagtcatggctaaataatcaactagcaattcgggtctataaatataatcatataaaactcctaaatttttgctaatcttttttaattttaaagttatctacatgtattttttcgcaatcaatatcaaatttattctccaatataattttatttatgttttctttatcattttgggaataatagctaaataaatattttgcttcaggggcgcatctgactagttctttaaaataaacccagtctatcttattatatgaatgacctaagattattacttcatgaatatttttatttttaacaaaagactcaattgagtcttttaaaagatttactggtttataaaatcttcctatattttcatcataaaacttattagaattaagctttctagaaccctgttctgcaaacattctttgtgtttccttaaattccttcgtaaaacttgtttctaaccctggtaatttgtcgtctatttctggcgaatgaaatcctaatactacatcttctagattttttacagaaccatgtaaatgtagtacatctttttgttgataaacatcttccatagtatttgtatagttaaaggaaattacagacgaatcttcatctatttcatagatatttttcgtattttttaaaataatcgtttgtatccattcttgaaaactaatatataaatatggaacccatatatatagttcccttaccttattgttaaaatactttaaggcatactcttgaatttcattatgatatatatcagtatcgaatgtactatcttgtttttttattaactttttaaaacaatcaataaatttatgcctctcacctccaatatctaatttgcagctccattcttcttcaaagattataattttcccctctgatatttctgatattattttctcagtctgttcttcaaaatcgctccataaatcatttggttgacaatatctttcaaaatttcgtaaaatatctccttttgatattattaaatcttcatctgtatatacttcatccctatgttccaataaatataatctaaactctgtatatgatgttttcagattgtgtgataaatcaaagccattaccaactatatataatttcattgtatctcccactaacttaactcaataactataactataaatataaatataattatataataatcaaaacagaaaatctcgaaaaaatatgcaaatagaaaaacactaattcaaaaaaataattttttttagttacatatttgaatactcttaacttgaaatgcccccaatttgccccattttatttttcactttaagagcttttctatagtgaacactttgagagctttatagagcctgtatttaactttgaaaactatctaaacaat

SEQ ID NO: 12 - DNA, 82 bp, Lactococcus lactis
  source 1..82: mol_type = other DNA
  misc_feature 1..82: note = deleted tandem repeat ((A)6 to (A)5) upstream of the CodY
tttgttctcctatttttttatattcttgtttttatacaaaatttttcataatcagtataacataaaaaagcatgaaaaacaa